(19) United States
(12) Patent Application Publication
(10) Pub. No.: US 2025/0288690 A1
(43) Pub. Date: Sep. 18, 2025

(54) RNA BASE EDITING COMPOSITIONS, SYSTEMS, METHODS AND USES THEREOF

(71) Applicant: Beam Therapeutics Inc., Cambridge, MA (US)

(72) Inventors: Maria MONTIEL, Cambridge, MA (US); Noah SCHWAEGERLE, Cambridge, MA (US); Justin THOMAS, Cambridge, MA (US)

(21) Appl. No.: 19/093,872

(22) Filed: Mar. 28, 2025

Related U.S. Application Data
(63) Continuation of application No. 18/904,904, filed on Oct. 2, 2024, now abandoned.
Application No. 18/904,904 is a continuation of application No. PCT/US23/65270, filed on Apr. 3, 2023.
(60) Provisional application No. 63/327,140, filed on Apr. 4, 2022.

(51) Int. Cl. A61K 48/00; A61P 9/00; C12N 9/22; C12N 9/78; C12N 15/10; C12N 15/11

(52) U.S. Cl. CPC A61K 48/005; A61P 9/00; C12N 9/22; C12N 9/78; C12N 15/1024; C12N 15/111; C07K 2319/095; C12N 2310/20; C12Y 305/04004; C12Y 305/04005

(57) ABSTRACT

The present invention provides novel RNA base editing compositions, systems, methods and uses. Guide RNAs for site-specific RNA editing of RNA encoding transcriptional coactivators YAP1 or TAZ are provided, and compositions and systems comprising the same with a programmable RNA binding protein (e.g. a Cas protein) and/or a base editor. Methods for RNA editing of YAP1 or TAZ are also provided. RNA editing of YAP1 or TAZ is used for targeting phosphorylation sites, and activating transcription of proteins in regenerative therapy for treating cardiac disease.

### CROSS-REFERENCE TO RELATED APPLICATIONS

This application is a Continuation Application of Ser. No. 18/904,904, filed on Oct. 2, 2024, which is a Continuation Application of International Application No. PCT/US2023/065270, filed on Apr. 3, 2023, which claims priority to U.S. Provisional Patent Application No. 63/327,140, filed on Apr. 4, 2022, the contents of which are incorporated by reference herein in entirety for all purposes.

### INCORPORATION-BY-REFERENCE OF SEQUENCE LISTING

The contents of the ST26 Sequence Listing file named “BEM-014US2_SL.xml”, which was created on Jun. 6, 2025, and is 168,579 bytes in size, is hereby incorporated by reference in its entirety.

### BACKGROUND

RNA base editing may occur through recruitment of adenosine deaminases acting on RNA (ADAR) enzymes to RNA. ADARs recognize adenosine on RNA, however, there is no specific structural or sequence motif that directs ADAR to specific sites on RNA.

Accordingly, there remains a need for highly specific and efficient RNA base editing, with minimal off-target effects. Indeed, precise targeting of ADARs to a single base within the RNA to modify the coding potential of a variety of mRNAs remains a challenge.

### SUMMARY OF THE INVENTION

The present invention, among other things, provides guide RNAs for precisely directing deaminase enzymes to specific sites on an mRNA encoding Yes-associated protein 1 (YAP1) or Transcriptional co-activator with PDZ-binding motif (TAZ or WWTR1), transcriptional coactivators of the Hippo pathway. Directing RNA editing to specific sites could be therapeutically useful in treating a variety of genetic and non-genetic diseases. Some advantages of an RNA editing approach include changes in RNA that are reversible and off-target effects can be minimized by tuning RNA editing activity for example, when DNA editing is lethal.

The present invention, provides in part, a novel regenerative therapy for cardiac disease, based on RNA editing. The present invention is based, in part, on the discovery that a Cas protein with specificity for RNA (e.g. a catalytically inactive or deadCas13(dCas13)), directs an ADAR to a precise base in mRNA, to carry out A-to-I or C-to-U editing, for example, at an RNA base that alters a post-translational modification site in the encoded protein, for example, phosphorylation of Yes-associated protein 1 (YAP1) and/or Transcriptional co-activator with PDZ-binding motif (TAZ or WWTR1) for therapeutic use, for example, in treating cardiac disease. The present inventors have discovered that deamination of YAP1 and/or TAZ RNA using RESCUE or REPAIR editors, targeted by dCas13b at a precise RNA base that prevents the phosphorylation of YAP1 or TAZ proteins, inactivates a kinase signaling pathway, the Hippo pathway that typically leads to degradation of the phosphorylated proteins thereby promoting nuclear localization of YAP1 and TAZ co-activators. Without wishing to be bound to any particular theory, it is contemplated that the YAP1 protein when phosphorylated, for example, at serine 127, or at S127 and one or more YAP1 phosphorylation sites selected from, S109, S164, S381, S383 and S384 or TAZ protein, at S89, or at S89 and one or more TAZ phosphorylation sites selected from S314, S311, S117 and S66 is targeted for degradation in the cytosol. When YAP1 protein is non-phosphorylated (e.g. due to an amino acid change from RNA editing that disrupts the phosphorylation site), YAP1 enters the nucleus and interacts with the TEAD family of transcription factors to activate transcription of pathways involved in replication, organ size, stem cell renewal and cell survival such as, but not limited to Wnt, c-Myc, cyclin D, FGF1, CTGF, TGFβ/SMAD (FIG. 1). This leads to transcriptional activation by YAP1 and TAZ of pathways involved in replication, organ size, stem cell renewal and cell survival. The present inventors have been able to transiently fine-tune the function of YAP1 and TAZ proteins by recoding amino acids using RNA editing to induce activation of cell proliferation pathways in damaged myocardial tissue.

RNA editing of YAP1 and TAZ overcomes numerous challenges posed by other therapeutic approaches. Homology-directed repair of double-stranded breaks has low in vivo efficiency. DNA base editing or prime editing are relatively efficient at editing genes without double-stranded breaks, but they use large constructs that are not currently deliverable, for example, by AAV vectors. Further, DNA editing strategies pose a risk of permanent off-target mutations in the genome. Since messenger RNA (mRNA) molecules exist transiently within the cell and encode genetic information for the production of proteins, a strategy to edit YAP1 and TAZ RNA rather than DNA, therefore, allows for the editing to occur at a transcript level, without the risk of creating permanent off-target mutations in the genome. Furthermore, the transient nature of RNA means that RNA editing is potentially reversible and controlled over time i.e. titratable. The A-to-I and C-to-U editing of YAP1 and TAZ mRNA prevents degradation of YAP1 and TAZ protein in the cytosol, and results in YAP1 translocation to the nucleus, where YAP1 and TAZ interacts with TEAD and transactivates genes involved in cell proliferation and regeneration, thus RNA editing of YAP1 and TAZ provides a novel regenerative therapy for cardiac disease, among others. RNA editing may be advantageous compared to genome editing because it allows a finer degree of control, e.g. inducible RNA editors or systems with a built in “on” switch for continual dosing.

In some aspects, provided herein is a guide RNA, comprising (a) a scaffold for binding a nucleic acid programmable RNA binding protein; and (b) a spacer sequence having one or more regions complementary to a target mRNA encoding a Yes-associated protein 1 (YAP1) or a Transcriptional co-activator with PDZ-binding motif (TAZ or WWTR1); wherein the spacer sequence comprises a single or double nucleotide mismatch to an adenosine or cytosine in the target mRNA.

In some aspects, provided herein is a guide RNA, comprising a scaffold for binding Cas protein, a spacer sequence having one or more regions complementary to target RNA, a mismatch corresponding to adenosine or cytidine in a target RNA, wherein the guide RNA is directed specifically to a target site in an mRNA encoding Yes-associated protein 1 (YAP1) or Transcriptional co-activator with PDZ-binding motif (TAZ or WWTR1).

In some aspects, provided herein is a guide RNA, comprising a scaffold for binding Cas protein, a spacer sequence having one or more regions complementary to target RNA, a mismatch corresponding to adenosine or cytidine in a target RNA, and chemically modified bases, wherein the guide RNA is directed specifically to a target site in an mRNA encoding Yes-associated protein 1 (YAP1) or Transcriptional co-activator with PDZ-binding motif (TAZ or WWTR1).

In some embodiments, the guide RNA comprises a scaffold for binding a nucleic acid programmable RNA binding protein, wherein the nucleic acid programmable RNA binding protein is a Cas protein, Type VI Cas protein, Cas13 protein, or Cas13b protein.

In some embodiments, the guide RNA comprises a spacer, wherein the spacer comprises between 4-15 consecutive nucleotides that are perfectly complementary to a target mRNA.

In some embodiments, the guide RNA comprises a spacer, wherein the spacer comprises a sequence having at least 70%, 75%, 80%, 85%, 90%, 95%, 98%, 99% or greater identity up to 100% identity to a sequence selected from the group consisting of SEQ ID NO: 7-SEQ ID NO: 33. In some embodiments, the spacer sequence comprises a sequence selected from the group consisting of SEQ ID NO: 7-SEQ ID NO: 33.

In some embodiments, the guide RNA comprises a sequence having at least 70%, 75%, 80%, 85%, 90%, 95%, 98%, 99% or greater identity up to 100% identity to a sequence selected from the group consisting of SEQ ID NO: 34-60.

In some embodiments, the guide RNA comprises any one of SEQ ID NO: 34-60.

In some embodiments, the guide RNA is bound to a base editor.

In some embodiments, the guide RNA is bound to an mRNA encoding YAP1 or TAZ.

In some embodiments, the guide RNA comprises a region that binds to an ADAR protein.

In some embodiments, the guide RNA comprises a scaffold capable of binding RESCUE or REPAIR base editor and directs it to a target site.

In some embodiments, the guide RNA recruits endogenous ADAR to a target RNA site for base editing.

In some embodiments, the guide RNA directs a Cas protein and a RESCUE or REPAIR base editor to a target site. In some embodiments, the guide RNA directs a Cas protein and a RESCUE base editor to a target site. In some embodiments, the guide RNA directs a Cas protein and a REPAIR base editor to a target site. In some embodiments, the guide RNA directs a Cas protein and a deaminase enzyme to a target site.

In some embodiments, the guide RNA comprises a spacer of between about 30-50 nucleotides. In some embodiments, the guide RNA comprises a spacer of between about 30-36 nucleotides, 36-40 nucleotides, or 40-50 nucleotides.

In some embodiments, the guide RNA comprises a spacer of between about 30-36 nucleotides. In some embodiments, the guide RNA comprises a spacer of about 30 nucleotides. In some embodiments, the guide RNA comprises a spacer of about 31 nucleotides. In some embodiments, the guide RNA comprises a spacer of about 32 nucleotides. In some embodiments, the guide RNA comprises a spacer of about 33 nucleotides. In some embodiments, the guide RNA comprises a spacer of about 34 nucleotides. In some embodiments, the guide RNA comprises a spacer of about 35 nucleotides. In some embodiments, the guide RNA comprises a spacer of about 36 nucleotides.

In some embodiments, the guide RNA comprises a mismatch about 17, 24, 25, or 26 nucleotides in length from the scaffold. In some embodiments, the guide RNA comprises a mismatch about 17 nucleotides in length from the scaffold. In some embodiments, the guide RNA comprises a mismatch about 18 nucleotides in length from the scaffold. In some embodiments, the guide RNA comprises a mismatch about 19 nucleotides in length from the scaffold. In some embodiments, the guide RNA comprises a mismatch about 20 nucleotides in length from the scaffold. In some embodiments, the guide RNA comprises a mismatch about 21 nucleotides in length from the scaffold. In some embodiments, the guide RNA comprises a mismatch about 22 nucleotides in length from the scaffold. In some embodiments, the guide RNA comprises a mismatch about 23 nucleotides in length from the scaffold. In some embodiments, the guide RNA comprises a mismatch about 24 nucleotides in length from the scaffold. In some embodiments, the guide RNA comprises a mismatch about 25 nucleotides in length from the scaffold. In some embodiments, the guide RNA comprises a mismatch about 26 nucleotides in length from the scaffold.

In some embodiments, the guide RNA comprises chemical modifications at 5′ and/or 3′ end. In some embodiments, the guide RNA comprises 3× 2′O-methyl and/or phosphorothioate at 5′ and/or 3′ end.

In some embodiments, the guide RNA comprises a 6 nucleotide extension sequence.

In some embodiments, the extension sequence comprises UUmC*mG*mA*U (SEQ ID NO: 4). The notations mC*, mG* and mA* refer to chemically modified bases, for example, in some embodiments, mC* refers to 2′O-methyl cytosine, mG* refers to 2′O-methyl guanine and mA* refers to 2′O-methyl adenine.

In some embodiments, the scaffold sequence is derived from Prevotella sp. or Riemerella antaipestifer. In some embodiments, the scaffold sequence is derived from Prevotella sp. In some embodiments, the scaffold sequence is derived from Prevotella sp. or Riemerella antaipestifer.

In some embodiments, the scaffold sequence has at least 70%, 75%, 80%, 85%, 90%, 95%, 96%, 97%, 98%, 99% or greater identity up to 100% identity to any one of SEQ ID NO: 5 or 6.

In some embodiments, the scaffold sequence has at least 70% identity to SEQ ID NO: 5. In some embodiments, the scaffold sequence has at least 75% identity to SEQ ID NO: 5. In some embodiments, the scaffold sequence has at least 80% identity to SEQ ID NO: 5. In some embodiments, the scaffold sequence has at least 85% identity to SEQ ID NO: 5. In some embodiments, the scaffold sequence has at least 90% identity to SEQ ID NO: 5. In some embodiments, the scaffold sequence has at least 95% identity to SEQ ID NO: 5.

In some embodiments, the scaffold sequence has at least 96% identity to SEQ ID NO: 5. In some embodiments, the scaffold sequence has at least 97% identity to SEQ ID NO: 5. In some embodiments, the scaffold sequence has at least 98% identity to SEQ ID NO: 5. In some embodiments, the scaffold sequence has at least 99% identity to SEQ ID NO: 5. In some embodiments, the scaffold sequence has at least 100% identity to SEQ ID NO: 5.

In some embodiments, the scaffold sequence has at least 70% identity to SEQ ID NO: 6. In some embodiments, the scaffold sequence has at least 75% identity to SEQ ID NO: 6. In some embodiments, the scaffold sequence has at least 80% identity to SEQ ID NO: 6. In some embodiments, the scaffold sequence has at least 85% identity to SEQ ID NO: 6. In some embodiments, the scaffold sequence has at least 90% identity to SEQ ID NO: 6. In some embodiments, the scaffold sequence has at least 95% identity to SEQ ID NO: 6.

In some embodiments, the scaffold sequence has at least 96% identity to SEQ ID NO: 6. In some embodiments, the scaffold sequence has at least 97% identity to SEQ ID NO: 6. In some embodiments, the scaffold sequence has at least 98% identity to SEQ ID NO: 6. In some embodiments, the scaffold sequence has at least 99% identity to SEQ ID NO: 6. In some embodiments, the scaffold sequence has at least 100% identity to SEQ ID NO: 6.

In some embodiments, the scaffold sequence is a Prevotella sp. scaffold of SEQ ID NO: 5.

In some embodiments, the scaffold sequence is a Riemerella anatipestifer scaffold of SEQ ID NO: 6.

In some aspects, provided herein is an engineered, non-naturally occurring composition for modifying a target RNA base, comprising: (a) a Cas protein, and (b) a guide RNA molecule directed to an mRNA encoding Yes-associated protein 1 (YAP1) or Transcriptional co-activator with PDZ-binding motif (TAZ or WWTR1).

In some aspects, provided herein is an engineered, non-naturally occurring composition for modifying a target RNA base, comprising: (a) a Cas protein, (b) a base editor, and (c) a guide RNA molecule directed to an mRNA encoding Yes-associated protein 1 (YAP1) or Transcriptional co-activator with PDZ-binding motif (TAZ or WWTR1).

In some embodiments, the composition comprises a Cas protein, wherein the Cas protein is a Type VI Cas protein.

In some embodiments, the composition comprises a Cas protein, wherein the Cas protein is Prevotella sp. PspCas13b or Riemerella anatipestifer RanCas13b.

In some embodiments, the composition comprises a Cas protein, wherein the Cas protein is a catalytically inactive Cas13 or dead Cas13 (dCas13).

In some embodiments, the composition comprises a base editor that comprises one or more RNA binding domains fused to a nucleotide deaminase.

In some embodiments, the composition comprises a base editor, wherein the base editor is ADAR or active fragment thereof. In some embodiments, the base editor is ADAR2 or active fragment thereof.

In some embodiments, the catalytically inactive Cas13 or dead Cas13 (dCas13) is a Type VI Cas protein.

In some embodiments, the base editor comprises a deaminase domain fused by a linker to catalytically inactive or dCas13, and a nuclear export signal. In some embodiments, the deaminase is exogenous. In some embodiments, the deaminase is endogenous.

In some embodiments, the composition comprises a base editor, wherein the deaminase is fused to an N-terminus of dCas13. In some embodiments, the composition comprises a base editor, wherein the deaminase is fused to a C-terminus of dCas13.

In some embodiments, the composition comprises a base editor, wherein the base editor is an adenine deaminase.

In some embodiments, the composition comprises a base editor, wherein the base editor is a REPAIR editor.

In some embodiments, the composition comprises a base editor, wherein the base editor deaminates adenosine to inosine (A to I).

In some embodiments, the composition comprises a base editor, wherein the base editor is a cytosine deaminase. In some embodiments, the composition comprises a base editor, wherein the base editor is a RESCUE editor.

In some embodiments, the composition comprises a base editor, wherein the base editor deaminates cytidine to uridine (C to U).

In some embodiments, the composition comprises a base editor, wherein the base editor has at least about 80% identity to a nucleic acid sequence of SEQ ID NO: 1-3.

In some embodiments, the composition comprises a base editor, wherein the base editor has at least about 85%, 90%, 95%, 99% or greater identity up to 100% identity to a nucleic acid sequence of SEQ ID NO: 1-3.

In some embodiments, the composition comprises a guide RNA, wherein the guide RNA comprises a sequence having complementarity to a target RNA sequence that comprises an adenosine or cytidine.

In some embodiments, the composition comprises a guide RNA, wherein the guide RNA comprises a scaffold for binding Cas protein, a spacer sequence having one or more regions complementary to target RNA, a mismatch corresponding to adenosine or cytidine in the target RNA; and chemically modified bases.

In some embodiments, the composition comprises a guide RNA, wherein the guide RNA comprises a spacer of between about 30-36 nucleotides.

In some embodiments, the composition comprises a guide RNA, wherein the guide RNA comprises a spacer of about 30 nucleotides.

In some embodiments, the composition comprises a guide RNA, wherein the guide RNA comprises a mismatch about 17, 24, 25 or 26 nucleotides in length from the scaffold. In some embodiments, the composition comprises a guide RNA, wherein the guide RNA comprises a mismatch about 17 nucleotides in length from the scaffold. In some embodiments, the composition comprises a guide RNA, wherein the guide RNA comprises a mismatch about 24 nucleotides in length from the scaffold. In some embodiments, the composition comprises a guide RNA, wherein the guide RNA comprises a mismatch about 25 nucleotides in length from the scaffold. In some embodiments, the composition comprises a guide RNA, wherein the guide RNA comprises a mismatch about 26 nucleotides in length from the scaffold. In some embodiments, the guide RNA comprises a C or U mismatch. In some embodiments, the guide RNA comprises a C mismatch. In some embodiments, the guide RNA comprises a U mismatch.

In some embodiments, the composition comprises a guide RNA, wherein the guide RNA comprises chemical modifications at 5′ and/or 3′ end.

In some embodiments, the composition comprises a guide RNA, wherein the guide RNA comprises 3× 2′O-methyl and/or phosphorothioate at 5′ and/or 3′ end.

In some embodiments, the composition comprises a guide RNA, wherein the guide RNA comprises a 6 nucleotide extension sequence.

In some embodiments, the composition comprises a guide RNA, wherein the extension sequence comprises SEQ ID NO: 4.

In some embodiments, the composition comprises a guide RNA, wherein the scaffold sequence is derived from Prevotella sp or Riemerella anatipestifer. In some embodiments, the guide RNA comprises a scaffold having 75%, 80%, 85%, 90%, 95%, 96%, 97%, 98%, 99% or greater identity up to 100% identity to SEQ ID NO: 5 or SEQ ID NO: 6.

In some embodiments, the composition comprises a guide RNA, wherein the scaffold sequence comprises a Prevotella sp scaffold of SEQ ID NO: 5.

In some embodiments, the composition comprises a guide RNA, wherein the scaffold sequence comprises a Riemerella anatipestifer scaffold of SEQ ID NO: 6.

In some embodiments, the modification of targeted RNA base changes one or more post-translational modification sites of the protein encoded by the target RNA.

In some embodiments, the modification of targeted RNA base changes one or more phosphorylation sites of the protein encoded by the target RNA.

In some embodiments, the modification of targeted RNA base changes a single phosphorylation site in the protein encoded by the target RNA.

In some embodiments, the modification of targeted RNA base changes the encoded amino acid from a serine, threonine, or tyrosine to an amino acid that cannot be phosphorylated.

In some embodiments, the target RNA encodes a protein that is a transcriptional activator, co-activator or signaling protein.

In some embodiments, the transcriptional activator, co-activator or signaling protein is a protein in a kinase signaling pathway.

In some embodiments, the transcriptional activator, co-activator or signaling protein is a protein in a hippo signaling pathway.

In some embodiments, the target RNA encodes Yes-associated protein 1 (YAP1).

In some embodiments, the target RNA base modification modifies YAP1 phosphorylation sites at serine 127 (S127, corresponding to mouse S112) and/or serine 109 (S109, corresponding to mouse S94).

In some embodiments, the target RNA base modification modifies YAP1 phosphorylation at S127. In some embodiments, the target RNA base modification modifies YAP1 phosphorylation at S109. In some embodiments, the target RNA base modification modifies YAP1 phosphorylation at S127 and/or one or more of S109, S164, S381, S383 and S384.

In some embodiments, the composition comprises a target RNA that encodes Transcriptional co-activator with PDZ-binding motif (TAZ) or WWTR1.

In some embodiments, the composition comprises a guide RNA that targets TAZ mRNA of SEQ ID NO: 9-21. In some embodiments, the target RNA base modification modifies TAZ phosphorylation sites at serine 89 (S89). In some embodiments, the target RNA base modification modifies TAZ phosphorylation sites at S89 and/or one or more of S314, S311, S117 and S66.

In some embodiments, target RNA base modification modifies S127 on YAP1 and S89 on TAZ. In some embodiments, target RNA base modification modifies one or more YAP1 phosphorylation sites selected from S127, S109, S164, S381, S383 and S384 and one or more TAZ phosphorylation sites, selected from S89, S314, S311, S117 and S66.

In some embodiments, the guide RNA of the method comprises a sequence having at least 70%, 75%, 80%, 85%, 90%, 95%, 98%, 99% or greater identity up to 100% identity to a sequence selected from the group consisting of SEQ ID NO: 34-60.

In some embodiments, the guide RNA of the method comprises a sequence selected from the group consisting of SEQ ID NO: 34-60.

In some embodiments, the guide RNA comprises a spacer sequence having at least 70%, 75%, 80%, 85%, 90%, 95%, 98%, 99% or greater identity up to 100% identity to a sequence selected from the group consisting of SEQ ID NO: 7-33.

In some embodiments, the guide RNA comprises a spacer sequence selected from the group consisting of SEQ ID NO: 7-33.

In some embodiments, the target RNA is modified in organs selected from a group consisting of heart, liver, lung, kidney, brain, CNS or skin.

In some embodiments, the target RNA is modified in the heart. In some embodiments, YAP1 RNA is modified in the heart. In some embodiments, TAZ or WWTR1 RNA is modified in the heart.

In some embodiments, the Cas protein is a catalytically inactive or dead Cas protein and the base editor is a RESCUE or REPAIR base editor, wherein the modifying of a targeted RNA base is from adenosine to inosine or cytidine to uracil, and wherein the RNA base modification results in a modified phosphorylation site of YAP1 or TAZ protein. In some embodiments, provided herein is an engineered, non-naturally occurring system for RNA editing, comprising the compositions described herein.

In some aspects, provided herein is a method of modifying a target RNA base, the method comprising administering a composition comprising: (a) a Cas protein, (b) a base editor, and (c) a guide RNA molecule directed to an mRNA encoding YAP1 or TAZ, wherein the administering modifies the target RNA base.

In some embodiments, the method comprises administering a composition comprising: a catalytically inactive or dead Cas protein, a RESCUE or REPAIR base editor, and a guide RNA molecule; wherein the modifying of target RNA base is from adenosine to inosine or cytidine to uracil; and wherein the RNA base modification results in a modified phosphorylation site of YAP1 or TAZ protein.

In some embodiments, the catalytically inactive or dead Cas protein in the method is a Type VI Cas protein.

In some embodiments, the catalytically inactive or dead Cas protein in the method is dCas13.

In some embodiments, the Cas protein is PspCas13b and the base editor has at least about 80% identity to SEQ ID NO: 1 or 3.

In some embodiments, the Cas protein is RanCas13b and the base editor has at least about 80% identity to SEQ ID NO: 2.

In some embodiments, the Cas protein has at least about 85%, 90%, 95%, 99% or greater identity up to 100% identity to a nucleic acid sequence of SEQ ID NO: 1-3.

In some embodiments, the guide RNA of the method comprises a sequence having at least 70%, 75%, 80%, 85%, 90%, 95%, 98%, 99% or greater identity up to 100% identity to a sequence selected from the group consisting of SEQ ID NO: 34-60.

In some embodiments, the guide RNA of the method comprises a sequence selected from the group consisting of SEQ ID NO: 34-60.

In some embodiments, the guide RNA comprises a spacer sequence having at least 70%, 75%, 80%, 85%, 90%, 95%, 98%, 99% or greater identity up to 100% identity to a sequence selected from the group consisting of SEQ ID NO: 7-33.

In some embodiments, the guide RNA comprises a spacer sequence selected from the group consisting of SEQ ID NO: 7-33.

In some embodiments, the modification of a targeted RNA base, changes the post-translational modification of the encoded protein. In some embodiments, the modification of a targeted RNA base, changes the serine 127 (S127) of YAP1 protein to an amino acid that cannot be phosphorylated. In some embodiments, the modification of a targeted RNA base, changes the serine 109 (S109) of YAP1 protein to an amino acid that cannot be phosphorylated, thereby activating YAP1. In some embodiments, the modification of a targeted RNA base, changes S127 and/or one or more YAP1 phosphorylation sites selected from S109, S164, S381, S383 and S384 to an amino acid that cannot be phosphorylated, thereby activating YAP1. In some embodiments, the modification of a targeted RNA base, changes S89 of TAZ protein to an amino acid that cannot be phosphorylated, thereby activating TAZ. In some embodiments, the modification of a targeted RNA base, changes S89 and/or one or more TAZ phosphorylation sites selected from S314, S311, S117 and S66 to an amino acid that cannot be phosphorylated, thereby activating TAZ. In some embodiments, the modification of a targeted RNA base changes one or more YAP1 phosphorylation sites selected from $127, S109, S164, S381, S383 and S384 and/or one or more TAZ phosphorylation sites, selected from S89, S314, S311, S117 and S66 to an amino acid that cannot be phosphorylated, thereby activating YAP1 and/or TAZ.

In some embodiments, the method leads to an increase or decrease in expression of a target RNA.

In some embodiments, provided herein is a method of treating disease by administering to a subject in need thereof, an effective amount of the composition disclosed herein, wherein the composition activates or inactivates a signaling pathway by a post-translational modification.

In some embodiments, the post-translational modification is phosphorylation. In some embodiments, the disease is caused by activation of a kinase pathway. In some embodiments, the disease is a degenerative disease.

In some embodiments, the disease affects one or more organs from heart, lung, liver, kidney, brain, CNS or skin. In some embodiments, the disease is a cardiac disease. In some embodiments, the disease is caused by phosphorylation of YAP1 protein. In some embodiments, the disease is caused by phosphorylation of TAZ protein. In some embodiments, the disease is caused by phosphorylation at YAP1 S127. In some embodiments, the disease is caused by phosphorylation at one or more YAP1 phosphorylation sites selected from S127 and/or S109, S164, S381, S383 and S384 to an amino acid that cannot be phosphorylated, thereby activating YAP1. In some embodiments, the disease is caused by phosphorylation of TAZ protein S89. In some embodiments, the disease is caused by phosphorylation at one or more TAZ phosphorylation sites selected from S89 and/or S314, S311, S117 and S66 to an amino acid that cannot be phosphorylated, thereby activating TAZ. In some embodiments, the disease is caused by phosphorylation at one or more of YAP1 or TAZ phosphorylation sites. In some embodiments, the disease is caused by phosphorylation at one or more YAP1 phosphorylation sites selected from S127, S109, S164, S381, S383 and S384 and/or one or more TAZ phosphorylation sites, selected from S89, S314, S311, S117 and S66 to an amino acid that cannot be phosphorylated, thereby activating YAP1 and/or TAZ.

In some embodiments, the administering of the composition deaminates adenosine to inosine or cytosine to uracil, thereby mutating a phosphorylation site and preventing phosphorylation of YAP1 or TAZ.

In some embodiments, the administering of the composition in vivo is at a molar ratio of base editor:guide RNA of between about 1:1 to 1:50. In some embodiments, the administering of the composition in vivo is at a molar ratio of base editor:guide RNA of about 1:1, 1:5, 1:10, 1:15, 1:20, 1:25, 1:30, 1:35, 1:40, 1:45, or 1:50.

In some embodiments, the administering of the composition increases replication, organ size growth, stem cell renewal and cell survival.

In some embodiments, the administering of the composition to cardiac tissue or cardiomyocytes results in decreased scarring or fibrosis.

### Definitions

In order for the present invention to be more readily understood, certain terms are first defined below. Additional definitions for the following terms and other terms are set forth throughout the specification.

A or An: The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Base Editor: By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). In various embodiments, the agent is a biomolecular complex comprising a protein domain having base editing activity, i.e., a domain capable of modifying a base (e.g., A, T, C, G, or U) within a nucleic acid molecule. In some embodiments, the polynucleotide programmable nucleic acid binding domain is fused or linked to a deaminase domain. In one embodiment, the agent is a fusion protein comprising one or more domains having base editing activity. In another embodiment, the protein domains having base editing activity are linked to the guide RNA (e.g., via an RNA binding motif on the guide RNA and an RNA binding domain fused to the deaminase). In some embodiments, the domains having base editing activity are capable of deaminating a base within a nucleic acid molecule. In some embodiments, the base editor is capable of deaminating one or more bases within a nucleic acid molecule. In some embodiments, the base editor is capable of deaminating a cytosine (C) or an adenosine (A) within RNA. In some embodiments, the base editor is capable of deaminating a cytosine (C) and an adenosine (A) within RNA. In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas13 (dCas13) fused to an adenosine deaminase. In some embodiments, the base editor is fused to an inhibitor of base excision repair, for example, a UGI domain, or a dISN domain. In some embodiments, the fusion protein comprises a Cas nickase fused to a deaminase and an inhibitor of base excision repair, such as a UGI or dISN domain. In other embodiments the base editor is an abasic base editor. Details of base editors are described in International PCT Application Nos. PCT/2017/045381 (WO2018/027078) and PCT/US2016/058344 (WO2017/070632), each of which is incorporated herein by reference for its entirety.

Base Editing Activity: By “base editing activity” is meant acting to chemically alter a base within a polynucleotide. In one embodiment, a first base is converted to a second base. In one embodiment, the base editing activity is cytidine deaminase activity, e.g., converting target C⋅G to T⋅A. In another embodiment, the base editing activity is adenosine or adenine deaminase activity, e.g., converting A⋅T to G⋅C. In another embodiment, the base editing activity is cytidine or cytosine deaminase activity, e.g., converting target C⋅G to T⋅A and adenosine or adenine deaminase activity, e.g., converting A⋅T to G⋅C.

Base Editor System: The term “base editor system” refers to a system for editing a nucleobase of a target nucleotide sequence. In various embodiments, the base editor (BE) system comprises (1) a polynucleotide programmable nucleotide binding domain (e.g., Cas13), a deaminase domain and a cytidine deaminase domain for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domains selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Catalytically Inactive: As used herein, the phrase “catalytically inactive” refers to a substantially reduced cleavage activity but may show detectable activity to a certain degree. In some embodiments, the cleavage activity is diminished by at least 50%, 55%, 60%, 65%, 70%, 75%, 80%, 85%, 90%, 91%, 92%, 93%, 94%, 95%, 96%, 97%, 98%, 99% or greater identity up to 100% identity. Exemplary functional diminishment are described in Zhang, et al., Two HEPN Domains Dictate CRIPR RNA Maturation and Target Cleavage in Cas13d, Nat. Commun., 10:2544, 2019; Slaymaker, et al., High-Resolution Structure of Cas13b and Biochemical Characterization of RNA Targeting and Cleavage et al., Cell Rep. 26:3741-3751, 2019.

Cleavage: As used herein, cleavage refers to a break in a target nucleic acid created by a nuclease of a CRISPR system. In some embodiments, the cleavage event is a single-stranded RNA break. In some embodiments, the cleavage event is a double-stranded RNA break. In some embodiments, the cleavage event is a break in an RNA: DNA hybrid. In some embodiments, the cleavage event is in messenger RNA. In some embodiments, the cleavage event is in circular RNA.

Complementary: As used herein, complementary refers to a nucleic acid strand that forms Watson-Crick base pairing, such that A base pairs with T, and C base pairs with G, or non-traditional base pairing with bases on a second nucleic acid strand. In other words, it refers to nucleic acids that hybridize with each other under appropriate conditions.

Clustered Interspaced Short Palindromic Repeat (CRISPR)-associated (Cas) system: As used herein, CRISPR-Cas system refers to nucleic acids and/or proteins involved in the expression of, or directing the activity of, CRISPR-effectors, including sequences encoding CRISPR effectors, RNA guides, and other sequences and transcripts from a CRISPR locus. In some embodiments, the CRISPR system is an engineered, non-naturally occurring CRISPR system. In some embodiments, the components of a CRISPR system may include a nucleic acid(s) (e.g., a vector) encoding one or more components of the system, a component(s) in protein form, or a combination thereof.

CRISPR Array: The term “CRISPR array”, as used herein, refers to the nucleic acid segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The terms “CRISPR repeat” or “CRISPR direct repeat,” or “direct repeat,” as used herein, refer to multiple short direct repeating sequences, which show very little or no sequence variation within a CRISPR array.

CRISPR-associated protein (Cas): The term “CRISPR-associated protein,” “CRISPR effector,” “effector,” or “CRISPR enzyme” as used herein refers to a protein that carries out an enzymatic activity or that binds to a target site on a nucleic acid specified by a RNA guide. In different embodiments, a CRISPR effector has endonuclease activity, nickase activity, exonuclease activity, transposase activity, and/or excision activity.

crRNA: The term “CRISPR RNA” or “crRNA,” as used herein, refers to a RNA molecule including a guide sequence used by a CRISPR effector to target a specific nucleic acid sequence. Typically, crRNAs contain a sequence that mediates target recognition and a sequence that forms a duplex with a tracrRNA. In some embodiments, the crRNA: tracrRNA duplex binds to a CRISPR effector.

Ex Vivo: As used herein, the term “ex vivo” refers to events that occur in cells or tissues, grown outside rather than within a multi-cellular organism.

Functional equivalent or analog: As used herein, the term “functional equivalent” or “functional analog” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Half-Life: As used herein, the term “half-life” is the time required for a quantity such as protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

Hybridization: As used herein, the term “hybridization” refers to a reaction in which two or more nucleic acids bind with each other via hydrogen bonding by Watson-Crick pairing, Hoogstein binding or other sequence-specific binding between the bases of the two nucleic acids. A sequence capable of hybridizing with another sequence is termed the “complement” of the sequence and is said to be “complementary” or show “complementarity”.

Indel: As used herein, the term “indel” refers to insertion or deletion of bases in a nucleic acid sequence. It commonly results in mutations and is a common form of genetic variation.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Mutation: As used herein, the term “mutation” has the ordinary meaning in the art, and includes, for example, point mutations, substitutions, insertions, deletions, inversions, and deletions.

Oligonucleotide: As used herein, the term “oligonucleotide” generally refers to polynucleotides of between about 5 and about 100 nucleotides of single- or double-stranded nucleic acid. Oligonucleotides are also known as “oligomers” or “oligos” and may be isolated from genes, or chemically synthesized.

PAM: The term “PAM” or “Protospacer Adjacent Motif” refers to a short nucleic acid sequence (usually 2-6 base pairs in length) that follows the nucleic acid region targeted for cleavage by the CRISPR system, such as CRISPR-Cas. The PAM is required for a Cas nuclease to cut and is generally found 3-4 nucleotides downstream from the cut site.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably.

Prevent: As used herein, the term “prevent” or “prevention”, when used in connection with the occurrence of a disease, disorder, and/or condition, refers to reducing the risk of developing the disease, disorder and/or condition.

Phosphodegron: A phosphodegron is one or a series of phosphorylated residues on the substrate that directly interact with a protein-protein interaction domain in an E3 Ub-ligase, thereby linking the substrate to the conjugation machinery as referenced in Ang, et al., SCF-Mediated Protein Degradation and Cell Cycle Control, Nat., 24:2860-2870, 2005. In some embodiments is repetition of amino acids in the mRNA that has a pattern, HXRXXS (X is any amino acid, H is histidine, R is arginine, and S is serine).

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

REPAIR base editor: RNA editing for programmable A-to-I replacement (REPAIR) is a fusion of inactivated Cas13 (dCas13) with the adenine deaminase domain of ADAR2, which efficiently performs adenosine-to-inosine (A-to-I) RNA editing.

RESCUE base editor: RNA editing for specific C-to-U exchange (RESCUE) is a base editor which performs both cytidine-to-uridine (C-to-U) and A-to-I RNA editing, is a fusion of inactivated Cas13 (dCas13) with evolved ADAR2, a cytidine deaminase.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

RNA guide: The term RNA guide refers to an RNA molecule that facilitates the targeting of a protein described herein to a target nucleic acid. Exemplary “RNA guides” or “guide RNAs” include, but are not limited to, crRNAs or crRNAs in combination with cognate tracrRNAs. The latter may be independent RNAs or fused as a single RNA using a linker (sgRNAs). In some embodiments, the RNA guide is engineered to include a chemical or biochemical modification, in some embodiments, an RNA guide may include one or more nucleotides.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

sgRNA: The term “sgRNA” or “single guide RNA” refers to a single guide RNA containing (i) a guide sequence (crRNA sequence) and (ii) a Cas nuclease-recruiting sequence (tracrRNA).

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215 (3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Target Nucleic Acid: The term “target nucleic acid” as used herein refers to nucleotides of any length (oligonucleotides or polynucleotides) to which the CRISPR-Cas system binds, either deoxyribonucleotides, ribonucleotides, or analogs thereof. Target nucleic acids may have three-dimensional structure, may including coding or non-coding regions, may include exons, introns, mRNA, tRNA, rRNA, siRNA, shRNA, miRNA, ribozymes, circular RNA, plasmids, vectors, exogenous sequences, endogenous sequences. A target nucleic acid can comprise modified nucleotides, include methylated nucleotides, or nucleotide analogs. A target nucleic acid may be interspersed with non-nucleic acid components. A target nucleic acid is not limited to, single-, double-, or multi-stranded DNA or RNA, messenger RNA, circular RNA, DNA-RNA hybrids, or a polymer comprising purine and pyrimidine bases or other natural, chemically or biochemically modified, non-natural, or derivatized nucleotide bases.

Therapeutically effective amount: As used herein, the term “therapeutically effective amount” refers to an amount of a therapeutic molecule (e.g., an engineered antibody described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

tracrRNA: The term “tracrRNA” or “trans-activating crRNA” as used herein refers to an RNA including a sequence that forms a structure required for a CRISPR-associated protein to bind to a specified target nucleic acid.

Treatment: As used herein, the term “treatment” (also “treat” or “treating”) refers to any administration of a therapeutic molecule (e.g., a CRISPR-Cas therapeutic protein or system described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF THE DRAWING

Drawings are for illustration purposes only; not for limitation.

FIG. 1 is a schematic of the Hippo signaling pathway. Phosphorylation of YAP1 or TAZ proteins in the cytosol leads to degradation of the YAP1 and TAZ proteins. The non-phosphorylated YAP1 and TAZ enters the nucleus and activates transcription of genes involved in replication, increasing organ size, stem cell renewal and cell survival.

FIG. 2A is a schematic that shows the domain structure of base editors. ADAR2 comprises two double-stranded RNA binding domains, dsRBD I and dsRBD II, and a deaminase domain. A REPAIR base editor comprises a dPspCas13b under the control of a T7 promoter fused to a deaminase domain via a linker, and a nuclear export signal. A RESCUE base editor comprises a dRanCas13b under the control of a T7 promoter fused to a cytosine deaminase domain via a linker, and a nuclear export signal. The linker comprises a sequence GSGGGGS (SEQ ID NO: 68). FIG. 2B shows a deamination reaction of adenine to inosine catalyzed by ADAR. FIG. 2C shows a deamination reaction of cytosine to uracil catalyzed by ADAR. FIG. 2D is a schematic that shows guide RNA with a Cas13b scaffold, and a spacer that recognizes mRNA comprising an A-C mismatch. FIG. 2E depicts the structure of a chemically modified base, 2′-O-methyl-3′ Phosphorothioate. FIG. 2F is a schematic of guide RNA recruiting Cas13b fused to ADAR to a specific site on mRNA.

FIG. 3 is a graph that shows results of cytosine to uracil (C-to-U) conversion percentage achieved with a base editor comprising a RESCUE editor targeting YAP1 phosphorylation site at human serine 127, which corresponds to mouse serine 112. RNA editing was evaluated as percentage C-to-U conversion in a variety of cell types, and results are shown in Hela cells at 6 hours, 12 hours, and 24 hours, AC16 cardiomyocytes at 12 hours and 24 hours, primary human hepatocytes at 12 hours, Hepa 1-6 cells at 12 hours and 24 hours and primary mouse cardiomyocytes at 24 hours post-transfection.

FIG. 4 is a graph that shows results of adenine to inosine (A-to-I) conversion percentage achieved with a base editor comprising a REPAIR editing targeting YAP1 phosphorylation site at human serine 109, which corresponds to mouse serine 97. RNA editing was evaluated as percentage A-to-I conversion in a variety of cell types, and results are shown in Hela cells at 6 hours, 12 hours, and 24 hours, AC16 cardiomyocytes at 12 hours and 24 hours, primary human hepatocytes at 12 hours, Hepa 1-6 cells at 12 hours and 24 hours and primary mouse cardiomyocytes at 24 hours post-transfection. Guides contain a spacer region of 30 nucleotides in length with a mismatch 17 (30ntMM17-U) or 26 nucleotides in length (30ntMM26-U) from the scaffold.

FIG. 5 shows exemplary immunofluorescence images of endogenous YAP1 localization in HeLa cells. Antibodies specific for the various phosphorylation states of YAP1 were used, including total YAP, non-phospho (active YAP) and phospho-S127 (inactive YAP) antibodies. Cells were plated at low, medium and high confluencies to visualize the localization of YAP within the cytoplasm or nucleus. YAP1 localization in the nucleus was predominantly observed in low density wells, whereas YAP1 was predominantly observed in the cytoplasm when plated at higher densities.

FIG. 6 depicts a diagram of a heart and the site of injection. Base editor and guide RNA are injected into the apex of the tissue.

FIG. 7 is a graph that shows results of cytidine to uracil (C-to-U) conversion percentage achieved in cardiac tissue in vivo by injecting mice with a RESCUE base editor and a guide RNA targeting YAP1 phosphorylation at human serine 127, which corresponds to mouse serine 112, at 12 hours or 24 hours post-injection at a ratio of 1:1, 1:10 or 1:35 of base editor:guide RNA. The results showed about 0.5% base editing using a 1:35 base editor: guide RNA, 24 hours post-injection.

### DETAILED DESCRIPTION

The present invention, provides in part, a novel regenerative therapy for cardiac disease, based on RNA editing.

In some aspects, provided herein is a guide RNA, comprising: (a) a scaffold for binding a nucleic acid programmable RNA binding protein; and (b) a spacer sequence having one or more regions complementary to a target mRNA encoding a Yes-associated protein 1 (YAP1) or a Transcriptional co-activator with PDZ-binding motif (TAZ or WWTR1); wherein the spacer sequence comprises a single nucleotide mismatch to an adenosine or cytosine in the target mRNA.

In some aspects, provided herein is a guide RNA, comprising a scaffold for binding Cas protein, a spacer sequence having one or more regions complementary to target RNA, a mismatch corresponding to adenosine or cytidine in a target RNA, wherein the guide RNA is directed specifically to a target site in an mRNA encoding Yes-associated protein 1 (YAP1) or Transcriptional co-activator with PDZ-binding motif (TAZ or WWTR1).

In some aspects, provided herein is a guide RNA, comprising a scaffold for binding Cas protein, a spacer sequence having one or more regions complementary to target RNA, a mismatch corresponding to adenosine or cytidine in a target RNA, and chemically modified bases, wherein the guide RNA is directed specifically to a target site in an mRNA encoding Yes-associated protein 1 (YAP1) or Transcriptional co-activator with PDZ-binding motif (TAZ or WWTR1).

In some aspects, provided herein is an engineered, non-naturally occurring composition for modifying a target RNA base, comprising: (a) a Cas protein, and (b) a guide RNA molecule directed to an mRNA encoding Yes-associated protein 1 (YAP1) or Transcriptional co-activator with PDZ-binding motif (TAZ or WWTR1).

In some aspects, provided herein is an engineered, non-naturally occurring composition for modifying a target RNA base, comprising: (a) a Cas protein, (b) a base editor, and (c) a guide RNA molecule directed to an mRNA encoding Yes-associated protein 1 (YAP1) or Transcriptional co-activator with PDZ-binding motif (TAZ or WWTR1).

In some aspects, provided herein is a method of modifying a target RNA base, the method comprising administering a composition comprising: (a) a Cas protein, (b) a base editor, and (c) a guide RNA molecule directed to an mRNA encoding YAP1 or TAZ, wherein the administering modifies the target RNA base.

Various aspects of the invention are described in detail in the following sections. The use of sections is not meant to limit the invention. Each section can apply to any aspect of the invention. In this application, the use of “or” means “and/or” unless stated otherwise. Reference is made herein to the following US patents and applications: Ser. Nos. 16/310,577, 16/493,464, 16/604,724, 16/617,560, 16/623,799, 16/649,170, 15/930,478, 16/650,480, 16/756,134, 16/756,139, 16/954,032, 15/482,603, 15/844,530, 16/450,699, 16/450,825, 16/450,852, 15/960,064, the contents of which are incorporated by reference herein in entirety.

### Hippo Signaling Pathway

The Hippo signaling pathway is an evolutionarily conserved kinase cascade that controls organ growth and cell proliferation by regulating the phosphorylation of transcriptional co-activators Yes-associated protein 1 (YAP1) and Transcriptional co-activator with PDZ-binding motif (TAZ or WWTR1).

The canonical pathway comprises the mammalian STE20-like kinase 1 or 2 (MST1/2) and large tumour suppressor 1 or 2 (LATS1/2) serine/threonine kinases. These kinases interact with their respective adaptor proteins, namely, SAV1 (also called WW45) and Mps one binder kinase activator-like 1 (MOB1), to phosphorylate and consequently inactivate YAP1 and TAZ. YAP1 and TAZ bind to TEA domain (TEAD) proteins 1-4 (TEAD1-4) to drive the transcription of genes involved in cell proliferation and survival. Nucleic acid (DNA) sequences of YAP1 and TAZ are as disclosed in Table 1.

TABLE 1
YAP1 and TAZ nucleic acid sequences

TAZ ORF sequence (Uniprot ID: Q9GZV5)
ATGAATCCGGCCTCGGCGCCCCCTCCGCTCCCGCCGCCTGGGCAGCAAGTGATCCACGTCACGCAGGAC
CTAGACACAGACCTCGAAGCCCTCTTCAACTCTGTCATGAATCCGAAGCCTAGCTCGTGGCGGAAGAAG
ATCCTGCCGGAGTCTTTCTTTAAGGAGCCTGATTCGGGCTCGCACTCGCGCCAGTCCAGCACCGACTCG
TCGGGCGGCCACCCGGGGCCTCGACTGGCTGGGGGTGCCCAGCATGTCCGCTCGCACTCGTCGCCCGCG
TCCCTGCAGCTGGGCACCGGCGCGGGTGCTGCGGGTAGCCCCGCGCAGCAGCACGCGCACCTCCGCCAG
CAGTCCTACGACGTGACCGACGAGCTGCCACTGCCCCCGGGCTGGGAGATGACCTTCACGGCCACTGGC
CAGAGGTACTTCCTCAATCACATAGAAAAAATCACCACATGGCAAGACCCTAGGAAGGCGATGAATCAG
CCTCTGAATCATATGAACCTCCACCCTGCCGTCAGTTCCACACCAGTGCCTCAGAGGTCCATGGCAGTA
TCCCAGCCAAATCTCGTGATGAATCACCAACACCAGCAGCAGATGGCCCCCAGTACCCTGAGCCAGCAG
AACCACCCCACTCAGAACCCACCCGCAGGGCTCATGAGTATGCCCAATGCGCTGACCACTCAGCAGCAG
CAGCAGCAGAAACTGCGGCTTCAGAGAATCCAGATGGAGAGAGAAAGGATTCGAATGCGCCAAGAGGAG
CTCATGAGGCAGGAAGCTGCCCTCTGTCGACAGCTCCCCATGGAAGCTGAGACTCTTGCCCCAGTTCAG
GCTGCTGTCAACCCACCCACGATGACCCCAGACATGAGATCCATCACTAATAATAGCTCAGATCCTTTC
CTCAATGGAGGGCCATATCATTCGAGGGAGCAGAGCACTGACAGTGGCCTGGGGTTAGGGTGCTACAGT
GTCCCCACAACTCCGGAGGACTTCCTCAGCAATGTGGATGAGATGGATACAGGAGAAAACGCAGGACAA
ACACCCATGAACATCAATCCCCAACAGACCCGTTTCCCTGATTTCCTTGACTGTCTTCCAGGAACAAAC
GTTGACTTAGGAACTTTGGAATCTGAAGACCTGATCCCCCTCTTCAATGATGTAGAGTCTGCTCTGAAC
AAAAGTGAGCCCTTTCTAACCTGGCTGTAA (SEQ ID NO: 61)
YAP ORF Sequence (variant 3) NM_001195044.2
ATGGATCCCGGGCAGCAGCCGCCGCCTCAACCGGCCCCCCAGGGCCAAGGGCAGCCGCCTTCGCAGCCC
CCGCAGGGGCAGGGCCCGCCGTCCGGACCCGGGCAACCGGCACCCGCGGCGACCCAGGCGGCGCCGCAG
GCACCCCCCGCCGGGCATCAGATCGTGCACGTCCGCGGGGACTCGGAGACCGACCTGGAGGCGCTCTTC
AACGCCGTCATGAACCCCAAGACGGCCAACGTGCCCCAGACCGTGCCCATGAGGCTCCGGAAGCTGCCC
GACTCCTTCTTCAAGCCGCCGGAGCCCAAATCCCACTCCCGACAGGCCAGTACTGATGCAGGCACTGCA
GGAGCCCTGACTCCACAGCATGTTCGAGCTCATTCCTCTCCAGCTTCTCTGCAGTTGGGAGCTGTTTCT
CCTGGGACACTGACCCCCACTGGAGTAGTCTCTGGCCCAGCAGCTACACCCACAGCTCAGCATCTTCGA
CAGTCTTCTTTTGAGATACCTGATGATGTACCTCTGCCAGCAGGTTGGGAGATGGCAAAGACATCTTCT
GGTCAGAGATACTTCTTAAATCACATCGATCAGACAACAACATGGCAGGACCCCAGGAAGGCCATGCTG
TCCCAGATGAACGTCACAGCCCCCACCAGTCCACCAGTGCAGCAGAATATGATGAACTCGGCTTCAGGT
CCTCTTCCTGATGGATGGGAACAAGCCATGACTCAGGATGGAGAAATTTACTATATAAACCATAAGAAC
AAGACCACCTCTTGGCTAGACCCAAGGCTTGACCCTCGTTTTGCCATGAACCAGAGAATCAGTCAGAGT
GCTCCAGTGAAACAGCCACCACCCCTGGCTCCCCAGAGCCCACAGGGAGGCGTCATGGGTGGCAGCAAC
TCCAACCAGCAGCAACAGATGCGACTGCAGCAACTGCAGATGGAGAAGGAGAGGCTGCGGCTGAAACAG
CAAGAACTGCTTCGGCAGGAGTTAGCCCTGCGTAGCCAGTTACCAACACTGGAGCAGGATGGTGGGACT
CAAAATCCAGTGTCTTCTCCCGGGATGTCTCAGGAATTGAGAACAATGACGACCAATAGCTCAGATCCT
TTCCTTAACAGTGGCACCTATCACTCTCGAGATGAGAGTACAGACAGTGGACTAAGCATGAGCAGCTAC
AGTGTCCCTCGAACCCCAGATGACTTCCTGAACAGTGTGGATGAGATGGATACAGGTGATACTATCAAC
CAAAGCACCCTGCCCTCACAGCAGAACCGTTTCCCAGACTACCTTGAAGCCATTCCTGGGACAAATGTG
GACCTTGGAACACTGGAAGGAGATGGAATGAACATAGAAGGAGAGGAGCTGATGCCAAGTCTGCAGGAA
GCTTTGAGTTCTGACATCCTTAATGACATGGAGTCTGTTTTGGCTGCCACCAAGCTAGATAAAGAAAGC
TTTCTTACATGGTTATAG (SEQ ID NO: 62)
YAP ORF Sequence (variant 1) NM_001130145.3
ATGGATCCCGGGCAGCAGCCGCCGCCTCAACCGGCCCCCCAGGGCCAAGGGCAGCCGCCTTCGCAGCCC
CCGCAGGGGCAGGGCCCGCCGTCCGGACCCGGGCAACCGGCACCCGCGGCGACCCAGGCGGCGCCGCAG
GCACCCCCCGCCGGGCATCAGATCGTGCACGTCCGCGGGGACTCGGAGACCGACCTGGAGGCGCTCTTC
AACGCCGTCATGAACCCCAAGACGGCCAACGTGCCCCAGACCGTGCCCATGAGGCTCCGGAAGCTGCCC
GACTCCTTCTTCAAGCCGCCGGAGCCCAAATCCCACTCCCGACAGGCCAGTACTGATGCAGGCACTGCA
GGAGCCCTGACTCCACAGCATGTTCGAGCTCATTCCTCTCCAGCTTCTCTGCAGTTGGGAGCTGTTTCT
CCTGGGACACTGACCCCCACTGGAGTAGTCTCTGGCCCAGCAGCTACACCCACAGCTCAGCATCTTCGA
CAGTCTTCTTTTGAGATACCTGATGATGTACCTCTGCCAGCAGGTTGGGAGATGGCAAAGACATCTTCT
GGTCAGAGATACTTCTTAAATCACATCGATCAGACAACAACATGGCAGGACCCCAGGAAGGCCATGCTG
TCCCAGATGAACGTCACAGCCCCCACCAGTCCACCAGTGCAGCAGAATATGATGAACTCGGCTTCAGGT
CCTCTTCCTGATGGATGGGAACAAGCCATGACTCAGGATGGAGAAATTTACTATATAAACCATAAGAAC
AAGACCACCTCTTGGCTAGACCCAAGGCTTGACCCTCGTTTTGCCATGAACCAGAGAATCAGTCAGAGT
GCTCCAGTGAAACAGCCACCACCCCTGGCTCCCCAGAGCCCACAGGGAGGCGTCATGGGTGGCAGCAAC
TCCAACCAGCAGCAACAGATGCGACTGCAGCAACTGCAGATGGAGAAGGAGAGGCTGCGGCTGAAACAG
CAAGAACTGCTTCGGCAGGCAATGCGGAATATCAATCCCAGCACAGCAAATTCTCCAAAATGTCAGGAG
TTAGCCCTGCGTAGCCAGTTACCAACACTGGAGCAGGATGGTGGGACTCAAAATCCAGTGTCTTCTCCC
GGGATGTCTCAGGAATTGAGAACAATGACGACCAATAGCTCAGATCCTTTCCTTAACAGTGGCACCTAT
CACTCTCGAGATGAGAGTACAGACAGTGGACTAAGCATGAGCAGCTACAGTGTCCCTCGAACCCCAGAT
GACTTCCTGAACAGTGTGGATGAGATGGATACAGGTGATACTATCAACCAAAGCACCCTGCCCTCACAG
CAGAACCGTTTCCCAGACTACCTTGAAGCCATTCCTGGGACAAATGTGGACCTTGGAACACTGGAAGGA
GATGGAATGAACATAGAAGGAGAGGAGCTGATGCCAAGTCTGCAGGAAGCTTTGAGTTCTGACATCCTT
AATGACATGGAGTCTGTTTTGGCTGCCACCAAGCTAGATAAAGAAAGCTTTCTTACATGGTTATAG
(SEQ ID NO: 63)

Manipulating the Hippo pathway is one approach for developing new regenerative therapies, such as for regenerating cardiomyocytes after myocardial infarction. The adult mammalian heart lacks significant regenerative potential, with injury causing irreversible scarring and fibrosis that results in high degree of mortality for patients.

In some aspects, provided herein is an engineered, non-naturally occurring composition for modifying a target RNA base, comprising: (a) a Cas protein, and (b) a guide RNA molecule directed to an mRNA encoding Yes-associated protein 1 (YAP1) or Transcriptional co-activator with PDZ-binding motif (TAZ or WWTR1).

In some aspects, provided herein is an engineered, non-naturally occurring composition for modifying a target RNA base, comprising: (a) a Cas protein, (b) a base editor, and (c) a guide RNA molecule directed to an mRNA encoding Yes-associated protein 1 (YAP1) or Transcriptional co-activator with PDZ-binding motif (TAZ or WWTR1).

Provided herein is a novel regenerative therapy for damaged myocardial tissue for treating cardiac disease using RNA editing.

In some embodiments, the modification of targeted RNA base changes one or more post-translational modification sites (e.g. phosphorylation) of the protein encoded by the target RNA, for example, changes the encoded amino acid from a serine, threonine, or tyrosine to an amino acid that cannot be phosphorylated. In some embodiments, the target RNA encodes a protein that is a transcriptional activator, co-activator or signaling protein, e.g. a protein in a kinase signaling pathway. In some embodiments, the signaling pathway is a hippo signaling pathway. In some embodiments, the target RNA encodes Yes-associated protein 1 (YAP1), for example at S127 (corresponding to mouse S112) and/or S109 (corresponding to mouse S94). In some embodiments, the target RNA base modification modifies YAP1 phosphorylation at S127. In some embodiments, the target RNA base modification modifies YAP1 phosphorylation at S109. In some embodiments, the target RNA base modification modifies YAP1 phosphorylation at S127 and/or one or more of $109, S164, S381, S383 and S384.

In some embodiments, the composition comprises a target RNA that encodes Transcriptional co-activator with PDZ-binding motif (TAZ) or WWTR1. In some embodiments, the target RNA base modification modifies TAZ phosphorylation sites at serine 89 (S89). In some embodiments, the target RNA base modification modifies TAZ phosphorylation sites at S89 and/or one or more of S314, S311, S117 and S66. In some embodiments, target RNA base modification modifies S127 on YAP1 and S89 on TAZ. In some embodiments, target RNA base modification modifies one or more YAP1 phosphorylation sites selected from S127, S109, S164, S381, S383 and S384 and one or more TAZ phosphorylation sites, selected from S89, S314, S311, S117 and S66.

In some embodiments, the composition comprises a guide RNA comprising a sequence having at least 70%, 75%, 80%, 85%, 90%, 95%, 98%, 99% or greater identity up to 100% identity to a sequence selected from the group consisting of SEQ ID NO: 34-60.

In some embodiments, the guide RNA comprises a sequence selected from the group consisting of SEQ ID NO: 34-60.

In some embodiments, the guide RNA comprises a spacer sequence having at least 70%, 75%, 80%, 85%, 90%, 95%, 98%, 99% or greater identity up to 100% identity to a sequence selected from the group consisting of SEQ ID NO: 7-33.

In some embodiments, the guide RNA comprises a spacer sequence selected from the group consisting of SEQ ID NO: 7-33.

In some embodiments, the target RNA is modified in organs selected from a group consisting of heart, liver, lung, kidney, brain, CNS or skin. In some embodiments, the target RNA is modified in the heart. In some embodiments, YAP1 RNA is modified in the heart. In some embodiments, TAZ or WWTR1 RNA is modified in the heart.

In some embodiments, the Cas protein is a catalytically inactive or dead Cas protein and the base editor is a RNA editing for programmable A-to-I replacement (REPAIR) and RNA editing for specific C-to-U exchange (RESCUE) base editor, wherein the modifying of a targeted RNA base is from adenosine to inosine or cytidine to uracil, and wherein the RNA base modification results in a modified phosphorylation site of YAP1 or TAZ protein. In some embodiments, provided herein is an engineered, non-naturally occurring system for RNA editing, comprising the compositions described herein.

In some aspects, provided herein is a method of modifying a target RNA base, the method comprising administering a composition comprising: (a) a Cas protein, (b) a base editor, and (c) a guide RNA molecule directed to an mRNA encoding YAP1 or TAZ, wherein the administering modifies the target RNA base.

In some embodiments, the method comprises administering a composition comprising: a catalytically inactive or dead Cas protein, a RESCUE or REPAIR base editor, and a guide RNA molecule; wherein the modifying of target RNA base is from adenosine to inosine or cytidine to uracil; and wherein the RNA base modification results in a modified phosphorylation site of YAP1 or TAZ protein. In some embodiments, the catalytically inactive or dead Cas protein in the method is a Type VI Cas protein, e.g. dCas13. In some embodiments, the Cas protein is PspCas13b and the base editor has at least about 80% identity to SEQ ID NO: 1 or 3. In some embodiments, the Cas protein is RanCas13b and the base editor has at least about 80% identity to SEQ ID NO: 2. In some embodiments, the Cas protein has at least about 85%, 90%, 95%, 99% or greater identity up to 100% identity to a nucleic acid sequence of SEQ ID NO: 1-3.

In some embodiments, the guide RNA comprises a sequence having at least 70%, 75%, 80%, 85%, 90%, 95%, 98%, 99% or greater identity up to 100% identity to a sequence selected from the group consisting of SEQ ID NO: 34-60.

In some embodiments, the guide RNA comprises a sequence selected from the group consisting of SEQ ID NO: 34-60.

In some embodiments, the guide RNA comprises a spacer sequence having at least 70%, 75%, 80%, 85%, 90%, 95%, 98%, 99% or greater identity up to 100% identity to a sequence selected from the group consisting of SEQ ID NO: 7-33.

In some embodiments, the guide RNA comprises a spacer sequence selected from the group consisting of SEQ ID NO: 7-33.

In some embodiments, the modification of a targeted RNA base, changes the post-translational modification of the encoded protein. In some embodiments, the modification of a targeted RNA base, changes the serine 127 (S127) of YAP1 protein to an amino acid that cannot be phosphorylated. In some embodiments, the modification of a targeted RNA base, changes the serine 109 (S109) of YAP1 protein to an amino acid that cannot be phosphorylated, thereby activating YAP1. In some embodiments, the modification of a targeted RNA base, changes S127 and one or more YAP1 phosphorylation sites selected from S109, S164, S381, S383 and S384 to an amino acid that cannot be phosphorylated, thereby activating YAP1. In some embodiments, the modification of a targeted RNA base, changes S89 or TAZ protein to an amino acid that cannot be phosphorylated, thereby activating TAZ. In some embodiments, the modification of a targeted RNA base, changes S89 and one or more TAZ phosphorylation sites selected from S314, S311, S117 and S66 to an amino acid that cannot be phosphorylated, thereby activating TAZ. In some embodiments, the modification of a targeted RNA base changes one or more YAP1 phosphorylation sites selected from S127, S109, S164, S381, S383 and S384 and/or one or more TAZ phosphorylation sites, selected from S89, S314, S311, S117 and S66 to an amino acid that cannot be phosphorylated, thereby activating YAP1 and/or TAZ.

In some embodiments, the method leads to an increase or decrease in expression of a target RNA.

In some embodiments, provided herein is a method of treating disease by administering to a subject in need thereof, an effective amount of the composition disclosed herein, wherein the composition activates or inactivates a signaling pathway by a post-translational modification, e.g. phosphorylation. In some embodiments, the disease is caused by activation of a kinase pathway.

In some embodiments, the disease is a degenerative disease. In some embodiments, the disease affects one or more organs from heart, lung, liver, kidney, brain, CNS or skin. In some embodiments, the disease is a cardiac disease. In some embodiments, the disease is caused by phosphorylation of YAP1 protein at S127 or S109. In some embodiments, the disease is caused by phosphorylation at one or more YAP1 phosphorylation sites selected from S127, S109, S164, S381, S383 and S384 to an amino acid that cannot be phosphorylated, thereby activating YAP1. In some embodiments, the disease is caused by phosphorylation of TAZ protein. In some embodiments, the disease is caused by phosphorylation at S89. In some embodiments, the disease is caused by phosphorylation at one or more TAZ phosphorylation sites selected from S89, S314, S311, S117 and S66 to an amino acid that cannot be phosphorylated, thereby activating TAZ. In some embodiments, the disease is caused by phosphorylation at one or more of YAP1 or TAZ phosphorylation sites. In some embodiments, the disease is caused by phosphorylation at one or more YAP1 phosphorylation sites selected from S127, S109, S164, S381, S383 and S384 and/or one or more TAZ phosphorylation sites, selected from S89, S314, S311, S117 and S66 to an amino acid that cannot be phosphorylated, thereby activating YAP1 and/or TAZ.

In some embodiments, the administering of the composition deaminates adenosine to inosine or cytosine to uracil, thereby mutating a phosphorylation site and preventing phosphorylation of YAP1 or TAZ.

In some embodiments, the administering of the composition in vivo is at a molar ratio of base editor:guide RNA of between about 1:1 to 1:50. In some embodiments, the administering of the composition in vivo is at a molar ratio of base editor:guide RNA of about 1:1, 1:5, 1:10, 1:15, 1:20, 1:25, 1:30, 1:35, 1:40, 1:45, or 1:50. In some embodiments, the administering of the composition increases replication, organ size growth, stem cell renewal and cell survival. In some embodiments, the administering of the composition to cardiac tissue or cardiomyocytes results in decreased scarring or fibrosis.

### RNA Editing

RNA editing is an evolutionarily conserved molecular process by which cells edit specific nucleotide sequences within an RNA molecule after transcription by an RNA polymerase.

Directing ADAR to specific sites on mRNA to correct genetic mutations or alter post-translational modification sites, thereby altering protein expression and activity has therapeutic applications in treating disease. Due to the transient nature of RNA, RNA editing is also useful for treating non-genetic diseases caused by temporary changes in cell state, such as local inflammation, and diseases caused by RNA-level defects such as splicing variants. Editing splicing forms, disrupting RNA-RNA base pairing or eliminating toxic RNA by RNA editing has therapeutic advantages over DNA editing. RNA editing can also be used to treat disorders caused by modification of proteins involved in disease-related signal transduction.

In some embodiments, RNA base editing is performed by an endogenous deaminase enzyme recruited by guide RNA to target RNA. In some embodiments, RNA editing is carried out by a deaminase that is provided or acts in trans, i.e. which is not fused to a Cas protein. In some embodiments, base editing is performed by an exogenous deaminase enzyme. In some embodiments, RNA editing is carried out by a programmable RNA binding protein (e.g. Type VI Cas, e.g. a catalytically inactive or dead Cas, e.g. dCas13) fused to a deaminase (e.g. adenosine or adenine deaminase or cytidine or cytosine deaminase), i.e. in cis. In some embodiments, RNA editing is carried out by a Cas protein fused to an ADAR. In some embodiments, RNA editing is carried out by a Cas protein fused to a cytidine or cytosine deaminase. In some embodiments, RNA editing is carried out by a Cas protein fused to an adenosine or adenine deaminase.

The present invention provides, among other things, for example, guide RNAs, compositions and methods for altering the phosphorylation status of YAP1 and TAZ proteins, for use in treating disease, for example, cardiac disease.

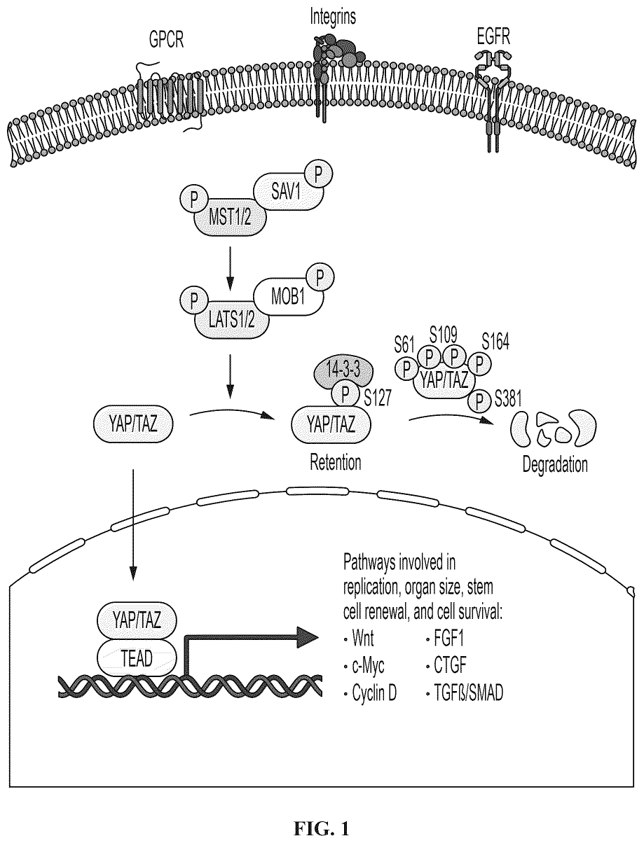

Without wishing to be bound to any particular theory, it is contemplated that the YAP1 protein when phosphorylated at for example, serine 127, and subsequently S109, S164, S381, S383 and S384 in the cytosol, the YAP1 protein is targeted for degradation. When YAP1 protein is non-phosphorylated (e.g. due to an amino acid change from RNA editing that disrupts the phosphorylation site), YAP1 enters the nucleus and interacts with the TEAD family of transcription factors to activate transcription of pathways involved in replication, organ size, stem cell renewal and cell survival such as, but not limited to Wnt, c-Myc, cyclin D, FGF1, CTGF, TGFβ/SMAD (FIG. 1).

Similarly, in TAZ, serine 89 is phosphorylated, and subsequently S62, S295, and S311 are phosphorylated. Phosphorylation of TAZ results in retention in the cytoplasm, and functional inactivation. Phosphorylation results in the inhibition of transcriptional coactivation through YWHAZ-mediated nuclear export.

In some embodiments, the composition comprises a Cas protein, wherein the Cas protein is a Type VI Cas protein. In some embodiments, the composition comprises a Cas protein, wherein the Cas protein is Prevotella sp. PspCas13b or Riemerella anatipestifer RanCas13b. In some embodiments, the composition comprises a Cas protein, wherein the Cas protein is a catalytically inactive Cas13 or dead Cas13 (dCas13). In some embodiments, the composition comprises a base editor that comprises one or more RNA binding domains fused to a nucleotide deaminase. In some embodiments, the composition comprises a base editor, wherein the base editor is ADAR or active fragment thereof. In some embodiments, the base editor is ADAR2 or active fragment thereof. In some embodiments, the deaminase is exogenous. In some embodiments, the deaminase is endogenous.

In some embodiments, the composition comprises a base editor, wherein the base editor comprises a deaminase domain fused by a linker to catalytically inactive or dCas13, and a nuclear export signal. In some embodiments, the composition comprises a deaminase, wherein the deaminase is fused to an N-terminus of dCas13. In some embodiments, the composition comprises a deaminase, wherein the deaminase is fused to a C-terminus of dCas13.

In some embodiments, the composition comprises a base editor, wherein the base editor is an adenine deaminase. In some embodiments, the composition comprises a base editor, wherein the base editor is a REPAIR editor. In some embodiments, the composition comprises a base editor, wherein the base editor deaminates adenosine to inosine (A to I).

In some embodiments, the composition comprises a base editor, wherein the base editor is a cytosine deaminase. In some embodiments, the composition comprises a base editor, wherein the base editor is a RESCUE editor. In some embodiments, the composition comprises a base editor, wherein the base editor deaminates cytidine to uridine (C to U) and/or adenine to inosine.

In some embodiments, the composition comprises a base editor, wherein the base editor has at least about 80% identity to a nucleic acid sequence of SEQ ID NO: 1-3. In some embodiments, the composition comprises a base editor, wherein the base editor has at least about 85%, 90%, 95%, 99% or greater identity up to 100% identity to a nucleic acid sequence of SEQ ID NO: 1-3.

A phosphodegron is a one or a series of phosphorylated residues on the substrate that directly interact with a protein-protein interaction domain in an E3 Ub-ligase, thereby linking the substrate to the conjugation machinery. In some embodiments, the phosphodegron is a repetition of amino acids in the YAP1 or TAZ mRNA that has a pattern, HXRXXS (X is any amino acid; H is histidine; R is arginine and S is serine). In various embodiments, all serines in a phosphodegron are targeted independently in combination with S89 in TAZ or S127 in YAP. In some embodiments, RNA editing is multiplexed in some serine residues in the phosphodegron to promote activation of YAP1 or TAZ.

In some embodiments, Cas proteins of the Type VI family are used to direct endogenous or exogenous ADARs or the catalytic domain of engineered ADARs to RNA using guide RNAs. In some embodiments, the Cas protein is a deadCas. In some embodiments, the Cas protein is a Cas13. In some embodiments, the Cas protein is a deadCas13.

### Guide RNAs Targeting YAP1 and TAZ Transcriptional Co-Activators in the Hippo Pathway

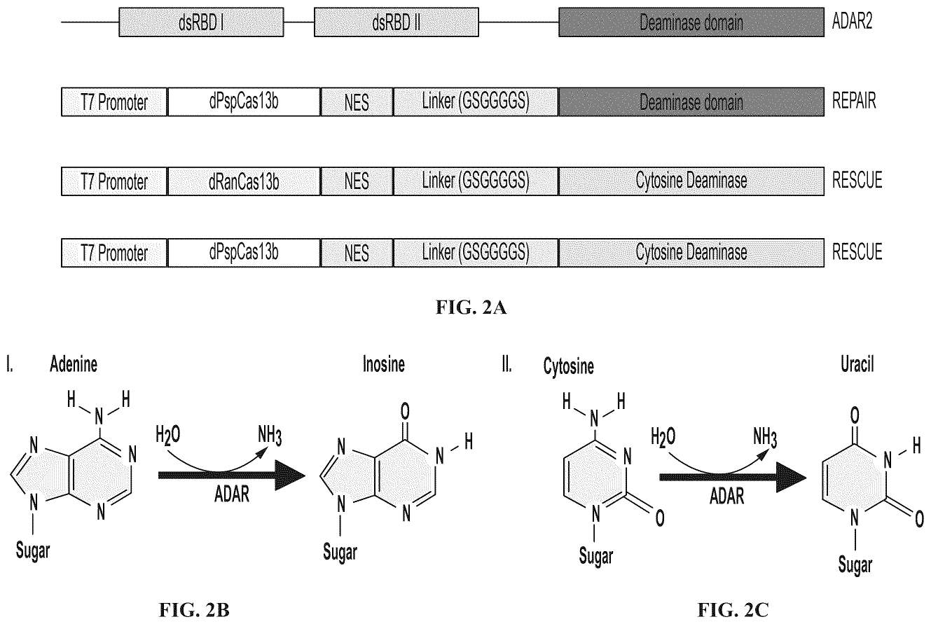

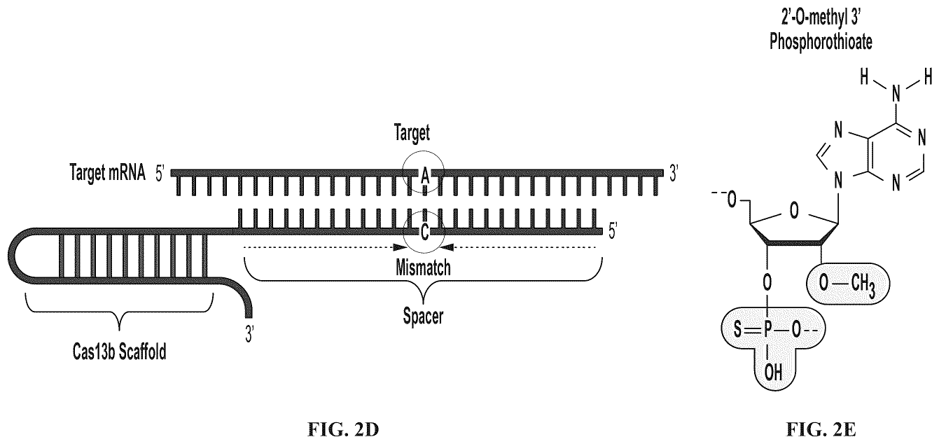

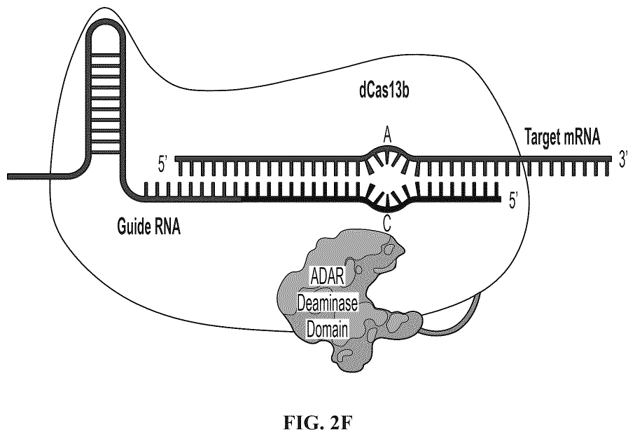

Guide RNAs are designed targeting YAP1 and TAZ transcriptional co-activators in the Hippo pathway. In some aspects, provided herein is a guide RNA, comprising a scaffold for binding Cas protein, a spacer sequence having one or more regions complementary to target RNA, a mismatch corresponding to adenosine or cytidine in a target RNA, wherein the guide RNA is directed specifically to a target site in an mRNA encoding Yes-associated protein 1 (YAP1) or Transcriptional co-activator with PDZ-binding motif (TAZ or WWTR1). For example, guide RNA with a Cas13b scaffold, and a spacer recognizes mRNA comprising an A-C mismatch (FIG. 2D). Guide RNA recruits Cas13b fused to ADAR to a specific site on mRNA (FIG. 2F).

The guide RNAs comprise a spacer length of between 30 nucleotides and 36 nucleotides. In some embodiments, there is a mismatch 17, 24, 25 to 26 nucleotides from the scaffold within the spacer. In some embodiments, there is a 3′ universal extension piece (6 nt) added to all the guides: UUmC*mG*mA*U (SEQ ID NO: 4). In some embodiments, RNA comprises chemical modifications at 5′ and/or 3′ end. E.g. 3× 2′-O-methyl 3′ phosphorothioate and phosphorothioate (FIG. 2E). The guide RNAs comprise a scaffold with extension sequence, e.g.

Psp scaffold:
(SEQ ID NO: 5)
GUUGUGGAAGGUCCAGUUUUGAGGGGCUAUUACAAC,
and
Ran scaffold:
(SEQ ID NO: 6)
GUUGGGACUGCUCUCACUUUGAAGGGUAUUCACAAC.

In some embodiments, the guide RNA comprises a spacer comprising a sequence having at least 70%, 75%, 80%, 85%, 90%, 95% or greater identity up to 100% identity to SEQ ID NO: 7-33. In some embodiments, the guide RNA comprises a spacer comprising a sequence of any one of SEQ ID NO: 7-33. In some embodiments, the guide RNA comprises a sequence having at least 70%, 75%, 80%, 85%, 90%, 95% or greater identity up to 100% identity to any one of SEQ ID NO: 34-60. In some embodiments, the guide RNA comprises a sequence of any one of SEQ ID NO: 34-60.

In some embodiments, the guide RNA comprising a spacer complementary to YAP1 is any one of SEQ ID NO: 7, 8, 22-33. In some embodiments, the guide RNA comprising a spacer complementary to TAZ or WWTR1 is any one of SEQ ID NO: 9-21.

TABLE 2
Guide RNA spacer sequences targeting YAP1 and TAZ (seq 5′ to 3′)

| | | |
|---|---|---|
| SG- | YAPS127F- | mA*mG*mA*GUAAUGAGCUCGAACAUGCUGUGGA |
| MM137 | 30ntMM26-U | GU (SEQ ID NO: 7) |
| spacer | MM05 Psp | |
| | equivalent to MM05 | |
| | for Psp-RESCUE | |
| | spacer | |
| SG- | YAPS127F- | mA*mG*mA*AGCUGGAGAGUAAUGAGCUCGAACA |
| MM138 | 30ntMM17-U Psp | UG (SEQ ID NO: 8) |
| spacer | equivalent MM03 | |
| | for Psp-RESCUE | |
| | spacer | |
| SG- | TAZg24_S314 | mA*mA*mC*CCCAGGCCACcGUCAGUGCUCUGCUC |
| MM139 | MM17-C spacer | C (SEQ ID NO: 9) |
| spacer | | |
| SG- | TAZg25_S311_S31 | mG*mG*mC*CACcGUCAGUGCcCUGCUCCCUCGAA |
| MM140 | 4 MM24-15 spacer | U (SEQ ID NO: 10) |
| spacer | | |
| SG- | TAZg26_S311_S31 | mC*mC*mA*CcGUCAGUGCcCUGCUCCCUCGAAUG |
| MM141 | 4 MM26-17 spacer | A (SEQ ID NO: 11) |
| spacer | | |
| SG- | TAZg27_S311_S31 | mU*mA*mA*CCCCAGGCCACcGUCAGUGCcCUGCU |
| MM142 | 4 MM26-17_40nt | CCCUCGAAUGA (SEQ ID NO: 12) |
| spacer | spacer | |
| SG- | TAZg16_S117 | mG*mG*mU*CACGUCGUAGuACUGCUGGCGGAGG |
| MM143 | MM17-U spacer | UG (SEQ ID NO: 13) |
| spacer | | |
| SG- | TAZg20_S117 | mU*mC*mG*UAGuACUGCUGGCGGAGGUGCGCGU |
| MM144 | MM24-U spacer | GC (SEQ ID NO: 14) |
| spacer | | |
| SG- | TAZg23_S117 | mG*mU*mA*GuACUGCUGGCGGAGGUGCGCGUGC |
| MM145 | MM26-U spacer | UG (SEQ ID NO: 15) |
| spacer | | |
| SG- | TAZg1_S66_MM17 | mG*mA*mC*GAGUCGGUGCcGGACUGGCGCGAGU |
| MM146 | spacer | GC (SEQ ID NO: 16) |
| spacer | | |
| SG- | TAZg3_S66_MM24 | mC*mG*mG*UGCcGGACUGGCGCGAGUGCGAGCC |
| MM147 | spacer | CG (SEQ ID NO: 17) |
| spacer | | |
| SG- | TAZg5_S66_MM26 | mG*mU*mG*CcGGACUGGCGCGAGUGCGAGCCCG |
| MM148 | spacer | AA (SEQ ID NO: 18) |
| spacer | | |
| SG- | TAZg9_S89_MM17-U | mG*mG*mA*CGCGGGCGACuAGUGCGAGCGGACA |
| MM149 | spacer | UG (SEQ ID NO: 19) |
| spacer | | |
| SG- | TAZg11_S89 | mG*mG*mC*GACuAGUGCGAGCGGACAUGCUGGG |
| MM150 | MM24-U spacer | CA (SEQ ID NO: 20) |
| spacer | | |
| SG- | TAZg13_S89 | mC*mG*mA*CuAGUGCGAGCGGACAUGCUGGGCA |
| MM151 | MM26-U spacer | CC_(SEQ ID NO: 21) |
| spacer | | |
| SG- | YAP S164 | mC*mA*mA*AAUAAGACUGUCGAAGAUGCUGAGC |
| MM152 | 30ntMM25-U spacer | UG (SEQ ID NO: 22) |
| spacer | | |
| SG- | YAP S164 | mC*mA*mA*AAcAAGACUGUCGAAGAUGCUGAGC |
| MM153 | 30ntMM25-C spacer | UG (SEQ ID NO: 23) |
| spacer | | |
| SG- | YAP S109- | mC*mC*mU*GCAUCAGUACcGGCCUGUCGGGAGU |
| MM60 | 30ntMM17- | GG (SEQ ID NO: 24) |
| spacer | REPAIR_COR | |
| | spacer | |
| SG- | YAP S109- | mA*mG*mU*ACcGGCCUGUCGGGAGUGGGAUUUG |
| MM61 | 30ntMM25- | GG (SEQ ID NO: 25) |
| spacer | REPAIR_COR | |
| | spacer | |
| sg- | YAPS381G- | mA*mA*mA*GGAUCUGAGCcAUUGGUCGUCAUUG |
| MM214 | 30ntMM17-REPAIR | UU |
| spacer | spacer | (SEQ ID NO: 26) |
| sg- | YAPS381G- | mU*mG*mA*GCcAUUGGUCGUCAUUGUUCUCAAU |
| MM215 | 30ntMM25-REPAIR | UC (SEQ ID NO: 27) |
| spacer | spacer | |
| sg- | YAPS381G- | mG*mU*mA*CcCUCAUCUCGAGAGUGAUAGGUGC |
| MM216 | 30ntMM26-REPAIR | CA (SEQ ID NO: 28) |
| spacer | spacer | |
| sg- | YAPS381G- | mC*mU*mG*UCUGUACcCUCAUCUCGAGAGUGAU |
| MM217 | 36ntMM26-REPAIR | AGGUGCCA (SEQ ID NO: 29) |
| spacer | spacer | |
| sg- | YAPS384G- | mA*mU*mG*CUUAGUCCACcGUCUGUACUCUCAU |
| MM218 | 30ntMM17-REPAIR | CU (SEQ ID NO: 30) |
| spacer | spacer | |
| sg- | YAPS384G- | mC*mU*mU*AGUCCACcGUCUGUACUCUCAUCUC |
| MM219 | 36ntMM26-REPAIR | GAGAGUGA (SEQ ID NO: 31) |
| spacer | spacer | |
| sg- | YAPD383G- | mG*mC*mU*UAGUCCACUGcCUGUACUCUCAUCU |
| MM220 | 30ntMM17-REPAIR | CG (SEQ ID NO: 32) |
| spacer | spacer | |
| sg- | YAPD383G- | mU*mA*mG*UCCACUGcCUGUACUCUCAUCUCGA |
| MM221 | 36ntMM26-REPAIR | GAGUGAUA (SEQ ID NO: 33) |
| spacer | spacer | |

Mismatches (bold, underlined). RESCUE editor systems predominantly comprise U mismatches; however, in some embodiments, C mismatches are present. REPAIR editor systems predominantly comprise C mismatches.

TABLE 3
Guide RNA sequences targeting YAP1 and TAZ (seq 5′ to 3′) including
spacer, scaffold and extension sequences

| | | |
|---|---|---|
| SG- | YAPS127F- | mA*mG*mA*GUAAUGAGCUCGAACAUGCUGUGGA |
| MM137 | 30ntMM26-U | GUGUUGUGGAAGGUCCAGUUUUGAGGGGCUAUU |
| guide | MM05 Psp | ACAACUUmC*mG*mA*U (SEQ ID NO: 34) |
| | equivalent to | |
| | MM05 for Psp- | |
| | RESCUE guide | |
| SG- | YAPS127F- | mA*mG*mA*AGCUGGAGAGUAAUGAGCUCGAACA |
| MM138 | 30ntMM17-U Psp | UGGUUGUGGAAGGUCCAGUUUUGAGGGGCUAUU |
| guide | equivalent MM03 | ACAACUUmC*mG*mA*U (SEQ ID NO: 35) |
| | for Psp-RESCUE | |
| | guide | |
| SG- | TAZg24_S314 | mA*mA*mC*CCCAGGCCACcGUCAGUGCUCUGCUC |
| MM139 | MM17-C guide | CGUUGUGGAAGGUCCAGUUUUGAGGGGCUAUUA |
| guide | | CAACUUmC*mG*mA*U (SEQ ID NO: 36) |
| SG- | TAZg25_S311_S314 | mG*mG*mC*CACcGUCAGUGCcCUGCUCCCUCGAA |
| MM140 | MM24-15 guide | UGUUGUGGAAGGUCCAGUUUUGAGGGGCUAUUA |
| guide | | CAACUUmC*mG*mA*U (SEQ ID NO: 37) |
| SG- | TAZg26_S311_S314 | mC*mC*mA*CcGUCAGUGCcCUGCUCCCUCGAAUG |
| MM141 | MM26-17 guide | AGUUGUGGAAGGUCCAGUUUUGAGGGGCUAUUA |
| guide | | CAACUUmC*mG*mA*U (SEQ ID NO: 38) |
| SG- | TAZg27_S311_S314 | mU*mA*mA*CCCCAGGCCACcGUCAGUGCcCUGCU |
| MM142 | MM26-17_40nt | CCCUCGAAUGAGUUGUGGAAGGUCCAGUUUUGA |
| guide | guide | GGGGCUAUUACAACUUmC*mG*mA*U (SEQ ID NO: 39) |
| SG- | TAZg16_S117 | mG*mG*mU*CACGUCGUAGuACUGCUGGCGGAGG |
| MM143 | MM17-U guide | UGGUUGUGGAAGGUCCAGUUUUGAGGGGCUAUU |
| guide | | ACAACUUmC*mG*mA*U (SEQ ID NO: 40) |
| SG- | TAZg20_S117 | mU*mC*mG*UAGuACUGCUGGCGGAGGUGCGCGU |
| MM144 | MM24-U guide | GCGUUGUGGAAGGUCCAGUUUUGAGGGGCUAUU |
| guide | | ACAACUUmC*mG*mA*U (SEQ ID NO: 41) |
| SG- | TAZg23_S117 | mG*mU*mA*GuACUGCUGGCGGAGGUGCGCGUGC |
| MM145 | MM26-U guide | UGGUUGUGGAAGGUCCAGUUUUGAGGGGCUAUU |
| guide | | ACAACUUmC*mG*mA*U (SEQ ID NO: 42) |
| SG- | TAZg1_S66_MM17 | mG*mA*mC*GAGUCGGUGCcGGACUGGCGCGAGU |
| MM146 | guide | GCGUUGUGGAAGGUCCAGUUUUGAGGGGCUAUU |
| guide | | ACAACUUmC*mG*mA*U (SEQ ID NO: 43) |
| SG- | TAZg3_S66_MM24 | mC*mG*mG*UGCcGGACUGGCGCGAGUGCGAGCC |
| MM147 | guide | CGGUUGUGGAAGGUCCAGUUUUGAGGGGCUAUU |
| guide | | ACAACUUmC*mG*mA*U (SEQ ID NO: 44) |
| SG- | TAZg5_S66_MM26 | mG*mU*mG*CcGGACUGGCGCGAGUGCGAGCCCG |
| MM148 | guide | AAGUUGUGGAAGGUCCAGUUUUGAGGGGCUAUU |
| guide | | ACAACUUmC*mG*mA*U (SEQ ID NO: 45) |
| SG- | TAZg9_S89_MM17-U | mG*mG*mA*CGCGGGCGACuAGUGCGAGCGGACA |
| MM149 | guide | UGGUUGUGGAAGGUCCAGUUUUGAGGGGCUAUU |
| guide | | ACAACUUmC*mG*mA*U (SEQ ID NO: 46) |
| SG- | TAZg11_S89 | mG*mG*mC*GACuAGUGCGAGCGGACAUGCUGGG |
| MM150 | MM24-U guide | CAGUUGUGGAAGGUCCAGUUUUGAGGGGCUAUU |
| guide | | ACAACUUmC*mG*mA*U (SEQ ID NO: 47) |
| SG- | TAZg13_S89 | mC*mG*mA*CuAGUGCGAGCGGACAUGCUGGGCA |
| MM151 | MM26-U guide | CCGUUGUGGAAGGUCCAGUUUUGAGGGGCUAUU |
| guide | | ACAACUUmC*mG*mA*U (SEQ ID NO: 48) |
| SG- | YAP S164 | mC*mA*mA*AAUAAGACUGUCGAAGAUGCUGAGC |
| MM152 | 30ntMM25-U guide | UGGUUGUGGAAGGUCCAGUUUUGAGGGGCUAUU |
| guide | | ACAACUUmC*mG*mA*U (SEQ ID NO: 49) |
| SG- | YAP S164 | mC*mA*mA*AAcAAGACUGUCGAAGAUGCUGAGC |
| MM153 | 30ntMM25-C guide | UGGUUGUGGAAGGUCCAGUUUUGAGGGGCUAUU |
| guide | | ACAACUUmC*mG*mA*U (SEQ ID NO: 50) |
| SG- | YAP S109- | mC*mC*mU*GCAUCAGUACcGGCCUGUCGGGAGU |
| MM60 | 30ntMM17- | GGGUUGUGGAAGGUCCAGUUUUGAGGGGCUAUU |
| guide | REPAIR_COR | ACAACUUmC*mG*mA*U (SEQ ID NO: 51) |
| | guide | |
| SG- | YAP S109- | mA*mG*mU*ACcGGCCUGUCGGGAGUGGGAUUUG |
| MM61 | 30ntMM25- | GGGUUGUGGAAGGUCCAGUUUUGAGGGGCUAUU |
| guide | REPAIR_COR | ACAACUUmC*mG*mA*U (SEQ ID NO: 52) |
| | guide | |
| sg- | YAPS381G- | mA*mA*mA*GGAUCUGAGCcAUUGGUCGUCAUUG |
| MM214 | 30ntMM17- | UUGUUGUGGAAGGUCCAGUUUUGAGGGGCUAUUAC |
| guide | REPAIR guide | AACUUmC*mG*mA*U (SEQ ID NO: 53) |
| sg- | YAPS381G- | mU*mG*mA*GCcAUUGGUCGUCAUUGUUCUCAAU |
| MM215 | 30ntMM25- | UCGUUGUGGAAGGUCCAGUUUUGAGGGGCUAUUAC |
| guide | REPAIR guide | AACUUmC*mG*mA*U (SEQ ID NO: 54) |
| sg- | YAPS381G- | mG*mU*mA*CcCUCAUCUCGAGAGUGAUAGGUGC |
| MM216 | 30ntMM26- | CAGUUGUGGAAGGUCCAGUUUUGAGGGGCUAUUAC |
| guide | REPAIR guide | AACUUmC*mG*mA*U (SEQ ID NO: 55) |
| sg- | YAPS381G- | mC*mU*mG*UCUGUACcCUCAUCUCGAGAGUGAU |
| MM217 | 36ntMM26- | AGGUGCCAGUUGUGGAAGGUCCAGUUUUGAGGGGCUAUUAC |
| guide | REPAIR guide | AACUUmC*mG*mA*U (SEQ ID NO: 56) |
| sg- | YAPS384G- | mA*mU*mG*CUUAGUCCACcGUCUGUACUCUCAU |
| MM218 | 30ntMM17- | CUGUUGUGGAAGGUCCAGUUUUGAGGGGCUAUUAC |
| guide | REPAIR guide | AACUUmC*mG*mA*U (SEQ ID NO: 57) |
| sg- | YAPS384G- | mC*mU*mU*AGUCCACcGUCUGUACUCUCAUCUC |
| MM219 | 36ntMM26- | GAGAGUGAGUUGUGGAAGGUCCAGUUUUGAGGGGCUAUUAC |
| guide | REPAIR guide | AACUUmC*mG*mA*U (SEQ ID NO: 58) |
| sg- | YAPD383G- | mG*mC*mU*UAGUCCACUGcCUGUACUCUCAUCU |
| MM220 | 30ntMM17- | CGGUUGUGGAAGGUCCAGUUUUGAGGGGCUAUUAC |
| guide | REPAIR guide | AACUUmC*mG*mA*U (SEQ ID NO: 59) |
| sg- | YAPD383G- | mU*mA*mG*UCCACUGcCUGUACUCUCAUCUCGA |
| MM221 | 36ntMM26- | GAGUGAUAGUUGUGGAAGGUCCAGUUUUGAGGGGCUAUUAC |
| guide | REPAIR guide | AACUUmC*mG*mA*U (SEQ ID NO: 60) |

Mismatches (bold, underlined). RESCUE editor systems predominantly comprise U mismatches; however, in some embodiments, C mismatches are present. REPAIR editor systems predominantly comprise C mismatches.

In some embodiments, the spacer comprises between about 4-15 consecutive nucleotides that are perfectly complementary to the target mRNA prior to the mismatch within the spacer. In some embodiments, the spacer comprises between about 16-25 consecutive nucleotides that are perfectly complementary to the target mRNA after the mismatch within the spacer. In some embodiments, the spacer comprises between about 28-35 nonconsecutive nucleotides that are perfectly complementary to the target mRNA.

In some embodiments, the spacer sequence comprises a sequence selected from the group consisting of SEQ ID NO: 7-SEQ ID NO: 33.

In some embodiments, the guide RNA is bound to a base editor. In some embodiments, the guide RNA is bound to an mRNA encoding YAP1 or TAZ. In some embodiments, the guide RNA is bound to an mRNA encoding YAP1. In some embodiments, the guide RNA is bound to an mRNA encoding TAZ. In some embodiments, the guide RNA comprises a region that binds to an ADAR protein. In some embodiments, the guide RNA recruits endogenous ADAR to a target RNA site for base editing.

In some embodiments, the scaffold is capable of binding RESCUE or REPAIR base editor and directs it to a target site. In some embodiments, the scaffold is capable of binding RESCUE base editor and directs it to a target site. In some embodiments, the scaffold is capable of binding REPAIR base editor and directs it to a target site.

In some embodiments, the guide RNA is used to target a Cas protein fused to a base editor to YAP1 or TAZ mRNA. In some embodiments, the guide RNA is used to target a Cas protein fused to a base editor to YAP1 mRNA. In some embodiments, the guide RNA is used to target a Cas protein fused to a base editor to TAZ mRNA.

In some embodiments, the guide RNA directs a Cas protein and a RESCUE or REPAIR base editor to a target site. In some embodiments, the guide RNA directs a Cas protein and a RESCUE base editor to a target site. In some embodiments, the guide RNA directs a Cas protein and a REPAIR base editor to a target site. In some embodiments, the guide RNA directs a Cas protein and a deaminase enzyme to a target site.

In some embodiments, the guide RNA comprises a spacer of between about 30-36 nucleotides. In some embodiments, the guide RNA comprises a spacer of about 30 nucleotides. In some embodiments, the guide RNA comprises a spacer of about 31 nucleotides. In some embodiments, the guide RNA comprises a spacer of about 32 nucleotides. In some embodiments, the guide RNA comprises a spacer of about 33 nucleotides. In some embodiments, the guide RNA comprises a spacer of about 34 nucleotides. In some embodiments, the guide RNA comprises a spacer of about 35 nucleotides. In some embodiments, the guide RNA comprises a spacer of about 36 nucleotides.

In some embodiments, the guide RNA comprises a mismatch about 17, 24, 25 or 26 nucleotides in length from the scaffold. In some embodiments, the guide RNA comprises a mismatch about 17 nucleotides in length from the scaffold. In some embodiments, the guide RNA comprises a mismatch about 18 nucleotides in length from the scaffold. In some embodiments, the guide RNA comprises a mismatch about 19 nucleotides in length from the scaffold. In some embodiments, the guide RNA comprises a mismatch about 20 nucleotides in length from the scaffold. In some embodiments, the guide RNA comprises a mismatch about 21 nucleotides in length from the scaffold. In some embodiments, the guide RNA comprises a mismatch about 22 nucleotides in length from the scaffold. In some embodiments, the guide RNA comprises a mismatch about 23 nucleotides in length from the scaffold. In some embodiments, the guide RNA comprises a mismatch about 24 nucleotides in length from the scaffold. In some embodiments, the guide RNA comprises a mismatch about 25 nucleotides in length from the scaffold. In some embodiments, the guide RNA comprises a mismatch about 26 nucleotides in length from the scaffold.

In some embodiments, the guide RNA comprises chemical modifications at 5′ and/or 3′ end. In some embodiments, the guide RNA comprises 3× 2′O-methyl and/or phosphorothioate at 5′ and/or 3′ end. In some embodiments, the guide RNA comprises a 6 nucleotide extension sequence. In some embodiments, the extension sequence comprises UUmC*mG*mA*U (SEQ ID NO: 4). The annotations mC*mG*mA*U refer to chemically modified bases, for example mC* refers to modified cytosine, mG* refers to modified guanine and mA* refers to modified adenine. The m annotations followed by a base and * signifies where the base is modified by a 2′O-methyl (e.g., mC* or mG*). The * annotation between two bases signifies a 2′O-methyl phosphothiode linkage.

In some embodiments, the scaffold sequence is derived from Prevotella sp. or Riemerellar anatipestifer. In some embodiments, the scaffold sequence is a Prevotella sp. scaffold of SEQ ID NO: 5. In some embodiments, the scaffold sequence is a Riemerella anatipestifer scaffold of SEQ ID NO: 6.

An RNA guide comprises a polynucleotide sequence with complementarity to a target sequence. The RNA guide hybridizes with the target nucleic acid sequence and directs sequence-specific binding of a CRISPR complex to the target nucleic acid. In some embodiments, an RNA guide has 50%, 60%, 70%, 75%, 80%, 85%, 90%, 95%, 96%, 97%, 98%, 99% or greater identity up to 100% identity complementarity to a target nucleic acid sequence.

In some embodiments, the RNA guides are about 5, 10, 11, 12, 13, 14, 15, 16, 17, 18, 19, 20, 21, 22, 23, 24, 25, 26, 27, 28, 29, 30, 35, 40, 45, 50, 75 or more nucleotides in length. In some embodiments, the RNA guides are about 18-24 nucleotides in length. In some embodiments, the RNA guide is complementary to about 18-24 nucleotides in the target nucleic acid sequence. For example, the RNA guide is complementary to about 18, 19, 20, 21, 22, 23, or 24 nucleotides in the target nucleic acid sequence. In some embodiments, the RNA guide is complementary to about 18-22 nucleotides. In some embodiments, the RNA guide is complementary to about 18-21 nucleotides. In some embodiments, the RNA guide is complementary to about 18-20 nucleotides. In some embodiments, the RNA guide is complementary to 20 nucleotides in the target nucleic acid sequence.

An RNA guide can be designed to target any target sequence. Optimal alignment is determined using any algorithm for aligning sequences, including the Needleman-Wunsch algorithm, Smith-Waterman algorithm, Burrows-Wheeler algorithm, ClustlW, ClustlX, BLAST, Novoalign, SOAP, Maq, and ELAND.

In some embodiments, an RNA guide is targeted to a unique target sequence within the genome of a cell. In some embodiments, an RNA guide is designed to lack a protospacer adjacent motif (PAM) sequence. In some embodiments, an RNA guide sequence is designed to have optimal secondary structure using a folding algorithm including mFold or Geneious. In some embodiments, expression of RNA guides may be under an inducible promoter, e.g. hormone inducible, tetracycline or doxycycline inducible, arabinose inducible, or light inducible.

In some embodiments, the CRISPR system includes one or more RNA guides e.g. crRNA, tracrRNA, and/or sgRNA. Accordingly, in some embodiments the RNA guide comprises a crRNA. In some embodiments, the RNA guide comprises a tracrRNA. In some embodiments, the RNA guide comprises a sgRNA. In some embodiments, the CRISPR system includes multiple RNA guides, comprising 1, 2, 3, 4, 5, 6, 7, 8, 9, 10, 15 or more RNA guides.

In some embodiments, the RNA guide includes a crRNA. In some embodiments, the CRISPR system includes multiple crRNAs comprising 2-15 crRNAs. In some embodiments, the crRNA is a precursor crRNA (pre-crRNA), which includes a direct repeat sequence, a spacer sequence and a direct repeat sequence. In some embodiments, the crRNA is a processed or mature crRNA which includes a truncated direct repeat sequence.

In some embodiments, a CRISPR associated protein cleaves the pre-crRNA to form processed or mature crRNA.

In some embodiments, a CRISPR associated protein forms a complex with the mature crRNA and the spacer sequence targets the complex to a complementary sequence in the target nucleic acid. In some embodiments, an RNA guide comprises a direct repeat sequence and a spacer sequence capable of hybridizing under appropriate conditions to a target nucleic acid.

In some embodiments, the RNA guide comprises a direct repeat (DR) sequence of between about 16 and 26 nucleotides long. In some embodiments, the crRNA comprises a nucleotide guide sequence and a DR sequence. The nucleotide guide sequence can be between about 18 and 24 nucleotides long.

In some embodiments, the crRNA sequences can be modified to “dead crRNAs,” “dead guides,” or “dead guide sequences” that can form a complex with a CRISPR-associated protein and bind specific targets without any substantial nuclease activity.

In some embodiments, the crRNA may be chemically modified in the sugar phosphate backbone or base. In some embodiments, the crRNA maybe modified using 2′O-methyl, 2′-F, phosphorothioate or locked nucleic acids to improve nuclease resistance or base pairing. In some embodiments, the crRNA may contain modified bases such as 2-thiouridiene or N6-methyladenosine.

In some embodiments, the crRNA is conjugated with other oligonucleotides, peptides, proteins, tags, dyes, or polyethylene glycol.

In some embodiments, the crRNA may include aptamer or riboswitch sequences that can bind specific target molecules due to their three-dimensional structure.

In some embodiments, a trans-activating RNA (tracrRNA) is associated with crRNA to facilitate formation of a complex with Cas protein. In some embodiments, the tracrRNA sequence is about or more than about 5, 6, 7, 8, 9, 10, 11, 12, 13, 14, 15, 16, 17, 18, 19, 20, 25, 30, 40, 50, 60, 70, 80, 90, 100 or more nucleotides in length. In some embodiments, the tracrRNA is about 70 nucleotides in length.

In some embodiments, the tracrRNA and crRNA are contained in a single transcript called single guide RNA (sgRNA). In some embodiments, the sgRNA includes a loop between the tracrRNA and sgRNA.

In some embodiments, the loop forming sequences are 3, 4, 5 or more nucleotides in length. In some embodiments, the loop has the sequence GAAA, AAAG, CAAA and/or AAAC.

In some embodiments, the tracrRNA and crRNA form a hairpin loop. In some embodiments, sgRNA has at least two or more hairpins. In some embodiments, sgRNA has two, three, four or five hairpins.

In some embodiments, sgRNA includes a transcription termination sequence, which includes a polyT sequences comprising six nucleotides. In some embodiments, the sgRNA comprises a tracrRNA that has one or more point mutations to break a 6×T stretch which acts as a U6 termination signal. For example, in some embodiments, the sgRNA comprises a tracrRNA that has one point mutation. In some embodiments, the sgRNA comprises a tracr RNA that has two point mutations. In some embodiments, the sgRNA comprises a tracrRNA that has three point mutations. In some embodiments, the sgRNA comprises a tracrRNA that has four point mutations. In some embodiments, the sgRNA comprises a tracrRNA that has five point mutations. In some embodiments, the sgRNA comprises a tracrRNA that has five point mutations.

In some embodiments, the sgRNA comprises 6 U (6×U) in the tracrRNA which will act as a U6 termination sequence.

In some embodiments, the tracrRNA is a separate transcript, not contained with crRNA sequence in the same transcript.

### Chemically Modified Guide RNA

In some embodiments, the first end of the guide RNA and/or the second end of the guide RNA comprises a chemical modification to its backbone or to one or more of its bases. For example, chemically modified RNA can comprise chemical synthesis can be used to install highly modified monomers including modified sugars, bases, backbones or functional groups that do not resemble natural nucleotides.

Accordingly, in some embodiments, the first end of the guide RNA and/or the second end of the guide RNA comprises a modified base. In some embodiments, the modified RNA include one or more of the following 2′-O-methoxy-ethyl bases (2′-MOE) such as 2-MethoxyEthoxy A, 2-MethoxyEthoxy MeC, 2-MethoxyEthoxy G, 2-MethoxyEthoxy T. Other modified bases include for example, 2′-O-Methyl RNA bases, and fluoro bases. Various fluoro bases are known, and include for example, Fluoro C, Fluoro U, Fluoro A, Fluoro G bases. Various 2′-O-Methyl modifications can also be used with the methods described herein. For example, the following RNA comprising one or more of the following 2′OMethyl modifications can be used with the methods described: 2′-OMe-5-Methyl-rC, 2′-OMe-rT, 2′-OMe-rI, 2′-OMe-2-Amino-rA, Aminolinker-C6-rC, Aminolinker-C6-rU, 2′-OMe-5-Br-rU, 2′-OMe-5-I-rU, 2-OMe-7-Deaza-rG.

In some embodiments, the first end of the guide RNA and/or second end of the guide RNA comprises one or more of the following modifications: phosphorothioates, 2′O-methyl, 2′ fluoro (2′F), DNA.

In some embodiments, the first end of the guide RNA and/or the second end of the guide RNA comprises 2′OMe modifications at the 3′ and 5′-ends. In some embodiments, the modifications are denoted as mA*, mC*, mG* for modified adenine, modified cytosine and modified guanine, respectively.

In some embodiments, the first end of the guide RNA and/or second end of the guide RNA comprises one or more of the following modifications: 2′-O-2-Methoxyethyl (MOE), locked nucleic acids, bridged nucleic acids, unlocked nucleic acids, peptide nucleic acids, morpholino nucleic acids.

In some embodiments, the first end of the guide RNA and/or second end of the guide RNA comprises one or more of the following base modifications: 2,6-diaminopurine, 2-aminopurine, pseudouracil, N1-methyl-psuedouracil, 5′ methyl cytosine, 2′pyrimidinone (zebularine), thymine.

Other modified bases include for example, 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, Inverted Dideoxy-T, 5-Methyl dC, 5-Methyl dC, 5-Nitroindole, Super TR, 2′-F-r (C,U), 2′—NH2-r (C,U), 2,2′-Anhydro-U, 3′-Desoxy-r (A,C,G,U), 3′-O-Methyl-r (A,C,G,U), rT, rI, 5-Methyl-rC, 2-Amino-rA, rSpacer (Abasic), 7-Deaza-rG, 7-Deaza-rA, 8-Oxo-rG, 5-Halogenated-rU, N-Alkylated-rN.

Other chemically modified RNA can be used herein. For example, the first end of the guide RNA and/or second end of the guide RNA can comprise a modified base such as, for example, 5′, Int, 3′ Azide (NHS Ester); 5′ Hexynyl; 5′, Int, 3′ 5-Octadiynyl dU; 5′, Int Biotin (Azide); 5′, Int 6-FAM (Azide); and 5′, Int 5-TAMRA (Azide). Other examples of RNA nucleotide modifications that can be used with the methods described herein include for example phosphorylation modifications, such as 5′-phosphorylation and 3′-phosphorylation. The RNA can also have one or more of the following modifications: an amino modification, biotinylation, thiol modification, alkyne modifier, adenylation, Azide (NHS Ester), Cholesterol-TEG, and Digoxigenin (NHS Ester).

### CRISPR-Cas

Clustered regularly interspaced short palindromic repeats (CRISPR) was first discovered as an adaptive immune system in bacteria and archaea, and then engineered to generate targeted DNA breaks in living cells and organisms. During the cellular DNA repair process, various DNA changes can be introduced. The diverse and expanding CRISPR toolbox allows programmable genome editing, epigenome editing and transcriptome regulation. Provided herein are methods and compositions comprising CRISPR-Cas in RNA editing.

CRISPR-Cas systems comprise three main types (I, II, and III) based on their Cas gene organization, and the sequence and structure of component proteins. Each of the three CRISPR systems is characterized by a unique Cas gene: Cas3, a target-degrading nuclease/helicase in Type I; Cas9, an RNA-binding and target-degrading nuclease in type II; Cas10, a large protein for multiple functions in type III. The three CRISPR types also differ in their associated effector complexes. Type I Cas systems associate with Cascade effector complexes, type II effector complexes consist of a single Cas9 and one or more RNA molecules, and type III interference complexes are further divided into type III-A (Csm complex targeting DNA) and type III-B (Cmr complex targeting RNA). Cas proteins are important components of effector complexes in all CRISPR-Cas systems.

Genome editing technologies have focused on Class II CRISPR-Cas systems, which contain single-protein effector nucleases for nucleic acid cleavage, specifically, Cas13, a dual-RNA-guided nuclease which requires both CRISPR RNA (crRNA) and tracrRNA and contains both HNH and RuvC nuclease domains, and Cas12a, a single-RNA-guided nuclease which only requires crRNA and contains a single RuvC domain.

While most utilized systems historically targeted DNA, Cas proteins also target RNA by specifically recognizing a given RNA sequence. For example, Type VI (Cas13), Type III (Csm/Cmr), and Type II (Cas9).

The type VI CRISPR-Cas systems require a Cas13 protein and crRNA molecule for activity. There are four subtypes of Type VI Cas comprising Cas13 of differing size and sequence, but all comprising two HEPN domains: VI-A (that uses Cas13a variant, C2c2), VI-B (Cas13b/C2c6), VI-C(Cas13c/C2c7), and VI-D (Cas13d). The HEPN domains are responsible for RNA-targeted nucleolytic activity and are usually located close to terminal ends of the Cas13 protein. The Cas13-cRNA complex binds to targeted ssRNA, triggering a conformational change that brings the two HEPN domains closer to generate a catalytic site, cleaving target RNA. In some embodiments, the HEPN domain is mutated. In some embodiments, Cas is fused to a base editor to carry out deamination.

### Nucleobase Editors

Disclosed herein, are nucleobase editors for editing, modifying or altering a target nucleotide sequence of RNA. Described herein is a nucleobase editor or a base editor comprising a polynucleotide programmable nucleotide binding domain (e.g., PspCas13 or RanCas13) and a nucleobase editing domain (e.g., adenosine deaminase or cytidine deaminase). A polynucleotide programmable nucleotide binding domain (e.g., PspCas13 or RanCas13), when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises RNA. In some embodiments, the target polynucleotide sequence comprises single-stranded RNA. In some embodiments, the target polynucleotide sequence comprises an RNA duplex. In some embodiments, the target polynucleotide sequence comprises a DNA-RNA hybrid. In some embodiments, the target polynucleotide sequence comprises a circular RNA. In some embodiments, the target polynucleotide sequence comprises a messenger RNA. As most of the known genetic variations associated with human disease are point mutations, methods that can more efficiently and cleanly make precise point mutations are needed. RNA base editing systems as provided herein provide a new way to treat genetic and non-genetic diseases in an efficient, reversible and tunable way, with minimal off-target effects.

The base editors provided herein are capable of modifying a specific nucleotide base without generating a significant proportion of indels. The term “indel(s)”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g., mutate or deaminate) a specific nucleotide within a nucleic acid, without generating a large number of insertions or deletions (i.e., indels) in the target nucleotide sequence. In certain embodiments, any of the base editors provided herein are capable of generating a greater proportion of intended modifications (e.g., point mutations or deaminations) versus indels.

In some embodiments, any of base editor systems provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

Some aspects of the disclosure are based on the recognition that any of the base editors provided herein are capable of efficiently generating an intended mutation, such as a point mutation, in a nucleic acid (e.g., RNA) without generating a significant number of unintended mutations, such as unintended point mutations. In some embodiments, any of the base editors provided herein are capable of generating at least 0.01% of intended mutations (i.e. at least 0.01% base editing efficiency). In some embodiments, any of the base editors provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 10:1, at least 11:1, at least 12:1, at least 13:1, at least 14:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more.

The number of intended mutations and indels can be determined using any suitable method, for example, as described in International PCT Application Nos. PCT/2017/045381 (WO2018/027078) and PCT/US2016/058344 (WO2017/070632); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins, or methods of using the fusion proteins provided herein.

### RNA Base Editors

Adenosine Deaminases that Act on RNA (ADARs)

Described herein is an ADAR REPAIR editor that catalyzes A to I editing, and an ADAR RESCUE editor evolved to perform C to U in addition to A to I editing.

ADARs are enzymes that catalyze the conversion of adenosine (A) to inosine (I). ADARs act on different types of RNA, including mRNA. Mutating A to I in mRNA changes the information coded in the mRNA. Inosine preferentially base pairs with cytidine, and is read as guanosine (G) by the translational and splicing machinery. At non-synonymous positions within open reading frames of mRNA, the codon is translated to a different amino acid, which potentially changes protein sequence and function.

Three genes encoding ADAR-like proteins have been reported in vertebrates, (ADAR1, ADAR2 and ADAR3); however, only ADAR1 and ADAR2 are catalytically active.

Structurally, ADARs contain one or more double stranded RNA Binding Domains (dsRBD), and a catalytic deaminase domain (FIG. 2A). The dsRBDs target ADARs to adenosines in RNA, recognizing complicated higher order structures within RNAs. They can bind to perfect duplex RNA, though they will also bind to imperfect structures with bulges, hairpins and mismatches. However, there are no specific motifs based on sequence or structure that are useful to specifically target ADARs to a particular site on RNA. ADAR uses a base flipping mechanism to move the adenosine out of the A-form RNA helix, and into the deaminase catalytic pocket, in order for the A to I conversion to occur. FIG. 2B shows a deamination reaction of adenine to inosine catalyzed by ADAR. A REPAIR base editor which carries out A-to-I editing comprises a dPspCas13b under the control of a T7 promoter fused to a deaminase domain via a linker, and a nuclear export signal. A RESCUE base editor, which can carries out A-to-I and C-to-U editing comprises a dRanCas13b under the control of a T7 promoter fused to an evolved ADAR comprising cytosine deaminase domain via a linker, and a nuclear export signal. The linker comprises a sequence GSGGGGS (SEQ ID NO: 36). FIG. 2C shows a deamination reaction of cytosine to uracil catalyzed by evolved ADAR.

In some embodiments, ADAR is endogenous. ADAR is naturally expressed in a variety of tissues, including cardiomyocytes. Cardiomyocytes depend on the sarcoendoplasmatic reticulum (SER) for normal cellular homeostasis and contractility. Stress stimuli such as oxidative stress, hypoxia, and ischemic insult induce ER stress leading to heart failure. Endogenous ADAR1 functions to counterbalance cardiomyocyte apoptosis. In some embodiments, administration of a guide RNA and Cas protein, recruits endogenous ADAR to the target mRNA site. In some embodiments, the target mRNA is YAP1 or TAZ mRNA in cardiac tissue.

The catalytic domains of Cas13b are mutated to produce an inactive, or “dead” Cas13 (dCas13b) that lacks nucleic acid cleavage activity. In some embodiments, the one or more mutations are in the PAM Interacting, HNH, and or the RuvC domains. In some embodiments, Cas13b is mutated to reduce cleavage activity to less than about 25%, 15%, 10%, 5%, 1%, 0.1%, 0.01% or lower with respect to its non-mutated form. In some embodiments, when coexpressed with a guide RNA, dead Cas13 used to specifically target effector proteins of various functions to specific nucleic acid target sites. In some embodiments, the engineered non-naturally occurring Cas13 is codon-optimized for human cells.

In some embodiments, the base editor is a Prevotella sp. Psp dCas13b-hADAR2 Deaminase Domain REPAIR base editor that catalyzes A to I editing. In some embodiments, the base editor has at least 80% identity to SEQ ID NO: 1. (e.g., 80%, 85%, 90%, 91%, 92%, 93%, 94%, 95%, 96%, 97%, 98%, 99% or more) identical to SEQ ID NO: 1. In some embodiments, the base editor has at least 100% identity to SEQ ID NO: 1.

In some embodiments, the base editor is a Riemerella anatipestifer Ran dCas13b-hADAR2 Evolved RESCUE base editor (evolved to perform C to U in addition to A to I editing). In some embodiments, the base editor has at least 80% identity to SEQ ID NO: 2. (e.g., 80%, 85%, 90%, 91%, 92%, 93%, 94%, 95%, 96%, 97%, 98%, 99% or more) identical to SEQ ID NO: 2. In some embodiments, the base editor has at least 100% identity to SEQ ID NO: 2.

In some embodiments, the base editor is a Prevotella sp. Psp dCas13b-hADAR2 Evolved RESCUE Base Editor (evolved to perform C to U in addition to A to I editing). In some embodiments, the base editor has at least 80% identity to SEQ ID NO: 3. (e.g., 80%, 85%, 90%, 91%, 92%, 93%, 94%, 95%, 96%, 97%, 98%, 99% or more) identical to SEQ ID NO: 3. In some embodiments, the base editor has at least 100% identity to SEQ ID NO: 3. In some embodiments, base editor nucleic acid sequences are provided in Table 4 below.

TABLE 4
Exemplary Cas13b Base Editors

Psp dCas13b-hADAR2 Deaminase Domain REPAIR Base Editor (A to I editing only)
ATGAATATCCCCGCCCTGGTGGAAAACCAGAAGAAATACTTTGGAACTTACTC
CGTGATGGCTATGCTGAATGCTCAGACCGTGCTGGATCACATCCAGAAGGTGG
CCGACATCGAGGGCGAGCAGAACGAGAACAATGAGAATCTGTGGTTCCACCCC
GTGATGAGCCACCTGTACAACGCCAAGAATGGCTATGATAAGCAGCCTGAAAA
GACCATGTTTATCATCGAGCGCCTGCAGTCCTATTTCCCATTTCTGAAGATCAT
GGCCGAGAATCAGAGAGAGTACTCTAACGGCAAGTATAAGCAGAATAGGGTG
GAGGTGAACAGCAATGACATCTTCGAGGTGCTGAAGCGCGCCTTTGGCGTGCT
GAAGATGTACCGGGATCTGACCAACGCCTACAAGACCTACGAGGAGAAGCTG
AATGACGGCTGCGAGTTCCTGACCTCCACAGAGCAGCCACTGTCTGGCATGAT
CAACAATTACTATACCGTGGCCCTGAGAAACATGAATGAGAGGTACGGCTACA
AGACAGAGGATCTGGCCTTTATCCAGGACAAGCGGTTCAAGTTTGTGAAGGAC
GCCTACGGCAAGAAGAAGAGCCAGGTGAACACCGGCTTCTTTCTGTCTCTGCA
GGATTACAATGGCGACACACAGAAGAAGCTGCACCTGAGCGGAGTGGGAATC
GCCCTGCTGATCTGCCTGTTCCTGGATAAGCAGTATATCAACATCTTCCTGAGC
CGCCTGCCCATCTTTAGCTCCTACAATGCCCAGTCCGAGGAGCGGAGAATCAT
CATCCGGAGCTTCGGCATCAACTCCATCAAGCTGCCAAAGGACAGAATCCACT
CTGAGAAGAGCAACAAGTCCGTGGCTATGGACATGCTGAATGAGGTGAAGCG
GTGTCCCGACGAGCTGTTTACCACACTGTCTGCCGAGAAGCAGAGCCGGTTCA
GAATCATCTCCGACGATCACAACGAGGTGCTGATGAAGCGGAGCAGCGACCG
GTTCGTGCCTCTGCTGCTGCAGTACATCGATTATGGCAAGCTGTTCGACCACAT
CAGATTTCACGTGAACATGGGCAAGCTGAGATACCTGCTGAAGGCCGATAAGA
CCTGCATCGACGGCCAGACAAGGGTGCGCGTGATCGAGCAGCCACTGAATGGC
TTCGGCAGACTGGAGGAGGCCGAGACAATGAGGAAGCAGGAGAACGGCACAT
TTGGCAATAGCGGCATCCGGATCAGAGATTTCGAGAACATGAAGAGAGACGAT
GCCAACCCTGCCAATTACCCATATATCGTGGACACCTACACACACTATATCCTG
GAGAACAATAAGGTGGAGATGTTCATCAATGATAAGGAGGACTCCGCCCCACT
GCTGCCTGTGATCGAGGACGATCGCTATGTGGTGAAGACCATCCCCAGCTGTC
GGATGTCCACACTGGAGATCCCTGCTATGGCCTTTCACATGTTCCTGTTTGGCA
GCAAGAAGACCGAGAAGCTGATCGTGGACGTGCACAACCGGTACAAGAGACT
GTTTCAGGCCATGCAGAAGGAGGAGGTGACAGCCGAGAATATCGCCAGCTTCG
GCATCGCCGAGTCCGATCTGCCTCAGAAGATCCTGGACCTGATCTCCGGCAAC
GCCCACGGCAAGGATGTGGACGCCTTTATCAGACTGACCGTGGACGATATGCT
GACCGATACAGAGAGGCGCATCAAGAGGTTCAAGGACGATAGAAAGTCTATC
AGGAGCGCCGACAATAAGATGGGCAAGCGCGGCTTTAAGCAGATCAGCACCG
GCAAGCTGGCCGATTTCCTGGCCAAGGACATCGTGCTGTTTCAGCCCAGCGTG
AACGATGGCGAGAATAAGATCACAGGCCTGAACTACCGGATCATGCAGTCCGC
CATCGCCGTGTACGACTCTGGCGACGATTATGAGGCCAAGCAGCAGTTCAAGC
TGATGTTTGAGAAGGCCAGACTGATCGGCAAGGGCACCACAGAGCCTCACCCA
TTCCTGTATAAGGTGTTTGCCAGGTCCATCCCTGCCAACGCCGTGGAGTTTTAC
GAGAGATACCTGATCGAGCGGAAGTTCTACCTGACCGGCCTGTCTAACGAGAT
CAAGAAGGGCAATAGAGTGGATGTGCCCTTCATCCGGCGGGACCAGAATAAGT
GGAAGACCCCTGCCATGAAGACACTGGGCAGAATCTACAGCGAGGATCTGCCC
GTGGAGCTGCCTAGGCAGATGTTCGACAACGAGATCAAGTCTCACCTGAAGAG
CCTGCCCCAGATGGAGGGCATCGACTTTAACAATGCCAATGTGACCTACCTGA
TCGCCGAGTATATGAAGAGGGTGCTGGACGATGACTTCCAGACCTTCTACCAG
TGGAACAGGAATTACCGCTATATGGATATGCTGAAGGGCGAGTACGACCGCAA
GGGCAGCCTGCAGCACTGCTTCACCTCCGTGGAGGAGAGGGAGGGCCTGTGGA
AGGAGAGGGCCTCCAGAACAGAGCGGTACAGAAAGCAGGCCTCCAACAAGAT
CAGGTCTAATCGCCAGATGCGGAACGCCTCCTCTGAGGAGATCGAGACAATCC
TGGATAAGAGACTGAGCAACTCCAGGAATGAGTACCAGAAGAGCGAGAAAGT
GATCAGGCGCTATAGAGTGCAGGACGCCCTGCTGTTCCTGCTGGCCAAGAAGA
CCCTGACAGAGCTGGCCGATTTCGACGGCGAGAGGTTTAAGCTGAAGGAGATC
ATGCCAGACGCCGAGAAGGGCATCCTGTCTGAGATCATGCCCATGAGCTTCAC
CTTTGAGAAGGGCGGCAAGAAGTATACCATCACAAGCGAGGGCATGAAGCTG
AAGAACTACGGCGATTTCTTTGTGCTGGCCTCCGACAAGAGAATCGGCAATCT
GCTGGAGCTGGTGGGCTCCGACATCGTGAGCAAGGAGGACATCATGGAGGAG
TTCAACAAGTATGATCAGTGTAGGCCCGAGATCAGCTCCATCGTGTTCAATCTG
GAGAAGTGGGCCTTTGACACCTACCCTGAGCTGTCCGCCAGAGTGGATAGGGA
GGAGAAGGTGGACTTTAAGTCTATCCTGAAGATCCTGCTGAACAATAAGAACA
TCAATAAGGAGCAGTCCGACATCCTGCGCAAGATCCGGAACGCCTTCGATGCC
AACAATTATCCAGACAAGGGCGTGGTGGAGATCAAGGCCCTGCCCGAGATCGC
CATGTCTATCAAGAAGGCCTTTGGCGAGTACGCCATCATGAAGGGCAGCCTGC
AGCTGCCACCTCTGGAGCGCCTGACCCTGGGAAGCGGAGGAGGAGGCTCCCAG
CTGCACCTGCCTCAGGTGCTGGCCGACGCCGTGTCTCGGCTGGTGCTGGGCAA
GTTCGGCGATCTGACCGACAACTTTTCTAGCCCACACGCAAGGAGAAAGGTGC
TGGCCGGAGTGGTCATGACCACAGGCACAGATGTGAAGGACGCCAAAGTGAT
CAGCGTGAGCACCGGCACCAAGTGCATCAACGGCGAGTACATGAGCGATAGA
GGCCTGGCCCTGAATGACTGTCACGCCGAGATCATCTCCAGGCGCTCTCTGCTG
CGCTTCCTGTACACCCAGCTGGAGCTGTATCTGAACAATAAGGATGACCAGAA
GCGGAGCATCTTCCAGAAGTCCGAGCGCGGCGGCTTTCGGCTGAAGGAGAACG
TGCAGTTCCACCTGTACATCTCCACATCTCCATGCGGCGATGCCAGAATCTTTT
CTCCACACGAGCCAATCCTGGAGGAGCCTGCAGACCGCCACCCAAACAGAAA
GGCCAGGGGCCAGCTGCGGACCAAGATCGAGAGCGGCCAAGGCACAATCCCC
GTGAGGAGCAATGCCTCCATCCAGACCTGGGATGGCGTGCTGCAGGGAGAGCG
CCTGCTGACAATGAGCTGTTCCGACAAGATCGCAAGGTGGAACGTGGTGGGCA
TCCAGGGCTCTCTGCTGAGCATCTTCGTGGAGCCCATCTACTTTTCCTCTATCAT
CCTGGGCTCTCTGTATCACGGCGATCACCTGAGCCGCGCCATGTACCAGCGGA
TCTCCAATATCGAGGACCTGCCACCCCTGTATACCCTGAACAAGCCTCTGCTGT
CCGGCATCTCTAATGCAGAGGCAAGGCAGCCAGGCAAGGCACCAAACTTCTCT
GTGAATTGGACAGTGGGCGATAGCGCCATCGAAGTGATCAACGCCACCACAGG
CAAGGACGAGCTGGGAAGGGCCTCCCGGCTGTGCAAGCACGCCCTGTACTGTA
GATGGATGAGGGTGCACGGCAAGGTGCCTTCTCACCTGCTGCGCAGCAAGATC
ACCAAGCCAAACGTGTACCACGAGAGCAAGCTGGCCGCAAAGGAGTACCAGG
CAGCAAAGGCAAGGCTGTTCACAGCCTTTATCAAGGCAGGGCTGGGGGCTTGG
GTGGAAAAACCTACCGAGCAGGACCAGTTCTCACTGACATAA (SEQ ID NO: 1)
Ran dCas13b-hADAR2 Evolved RESCUE Base Editor (Evolved to perform C to U in
addition to A to I editing)
ATGGAGAAGCCCCTGCTGCCTAACGTGTATACCCTGAAGCACAAGTTCTTTTGG
GGCGCCTTCCTGAATATCGCCCGGCACAACGCCTTTATCACCATCTGCCACATC
AATGAGCAGCTGGGCCTGAAAACCCCCAGCAACGACGATAAGATCGTGGACG
TGGTGTGCGAGACATGGAACAATATCCTGAACAATGACCACGATCTGCTGAAG
AAGTCCCAGCTGACCGAGCTGATCCTGAAGCACTTCCCTTTTCTGACAGCCATG
TGCTACCACCCCCCTAAGAAGGAGGGCAAGAAGAAGGGCCACCAGAAGGAGC
AGCAGAAGGAGAAGGAGAGCGAGGCACAGTCCCAGGCAGAGGCCCTGAACCC
ATCCAAGCTGATCGAGGCCCTGGAGATCCTGGTGAATCAGCTGCACTCTCTGG
CAAACTACTATAGCGCCTATAAGCACAAGAAGCCCGACGCCGAGAAGGATATC
TTCAAGCACCTGTATAAGGCCTTCGACGCCTCTCTGAGAATGGTGAAGGAGGA
TTATAAGGCCCACTTCACCGTGAATCTGACAAGGGACTTTGCCCACCTGAACC
GCAAGGGCAAGAACAAGCAGGACAATCCCGACTTCAACAGATACAGATTCGA
GAAGGATGGCTTCTTTACCGAGTCTGGCCTGCTGTTCTTTACAAATCTGTTTCT
GGACAAGAGGGATGCCTATTGGATGCTGAAGAAGGTGTCCGGCTTCAAGGCCT
CTCACAAGCAGCGCGAGAAGATGACCACAGAGGTGTTTTGCAGGAGCCGCATC
CTGCTGCCCAAGCTGAGGCTGGAGTCCCGCTACGACCACAACCAGATGCTGCT
GGATATGCTGTCTGAGCTGAGCAGATGTCCTAAGCTGCTGTATGAGAAGCTGA
GCGAGGAGAATAAGAAGCACTTCCAGGTGGAGGCCGACGGCTTTCTGGATGA
GATCGAGGAGGAGCAGAACCCATTCAAGGACACCCTGATCCGGCACCAGGAT
AGATTCCCCTACTTTGCCCTGAGGTATCTGGACCTGAATGAGTCCTTCAAGTCT
ATCCGCTTTCAGGTGGATCTGGGCACATACCACTATTGTATCTACGACAAGAA
GATCGGCGATGAGCAGGAGAAGAGGCACCTGACCCGCACACTGCTGTCCTTCG
GCCGGCTGCAGGACTTTACCGAGATCAACAGACCCCAGGAGTGGAAGGCCCTG
ACCAAGGACCTGGATTACAAGGAGACATCCAATCAGCCTTTCATCTCTAAGAC
CACACCACACTATCACATCACCGACAACAAGATCGGCTTTCGGCTGGGCACAA
GCAAGGAGCTGTACCCCTCCCTGGAGATCAAGGATGGCGCCAATAGAATCGCC
AAGTACCCTTATAACTCCGGCTTCGTGGCCCACGCCTTTATCTCTGTGCACGAG
CTGCTGCCTCTGATGTTCTACCAGCACCTGACCGGCAAGAGCGAGGACCTGCT
GAAGGAGACAGTGCGGCACATCCAGAGAATCTATAAGGACTTCGAGGAGGAG
CGGATCAATACCATCGAGGATCTGGAGAAGGCAAACCAGGGCAGACTGCCAC
TGGGAGCCTTTCCCAAGCAGATGCTGGGCCTGCTGCAGAATAAGCAGCCTGAT
CTGTCCGAGAAGGCCAAGATCAAGATCGAGAAGCTGATCGCCGAGACAAAGC
TGCTGTCTCACAGGCTGAACACAAAGCTGAAGAGCTCCCCAAAGCTGGGCAAG
CGGAGAGAGAAGCTGATCAAGACAGGCGTGCTGGCCGACTGGCTGGTGAAGG
ATTTCATGCGCTTTCAGCCCGTGGCCTACGACGCCCAGAATCAGCCTATCAAGT
CTAGCAAGGCCAACTCCACCGAGTTCTGGTTTATCAGGCGCGCCCTGGCCCTGT
ACGGAGGAGAGAAGAATAGGCTGGAGGGCTATTTCAAGCAGACCAACCTGAT
CGGCAACACAAATCCACACCCCTTCCTGAACAAGTTTAATTGGAAGGCCTGCA
GGAATCTGGTGGATTTCTACCAGCAGTATCTGGAGCAGCGCGAGAAGTTTCTG
GAGGCCATCAAGAACCAGCCATGGGAGCCCTACCAGTATTGCCTGCTGCTGAA
GATCCCTAAGGAGAACAGGAAGAATCTGGTGAAGGGATGGGAGCAGGGAGGA
ATCTCTCTGCCACGGGGCCTGTTTACCGAGGCCATCAGAGAGACACTGTCTGA
GGACCTGATGCTGAGCAAGCCAATCCGCAAGGAGATCAAGAAGCACGGCCGG
GTGGGCTTCATCAGCAGAGCCATCACCCTGTACTTTAAGGAGAAGTATCAGGA
TAAGCACCAGAGCTTCTACAATCTGTCCTATAAGCTGGAGGCAAAGGCACCAC
TGCTGAAGCGGGAGGAGCACTACGAGTATTGGCAGCAGAACAAGCCTCAGTCT
CCAACCGAGAGCCAGAGGCTGGAGCTGCACACAAGCGACCGCTGGAAGGATT
ACCTGCTGTATAAGAGATGGCAGCACCTGGAGAAGAAGCTGCGGCTGTACCGG
AATCAGGACGTGATGCTGTGGCTGATGACCCTGGAGCTGACAAAGAACCACTT
CAAGGAGCTGAACCTGAATTATCACCAGCTGAAGCTGGAGAATCTGGCCGTGA
ACGTGCAGGAGGCCGATGCCAAGCTGAACCCTCTGAATCAGACCCTGCCCATG
GTGCTGCCTGTGAAGGTGTACCCTGCCACAGCCTTTGGCGAGGTGCAGTACCA
CAAGACCCCAATCAGGACAGTGTATATCCGCGAGGAGCACACCAAGGCCCTGA
AGATGGGCAACTTCAAGGCCCTGGTGAAGGACCGGAGACTGAATGGCCTGTTC
TCTTTTATCAAGGAGGAGAACGATACACAGAAGCACCCAATCAGCCAGCTGCG
GCTGAGGCGCGAGCTGGAGATCTACCAGTCTCTGAGAGTGGACGCCTTCAAGG
AGACACTGAGCCTGGAGGAGAAGCTGCTGAATAAGCACACATCCCTGTCCTCT
CTGGAGAACGAGTTTAGGGCCCTGCTGGAGGAGTGGAAGAAGGAGTATGCCG
CCAGCTCCATGGTGACAGACGAGCACATCGCCTTCATCGCCAGCGTGGCCAAT
GCCTTCTGCGCCAACCAGTACCCCTTCTACAAGGAGGCCCTGCACGCCCCTATC
CCACTGTTCACCGTGGCCCAGCCCACCACAGAGGAGAAGGATGGCCTGGGAAT
CGCAGAGGCCCTGCTGAAGGTGCTGAGGGAGTACTGTGAGATCGTGAAGTCCC
AGATCGGATCCAGCCTCCAAAAAAAGTTGGAGGAATTAGAACTAGGATCCCAG
CTGCACCTGCCTCAGGTGCTGGCCGACGCCGTGTCTCGGCTGGTGATCGGCAA
GTTCGGCGATCTGACCGACAACTTTTCTAGCCCACACGCAAGGAGAATCGGCC
TGGCCGGAGTGGTCATGACCACAGGCACAGATGTGAAGGACGCCAAAGTGAT
CTGCGTGAGCACCGGCAGCAAGTGCATCAACGGCGAGTACCTGAGCGATAGA
GGCCTGGCCCTGAATGACTGTCACGCCGAGATCGTGTCCAGGCGCTCTCTGCTG
CGCTTCCTGTACACCCAGCTGGAGCTGTATCTGAACAATGAGGATGACCAGAA
GCGGAGCATCTTCCAGAAGTCCGAGCGCGGCGGCTTTCGGCTGAAGGAGAACA
TCCAGTTCCACCTGTACATCTCCACATCTCCATGCGGCGATGCCAGAATCTTTT
CTCCACACGAGGCAATCCTGGAGGAGCCTGCAGACCGCCACCCAAACAGAAA
GGCCAGGGGCCAGCTGCGGACCAAGATCGAGGCCGGCCAAGGCACAATCCCC
GTGAGGAACAATGCCTCCATCCAGACCTGGGATGGCGTGCTGCAGGGAGAGCG
CCTGCTGACAATGAGCTGTTCCGACAAGATCGCAAGGTGGAACGTGGTGGGCA
TCCAGGGCTCTCTGCTGAGCATCTTCGTGGAGCCCATCTACTTTTCCTCTATCAT
CCTGGGCTCTCTGTATCACGGCGATCACCTGAGCCGCGCCATGTACCAGCGGA
TCTCCAATATCGAGGACCTGCCACCCCTGTATACCCTGAACAAGCCTCTGCTGA
CAGGCATCTCTAATGCAGAGGCAAGGCAGCCAGGCAAGGCACCAATCTTCTCT
GTGAATTGGACAGTGGGCGATAGCGCCATCGAAGTGATCAACGCCACCACAGG
CAAGGGCGAGCTGGGAAGGGCCTCCCGGCTGTGCAAGCACGCCCTGTACTGTA
GATGGATGAGGGTGCACGGCAAGGTGCCTTCTCACCTGCTGCGCAGCAAGATC
ACCAAGCCAAACGTGTACCACGAGACCAAGCTGGCCGCAAAGGAGTACCAGG
CAGCAAAGGCAAGGCTGTTCACAGCCTTTATCAAGGCAGGGCTGGGGGCTTGG
GTGGAAAAACCTACCGAGCAGGACCAGTTCTCACTGACACCTTAA (SEQ ID NO: 2)
Psp dCas13b-hADAR2 Evolved RESCUE Base Editor (Evolved to perform C to U in
addition to A to I editing)
ATGAATATCCCCGCCCTGGTGGAAAACCAGAAGAAATACTTTGGAACTTACTC
CGTGATGGCTATGCTGAATGCTCAGACCGTGCTGGATCACATCCAGAAGGTGG
CCGACATCGAGGGCGAGCAGAACGAGAACAATGAGAATCTGTGGTTCCACCCC
GTGATGAGCCACCTGTACAACGCCAAGAATGGCTATGATAAGCAGCCTGAAAA
GACCATGTTTATCATCGAGCGCCTGCAGTCCTATTTCCCATTTCTGAAGATCAT
GGCCGAGAATCAGAGAGAGTACTCTAACGGCAAGTATAAGCAGAATAGGGTG
GAGGTGAACAGCAATGACATCTTCGAGGTGCTGAAGCGCGCCTTTGGCGTGCT
GAAGATGTACCGGGATCTGACCAACGCCTACAAGACCTACGAGGAGAAGCTG
AATGACGGCTGCGAGTTCCTGACCTCCACAGAGCAGCCACTGTCTGGCATGAT
CAACAATTACTATACCGTGGCCCTGAGAAACATGAATGAGAGGTACGGCTACA
AGACAGAGGATCTGGCCTTTATCCAGGACAAGCGGTTCAAGTTTGTGAAGGAC
GCCTACGGCAAGAAGAAGAGCCAGGTGAACACCGGCTTCTTTCTGTCTCTGCA
GGATTACAATGGCGACACACAGAAGAAGCTGCACCTGAGCGGAGTGGGAATC
GCCCTGCTGATCTGCCTGTTCCTGGATAAGCAGTATATCAACATCTTCCTGAGC
CGCCTGCCCATCTTTAGCTCCTACAATGCCCAGTCCGAGGAGCGGAGAATCAT
CATCCGGAGCTTCGGCATCAACTCCATCAAGCTGCCAAAGGACAGAATCCACT
CTGAGAAGAGCAACAAGTCCGTGGCTATGGACATGCTGAATGAGGTGAAGCG
GTGTCCCGACGAGCTGTTTACCACACTGTCTGCCGAGAAGCAGAGCCGGTTCA
GAATCATCTCCGACGATCACAACGAGGTGCTGATGAAGCGGAGCAGCGACCG
GTTCGTGCCTCTGCTGCTGCAGTACATCGATTATGGCAAGCTGTTCGACCACAT
CAGATTTCACGTGAACATGGGCAAGCTGAGATACCTGCTGAAGGCCGATAAGA
CCTGCATCGACGGCCAGACAAGGGTGCGCGTGATCGAGCAGCCACTGAATGGC
TTCGGCAGACTGGAGGAGGCCGAGACAATGAGGAAGCAGGAGAACGGCACAT
TTGGCAATAGCGGCATCCGGATCAGAGATTTCGAGAACATGAAGAGAGACGAT
GCCAACCCTGCCAATTACCCATATATCGTGGACACCTACACACACTATATCCTG
GAGAACAATAAGGTGGAGATGTTCATCAATGATAAGGAGGACTCCGCCCCACT
GCTGCCTGTGATCGAGGACGATCGCTATGTGGTGAAGACCATCCCCAGCTGTC
GGATGTCCACACTGGAGATCCCTGCTATGGCCTTTCACATGTTCCTGTTTGGCA
GCAAGAAGACCGAGAAGCTGATCGTGGACGTGCACAACCGGTACAAGAGACT
GTTTCAGGCCATGCAGAAGGAGGAGGTGACAGCCGAGAATATCGCCAGCTTCG
GCATCGCCGAGTCCGATCTGCCTCAGAAGATCCTGGACCTGATCTCCGGCAAC
GCCCACGGCAAGGATGTGGACGCCTTTATCAGACTGACCGTGGACGATATGCT
GACCGATACAGAGAGGCGCATCAAGAGGTTCAAGGACGATAGAAAGTCTATC
AGGAGCGCCGACAATAAGATGGGCAAGCGCGGCTTTAAGCAGATCAGCACCG
GCAAGCTGGCCGATTTCCTGGCCAAGGACATCGTGCTGTTTCAGCCCAGCGTG
AACGATGGCGAGAATAAGATCACAGGCCTGAACTACCGGATCATGCAGTCCGC
CATCGCCGTGTACGACTCTGGCGACGATTATGAGGCCAAGCAGCAGTTCAAGC
TGATGTTTGAGAAGGCCAGACTGATCGGCAAGGGCACCACAGAGCCTCACCCA
TTCCTGTATAAGGTGTTTGCCAGGTCCATCCCTGCCAACGCCGTGGAGTTTTAC
GAGAGATACCTGATCGAGCGGAAGTTCTACCTGACCGGCCTGTCTAACGAGAT
CAAGAAGGGCAATAGAGTGGATGTGCCCTTCATCCGGCGGGACCAGAATAAGT
GGAAGACCCCTGCCATGAAGACACTGGGCAGAATCTACAGCGAGGATCTGCCC
GTGGAGCTGCCTAGGCAGATGTTCGACAACGAGATCAAGTCTCACCTGAAGAG
CCTGCCCCAGATGGAGGGCATCGACTTTAACAATGCCAATGTGACCTACCTGA
TCGCCGAGTATATGAAGAGGGTGCTGGACGATGACTTCCAGACCTTCTACCAG
TGGAACAGGAATTACCGCTATATGGATATGCTGAAGGGCGAGTACGACCGCAA
GGGCAGCCTGCAGCACTGCTTCACCTCCGTGGAGGAGAGGGAGGGCCTGTGGA
AGGAGAGGGCCTCCAGAACAGAGCGGTACAGAAAGCAGGCCTCCAACAAGAT
CAGGTCTAATCGCCAGATGCGGAACGCCTCCTCTGAGGAGATCGAGACAATCC
TGGATAAGAGACTGAGCAACTCCAGGAATGAGTACCAGAAGAGCGAGAAAGT
GATCAGGCGCTATAGAGTGCAGGACGCCCTGCTGTTCCTGCTGGCCAAGAAGA
CCCTGACAGAGCTGGCCGATTTCGACGGCGAGAGGTTTAAGCTGAAGGAGATC
ATGCCAGACGCCGAGAAGGGCATCCTGTCTGAGATCATGCCCATGAGCTTCAC
CTTTGAGAAGGGCGGCAAGAAGTATACCATCACAAGCGAGGGCATGAAGCTG
AAGAACTACGGCGATTTCTTTGTGCTGGCCTCCGACAAGAGAATCGGCAATCT
GCTGGAGCTGGTGGGCTCCGACATCGTGAGCAAGGAGGACATCATGGAGGAG
TTCAACAAGTATGATCAGTGTAGGCCCGAGATCAGCTCCATCGTGTTCAATCTG
GAGAAGTGGGCCTTTGACACCTACCCTGAGCTGTCCGCCAGAGTGGATAGGGA
GGAGAAGGTGGACTTTAAGTCTATCCTGAAGATCCTGCTGAACAATAAGAACA
TCAATAAGGAGCAGTCCGACATCCTGCGCAAGATCCGGAACGCCTTCGATGCC
AACAATTATCCAGACAAGGGCGTGGTGGAGATCAAGGCCCTGCCCGAGATCGC
CATGTCTATCAAGAAGGCCTTTGGCGAGTACGCCATCATGAAGGGCAGCCTGC
AGCTGCCACCTCTGGAGCGCCTGACCCTGGGATCCCAGCTGCACCTGCCTCAG
GTGCTGGCCGACGCCGTGTCTCGGCTGGTGATCGGCAAGTTCGGCGATCTGAC
CGACAACTTTTCTAGCCCACACGCAAGGAGAATCGGCCTGGCCGGAGTGGTCA
TGACCACAGGCACAGATGTGAAGGACGCCAAAGTGATCTGCGTGAGCACCGG
CAGCAAGTGCATCAACGGCGAGTACCTGAGCGATAGAGGCCTGGCCCTGAATG
ACTGTCACGCCGAGATCGTGTCCAGGCGCTCTCTGCTGCGCTTCCTGTACACCC
AGCTGGAGCTGTATCTGAACAATGAGGATGACCAGAAGCGGAGCATCTTCCAG
AAGTCCGAGCGCGGCGGCTTTCGGCTGAAGGAGAACATCCAGTTCCACCTGTA
CATCTCCACATCTCCATGCGGCGATGCCAGAATCTTTTCTCCACACGAGGCAAT
CCTGGAGGAGCCTGCAGACCGCCACCCAAACAGAAAGGCCAGGGGCCAGCTG
CGGACCAAGATCGAGGCCGGCCAAGGCACAATCCCCGTGAGGAACAATGCCT
CCATCCAGACCTGGGATGGCGTGCTGCAGGGAGAGCGCCTGCTGACAATGAGC
TGTTCCGACAAGATCGCAAGGTGGAACGTGGTGGGCATCCAGGGCTCTCTGCT
GAGCATCTTCGTGGAGCCCATCTACTTTTCCTCTATCATCCTGGGCTCTCTGTAT
CACGGCGATCACCTGAGCCGCGCCATGTACCAGCGGATCTCCAATATCGAGGA
CCTGCCACCCCTGTATACCCTGAACAAGCCTCTGCTGACAGGCATCTCTAATGC
AGAGGCAAGGCAGCCAGGCAAGGCACCAATCTTCTCTGTGAATTGGACAGTGG
GCGATAGCGCCATCGAAGTGATCAACGCCACCACAGGCAAGGGCGAGCTGGG
AAGGGCCTCCCGGCTGTGCAAGCACGCCCTGTACTGTAGATGGATGAGGGTGC
ACGGCAAGGTGCCTTCTCACCTGCTGCGCAGCAAGATCACCAAGCCAAACGTG
TACCACGAGACCAAGCTGGCCGCAAAGGAGTACCAGGCAGCAAAGGCAAGGC
TGTTCACAGCCTTTATCAAGGCAGGGCTGGGGGCTTGGGTGGAAAAACCTACC
GAGCAGGACCAGTTCTCACTGACACCTTAA (SEQ ID NO: 3)

TABLE 5
Amino Acid Sequence of Exemplary Cas13b Base Editors

dPsp-Cas13b fused REPAIR VI human ADAR2 Deaminase Domain V1 (E488Q) base
editor
MNIPALVENQKKYFGTYSVMAMLNAQTVLDHIQKVADIEGEQNENNENLWFHPV
MSHLYNAKNGYDKQPEKTMFIIERLQSYFPFLKIMAENQREYSNGKYKQNRVEVN
SNDIFEVLKRAFGVLKMYRDLTNAYKTYEEKLNDGCEFLTSTEQPLSGMINNYYT
VALRNMNERYGYKTEDLAFIQDKRFKFVKDAYGKKKSQVNTGFFLSLQDYNGDT
QKKLHLSGVGIALLICLFLDKQYINIFLSRLPIFSSYNAQSEERRIIIRSFGINSIKLPKD
RIHSEKSNKSVAMDMLNEVKRCPDELFTTLSAEKQSRFRIISDDHNEVLMKRSSDR
FVPLLLQYIDYGKLFDHIRFHVNMGKLRYLLKADKTCIDGQTRVRVIEQPLNGFGR
LEEAETMRKQENGTFGNSGIRIRDFENMKRDDANPANYPYIVDTYTHYILENNKV
EMFINDKEDSAPLLPVIEDDRYVVKTIPSCRMSTLEIPAMAFHMFLFGSKKTEKLIV
DVHNRYKRLFQAMQKEEVTAENIASFGIAESDLPQKILDLISGNAHGKDVDAFIRL
TVDDMLTDTERRIKRFKDDRKSIRSADNKMGKRGFKQISTGKLADFLAKDIVLFQP
SVNDGENKITGLNYRIMQSAIAVYDSGDDYEAKQQFKLMFEKARLIGKGTTEPHPF
LYKVFARSIPANAVEFYERYLIERKFYLTGLSNEIKKGNRVDVPFIRRDQNKWKTP
AMKTLGRIYSEDLPVELPRQMFDNEIKSHLKSLPQMEGIDENNANVTYLIAEYMKR
VLDDDFQTFYQWNRNYRYMDMLKGEYDRKGSLQHCFTSVEEREGLWKERASRT
ERYRKQASNKIRSNRQMRNASSEEIETILDKRLSNSRNEYQKSEKVIRRYRVQDAL
LFLLAKKTLTELADFDGERFKLKEIMPDAEKGILSEIMPMSFTFEKGGKKYTITSEG
MKLKNYGDFFVLASDKRIGNLLELVGSDIVSKEDIMEEFNKYDQCRPEISSIVFNLE
KWAFDTYPELSARVDREEKVDFKSILKILLNNKNINKEQSDILRKIRNAFDANNYP
DKGVVEIKALPEIAMSIKKAFGEYAIMKGSLQLPPLERLTLGSGGGGSQLHLPQV
LADAVSRLVLGKFGDLTDNFSSPHARRKVLAGVVMTTGTDVKDAKVISVSTGTKCINGE
YMSDRGLALNDCHAEIISRRSLLRFLYTQLELYLNNKDDQKRSIFQKSERGGFRLKENVQ
FHLYISTSPCGDARIFSPHEPILEEPADRHPNRKARGQLRTKIESGQGTIPVRSNASIQTW
DGVLQGERLLTMSCSDKIARWNVVGIQGSLLSIFVEPIYFSSIILGSLYHGDHLSRAMYQR
ISNIEDLPPLYTLNKPLLSGISNAEARQPGKAPNFSVNWTVGDSAIEVINATTGKDELGRA
SRLCKHALYCRWMRVHGKVPSHLLRSKITKPNVYHESKLAAKEYQAAKARLFTAFIKAG
LGAWVEKPTEQDQFSLT (SEQ ID NO: 64)
Regular: dPsp-Cas13b
Bold: GS-NES-GSLinker
Italics: human ADAR2 Deaminase Domain V1 (E488Q)
dPsp-Cas13b fused to REPAIR V2 human ADAR2 Deaminase Domain (E488Q + T375G)
base editor
MNIPALVENQKKYFGTYSVMAMLNAQTVLDHIQKVADIEGEQNENNENLWFHPV
MSHLYNAKNGYDKQPEKTMFIIERLQSYFPFLKIMAENQREYSNGKYKQNRVEVN
SNDIFEVLKRAFGVLKMYRDLTNAYKTYEEKLNDGCEFLTSTEQPLSGMINNYYT
VALRNMNERYGYKTEDLAFIQDKRFKFVKDAYGKKKSQVNTGFFLSLQDYNGDT
QKKLHLSGVGIALLICLFLDKQYINIFLSRLPIFSSYNAQSEERRIIIRSFGINSIKLPKD
RIHSEKSNKSVAMDMLNEVKRCPDELFTTLSAEKQSRFRIISDDHNEVLMKRSSDR
FVPLLLQYIDYGKLFDHIRFHVNMGKLRYLLKADKTCIDGQTRVRVIEQPLNGFGR
LEEAETMRKQENGTFGNSGIRIRDFENMKRDDANPANYPYIVDTYTHYILENNKV
EMFINDKEDSAPLLPVIEDDRYVVKTIPSCRMSTLEIPAMAFHMFLFGSKKTEKLIV
DVHNRYKRLFQAMQKEEVTAENIASFGIAESDLPQKILDLISGNAHGKDVDAFIRL
TVDDMLTDTERRIKRFKDDRKSIRSADNKMGKRGFKQISTGKLADFLAKDIVLFQP
SVNDGENKITGLNYRIMQSAIAVYDSGDDYEAKQQFKLMFEKARLIGKGTTEPHPF
LYKVFARSIPANAVEFYERYLIERKFYLTGLSNEIKKGNRVDVPFIRRDQNKWKTP
AMKTLGRIYSEDLPVELPRQMFDNEIKSHLKSLPQMEGIDENNANVTYLIAEYMKR
VLDDDFQTFYQWNRNYRYMDMLKGEYDRKGSLQHCFTSVEEREGLWKERASRT
ERYRKQASNKIRSNRQMRNASSEEIETILDKRLSNSRNEYQKSEKVIRRYRVQDAL
LFLLAKKTLTELADFDGERFKLKEIMPDAEKGILSEIMPMSFTFEKGGKKYTITSEG
MKLKNYGDFFVLASDKRIGNLLELVGSDIVSKEDIMEEFNKYDQCRPEISSIVENLE
KWAFDTYPELSARVDREEKVDFKSILKILLNNKNINKEQSDILRKIRNAFDANNYP
DKGVVEIKALPEIAMSIKKAFGEYAIMKGSLQLPPLERLTLGSGGGGSQLHLPQV
LADAVSRLVLGKFGDLTDNFSSPHARRKVLAGVVMTTGTDVKDAKVISVSTGGKCINGE
YMSDRGLALNDCHAEIISRRSLLRFLYTQLELYLNNKDDQKRSIFQKSERGGFRLKENVQ
FHLYISTSPCGDARIFSPHEPILEEPADRHPNRKARGQLRTKIESGQGTIPVRSNASIQTW
DGVLQGERLLTMSCSDKIARWNVVGIQGSLLSIFVEPIYFSSIILGSLYHGDHLSRAMYQR
ISNIEDLPPLYTLNKPLLSGISNAEARQPGKAPNFSVNWTVGDSAIEVINATTGKDELGRA
SRLCKHALYCRWMRVHGKVPSHLLRSKITKPNVYHESKLAAKEYQAAKARLFTAFIKAG
LGAWVEKPTEQDQFSLT (SEQ ID NO: 65)
Regular: dPsp-Cas13b
Bold: GS-NES-GSLinker
Italics: human ADAR2 Deaminase Domain (E488Q + T375G)
Ran-dCas13b fused to RESCUE base editor (human ADAR2 Deaminase Domain evolved
from wt hADAR2 Deaminase Domain)
MEKPLLPNVYTLKHKFFWGAFLNIARHNAFITICHINEQLGLKTPSNDDKIVDVVC
ETWNNILNNDHDLLKKSQLTELILKHFPFLTAMCYHPPKKEGKKKGHQKEQQKEK
ESEAQSQAEALNPSKLIEALEILVNQLHSLANYYSAYKHKKPDAEKDIFKHLYKAF
DASLRMVKEDYKAHFTVNLTRDFAHLNRKGKNKQDNPDFNRYRFEKDGFFTESG
LLFFTNLFLDKRDAYWMLKKVSGFKASHKQREKMTTEVFCRSRILLPKLRLESRY
DHNQMLLDMLSELSRCPKLLYEKLSEENKKHFQVEADGFLDEIEEEQNPFKDTLIR
HQDRFPYFALRYLDLNESFKSIRFQVDLGTYHYCIYDKKIGDEQEKRHLTRTLLSF
GRLQDFTEINRPQEWKALTKDLDYKETSNQPFISKTTPHYHITDNKIGFRLGTSKEL
YPSLEIKDGANRIAKYPYNSGFVAHAFISVHELLPLMFYQHLTGKSEDLLKETVRHI
QRIYKDFEEERINTIEDLEKANQGRLPLGAFPKQMLGLLQNKQPDLSEKAKIKIEKL
IAETKLLSHRLNTKLKSSPKLGKRREKLIKTGVLADWLVKDFMRFQPVAYDAQNQ
PIKSSKANSTEFWFIRRALALYGGEKNRLEGYFKQTNLIGNTNPHPFLNKENWKAC
RNLVDFYQQYLEQREKFLEAIKNQPWEPYQYCLLLKIPKENRKNLVKGWEQGGIS
LPRGLFTEAIRETLSEDLMLSKPIRKEIKKHGRVGFISRAITLYFKEKYQDKHQSFYN
LSYKLEAKAPLLKREEHYEYWQQNKPQSPTESQRLELHTSDRWKDYLLYKRWQH
LEKKLRLYRNQDVMLWLMTLELTKNHFKELNLNYHQLKLENLAVNVQEADAKL
NPLNQTLPMVLPVKVYPATAFGEVQYHKTPIRTVYIREEHTKALKMGNFKALVKD
RRLNGLFSFIKEENDTQKHPISQLRLRRELEIYQSLRVDAFKETLSLEEKLLNKHTSL
SSLENEFRALLEEWKKEYAASSMVTDEHIAFIASVANAFCANQYPFYKEALHAPIP
LFTVAQPTTEEKDGLGIAEALLKVLREYCEIVKSQIGSSLQKKLEELELQLHLPQV
LADAVSRLVIGKFGDLTDNFSSPHARRIGLAGVVMTTGTDVKDAKVICVSTGSKCINGEY
LSDRGLALNDCHAEIVSRRSLLRFLYTQLELYLNNEDDQKRSIFQKSERGGFRLKENIQF
HLYISTSPCGDARIFSPHEAILEEPADRHPNRKARGQLRTKIEAGQGTIPVRNNASIQTW
DGVLQGERLLTMSCSDKIARWNVVGIQGSLLSIFVEPIYFSSIILGSLYHGDHLSRAMYQR
ISNIEDLPPLYTLNKPLLTGISNAEARQPGKAPIFSVNWTVGDSAIEVINATTGKGELGRAS
RLCKHALYCRWMRVHGKVPSHLLRSKITKPNVYHETKLAAKEYQAAKARLFTAFIKAGL
GAWVEKPTEQDQFSLTP (SEQ ID NO: 66)
Underlined: Ran-dCas13b
Bold: GS-Mapk NES
Regular: RESCUE (human ADAR2 Deaminase Domain evolved from wt hADAR2
Deaminase Domain)
dPsp-Cas13b fused to RESCUE base editor (human ADAR2 Deaminase Domain evolved
from wt hADAR2 Deaminase Domain)
MNIPALVENQKKYFGTYSVMAMLNAQTVLDHIQKVADIEGEQNENNENLWFHPV
MSHLYNAKNGYDKQPEKTMFIIERLQSYFPFLKIMAENQREYSNGKYKQNRVEVN
SNDIFEVLKRAFGVLKMYRDLTNAYKTYEEKLNDGCEFLTSTEQPLSGMINNYYT
VALRNMNERYGYKTEDLAFIQDKRFKFVKDAYGKKKSQVNTGFFLSLQDYNGDT
QKKLHLSGVGIALLICLFLDKQYINIFLSRLPIFSSYNAQSEERRIIIRSFGINSIKLPKD
RIHSEKSNKSVAMDMLNEVKRCPDELFTTLSAEKQSRFRIISDDHNEVLMKRSSDR
FVPLLLQYIDYGKLFDHIRFHVNMGKLRYLLKADKTCIDGQTRVRVIEQPLNGFGR
LEEAETMRKQENGTFGNSGIRIRDFENMKRDDANPANYPYIVDTYTHYILENNKV
EMFINDKEDSAPLLPVIEDDRYVVKTIPSCRMSTLEIPAMAFHMFLFGSKKTEKLIV
DVHNRYKRLFQAMQKEEVTAENIASFGIAESDLPQKILDLISGNAHGKDVDAFIRL
TVDDMLTDTERRIKRFKDDRKSIRSADNKMGKRGFKQISTGKLADFLAKDIVLFQP
SVNDGENKITGLNYRIMQSAIAVYDSGDDYEAKQQFKLMFEKARLIGKGTTEPHPF
LYKVFARSIPANAVEFYERYLIERKFYLTGLSNEIKKGNRVDVPFIRRDQNKWKTP
AMKTLGRIYSEDLPVELPRQMFDNEIKSHLKSLPQMEGIDENNANVTYLIAEYMKR
VLDDDFQTFYQWNRNYRYMDMLKGEYDRKGSLQHCFTSVEEREGLWKERASRT
ERYRKQASNKIRSNRQMRNASSEEIETILDKRLSNSRNEYQKSEKVIRRYRVQDAL
LFLLAKKTLTELADFDGERFKLKEIMPDAEKGILSEIMPMSFTFEKGGKKYTITSEG
MKLKNYGDFFVLASDKRIGNLLELVGSDIVSKEDIMEEFNKYDQCRPEISSIVENLE
KWAFDTYPELSARVDREEKVDFKSILKILLNNKNINKEQSDILRKIRNAFDANNYP
DKGVVEIKALPEIAMSIKKAFGEYAIMKGSLQLPPLERLTLQLHLPQVLADAVSRLV
IGKFGDLTDNFSSPHARRIGLAGVVMTTGTDVKDAKVICVSTGSKCINGEYLSDRGLALN
DCHAEIVSRRSLLRFLYTQLELYLNNEDDQKRSIFQKSERGGFRLKENIQFHLYISTSPCG
DARIFSPHEAILEEPADRHPNRKARGQLRTKIEAGQGTIPVRNNASIQTWDGVLQGERLL
TMSCSDKIARWNVVGIQGSLLSIFVEPIYFSSIILGSLYHGDHLSRAMYQRISNIEDLPPLYT
LNKPLLTGISNAEARQPGKAPIFSVNWTVGDSAIEVINATTGKGELGRASRLCKHALYCR
WMRVHGKVPSHLLRSKITKPNVYHETKLAAKEYQAAKARLFTAFIKAGLGAWVEKPTE
QDQFSLTP (SEQ ID NO: 67)
Regular: dPsp-Cas13b
Bold: GS-NES
Italics: RESCUE (human ADAR2 Deaminase Domain evolved from wt hADAR2
Deaminase Domain)

Various species exhibit codon bias (i.e. differences in codon usage by organisms) which correlates with the efficiency of translation of messenger RNA (mRNA) by utilizing codons in mRNA that correspond with the abundance of tRNA species for that codon in a particular organism. Various methods in the art can be used for computer optimization, including for example through use of software. In some embodiments, codon optimization refers to modification of nucleic acid sequences for enhanced expression in the host cells of interest by replacing at least one codon (e.g. 1, 2, 3, 4, 5, 10, 15, 20, 25, 50 or more codons) of the native sequence with codons that are more frequently used or most frequently used in the genes of the host cell while maintaining the native amino acid sequence.

In some embodiments, the Cas protein described herein is codon optimized. This type of optimization is known in the art and entails the mutation of foreign-derived DNA to mimic the codon preferences of the intended host organism or cell while encoding the same Cas protein. Thus, the codons are changed, but the encoded protein remains unchanged. Codon optimization improves soluble protein levels and increases activity and editing efficiency in a given species. Codon optimization also results in increased translation and protein expression. In some embodiments, the Cas protein is codon optimized for expression in eukaryotic cells. In some embodiments, the Cas protein is codon optimized for expression in human cells.

In some embodiments, the Cas13 protein is fused to one or more heterologous protein domains. In some embodiments, the Cas13 protein is fused to more than about 1, 2, 3, 4, 5, 6, 7, 8, 9, 10 or more protein domains. In some embodiments, the heterologous protein domain is fused to the C-terminus of the Cas13 protein. In some embodiments, the heterologous protein domain is fused to the N-terminus of the Cas13 protein. In some embodiments, the heterologous protein domain is fused internally, between the C-terminus and the N-terminus of the Cas13 protein. In some embodiments, the internal fusion is made within the Cas13 RuvCI, RuvC II, RuvCIII, HNH, REC I, or PAM interacting domain.

A Cas13 protein may be directly or indirectly linked to another protein domain. In some embodiments, a suitable CRISPR system contains a linker or spacer that joins a Cas13 protein and a heterologous protein. An amino acid linker or spacer is generally designed to be flexible or to interpose a structure, such as an alpha-helix, between the two protein moieties. A linker or spacer can be relatively short, or can be longer. Typically, a linker or spacer contains for example 1-100 (e.g., 1-100, 5-100, 10-100, 20-100 30-100, 40-100, 50-100, 60-100, 70-100, 80-100, 90-100, 5-55, 10-50, 10-45, 10-40, 10-35, 10-30, 10-25, 10-20) amino acids in length. In some embodiments, a linker or spacer is equal to or longer than 1, 2, 3, 4, 5, 6, 7, 8, 9, 10, 15, 20, 25, 30, 35, 40, 45, 50, 55, 60, 65, 70, 75, 80, 85, 90, 95, or 100 amino acids in length. Typically, a longer linker may decrease steric hindrance. In some embodiments, a linker will comprise a mixture of glycine and serine residues. In some embodiments, the linker may additionally comprise threonine, proline and/or alanine residues.

In some embodiments, a Cas13 protein is fused to cellular localization signals, epitope tags, reporter genes, and protein domains with enzymatic activity, epigenetic modifying activity, RNA cleavage activity, nucleic acid binding activity, transcription modulation activity. In some embodiments, the Cas13 protein is fused to a nuclear localization sequence (NLS), a FLAG tag, a HIS tag, and/or a HA tag.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify nucleic acid or nucleic acid-associated polypeptide (e.g., a histone or transcription factor). In some embodiments, the Cas13 protein is fused to a histone demethylase, a transcriptional activator or a deaminase.

In particular embodiments, a Cas13 is fused to a base editor that comprises a cytidine or adenosine deaminase domain, e.g., for use in base editing. In some embodiments, the terms “cytidine deaminase” and “cytosine deaminase” can be used interchangeably. In certain embodiments, the cytidine deaminase domain may have sequence identity of 70%, 75%, 80%, 85%, 90%, 91%, 92%, 93%, 94%, 95%, 96%, 97%, 98%, 99% or more to any cytidine deaminase described herein. In some embodiments, the cytidine deaminase domain has cytidine deaminase activity, (e.g., converting C to U). In certain embodiments, the adenosine deaminase domain may have sequence identity of 70%, 75%, 80%, 85%, 90%, 91%, 92%, 93%, 94%, 95%, 96%, 97%, 98%, 99% or more to any adenosine deaminase described herein. In some embodiments, the adenosine deaminase domain has adenosine deaminase activity, (e.g., converting A to I). In some embodiments, the terms “adenosine deaminase” and “adenine deaminase” can be used interchangeably.

### Cytidine or Cytosine Deaminase

In some embodiments, a cytidine deaminase can comprise all or a portion of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase. In some embodiments, a deaminase incorporated into a fusion protein comprises all or a portion of an APOBEC1 deaminase. In some embodiments, a deaminase incorporated into a fusion protein comprises all or a portion of APOBEC2 deaminase. In some embodiments, a deaminase incorporated into a fusion protein comprises all or a portion of is an APOBEC3 deaminase. In some embodiments, a deaminase incorporated into a fusion protein comprises all or a portion of an APOBEC3A deaminase. In some embodiments, a deaminase incorporated into a fusion protein comprises all or a portion of APOBEC3B deaminase. In some embodiments, a deaminase incorporated into a fusion protein comprises all or a portion of APOBEC3C deaminase. In some embodiments, a deaminase incorporated into a fusion protein comprises all or a portion of APOBEC3D deaminase. In some embodiments, a deaminase incorporated into a fusion protein comprises all or a portion of APOBEC3E deaminase. In some embodiments, a deaminase incorporated into a fusion protein comprises all or a portion of APOBEC3F deaminase. In some embodiments, a deaminase incorporated into a fusion protein comprises all or a portion of APOBEC3G deaminase. In some embodiments, a deaminase incorporated into a fusion protein comprises all or a portion of APOBEC3H deaminase. In some embodiments, a deaminase incorporated into a fusion protein comprises all or a portion of APOBEC4 deaminase. In some embodiments, a deaminase incorporated into a fusion protein comprises all or a portion of activation-induced deaminase (AID). In some embodiments a deaminase incorporated into a fusion protein comprises all or a portion of cytidine deaminase 1 (CDA1). It should be appreciated that a fusion protein can comprise a deaminase from any suitable organism (e.g., a human or a rat). In some embodiments, a deaminase domain of a fusion protein is from a human, chimpanzee, gorilla, monkey, cow, dog, rat, or mouse. In some embodiments, the deaminase domain of the fusion protein is derived from rat (e.g., rat APOBEC1). In some embodiments, the deaminase domain is human APOBEC1. In some embodiments, the deaminase domain is pmCDA1.

In some embodiments, Cas13 comprises a ppAPOBEC1 cytidine deaminase fused to the N-terminus of Cas13. In some embodiments, the Cas13 ppAPOBEC1 fusion further comprises a nuclear localization sequence (NLS) and a linker sequence.

Sequences of exemplary cytidine deaminases are provided below.

pmCDA1 (Petromyzon marinus)
(SEQ ID NO: 69)
MTDAEYVRIHEKLDIYTFKKQFFNNKKSVSHRCYVLFELKRRGERRACFWGYAVNK
PQSGTERGIHAEIFSIRKVEEYLRDNPGQFTINWYSSWSPCADCAEKILEWYNQELRG
NGHTLKIWACKLYYEKNARNQIGLWNLRDNGVGLNVMVSEHYQCCRKIFIQSSHNQ
LNENRWLEKTLKRAEKRRSELSIMIQVKILHTTKSPAV
Human AID:
(SEQ ID NO: 70)
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTAR
LYFCEDRKAEPEGLRRLHRAGVQIAIMTFKAPV
Human AID:
(SEQ ID NO: 71)
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTAR
LYFCEDRKAEPEGLRRLHRAGVQIAIMTFKDYFYCWNTFVENHERTFKAWEGLHEN
SVRLSRQLRRILLPLYEVDDLRDAFRTLGL
(underline: nuclear localization sequence; double underline: nuclear export signal)
Mouse AID:
(SEQ ID NO: 72)
MDSLLMKQKKFLYHFKNVRWAKGRHETYLCYVVKRRDSATSCSLDFGHLRNKSGC
HVELLFLRYISDWDLDPGRCYRVTWFTSWSPCYDCARHVAEFLRWNPNLSLRIFTAR
LYFCEDRKAEPEGLRRLHRAGVQIGIMTFKDYFYCWNTFVENRERTFKAWEGLHEN
SVRLTRQLRRILLPLYEVDDLRDAFRMLGF
(underline: nuclear localization sequence; double underline: nuclear export signal)
Canine AID:
(SEQ ID NO: 73)
MDSLLMKQRKFLYHFKNVRWAKGRHETYLCYVVKRRDSATSFSLDFGHLRNKSGC
HVELLFLRYISDWDLDPGRCYRVTWFTSWSPCYDCARHVADFLRGYPNLSLRIFAAR
LYFCEDRKAEPEGLRRLHRAGVQIAIMTFKDYFYCWNTFVENREKTFKAWEGLHEN
SVRLSRQLRRILLPLYEVDDLRDAFRTLGL
(underline: nuclear localization sequence; double underline: nuclear export signal)
Bovine AID:
(SEQ ID NO: 74)
MDSLLKKQRQFLYQFKNVRWAKGRHETYLCYVVKRRDSPTSFSLDFGHLRNKAGC
HVELLFLRYISDWDLDPGRCYRVTWFTSWSPCYDCARHVADFLRGYPNLSLRIFTAR
LYFCDKERKAEPEGLRRLHRAGVQIAIMTFKDYFYCWNTFVENHERTFKAWEGLHE
NSVRLSRQLRRILLPLYEVDDLRDAFRTLGL
(underline: nuclear localization sequence; double underline: nuclear export signal)
Rat AID:
(SEQ ID NO: 75)
MAVGSKPKAALVGPHWERERIWCFLCSTGLGTQQTGQTSRWLRPAATQDPVSPPRS
LLMKQRKFLYHFKNVRWAKGRHETYLCYVVKRRDSATSFSLDFGYLRNKSGCHVE
LLFLRYISDWDLDPGRCYRVTWFTSWSPCYDCARHVADFLRGNPNLSLRIFTARLTG
WGALPAGLMSPARPSDYFYCWNTFVENHERTFKAWEGLHENSVRLSRRLRRILLPL
YEVDDLRDAFRTLGL
(underline: nuclear localization sequence; double underline: nuclear export signal)
clAID (Canis lupus familiaris):
(SEQ ID NO: 76)
MDSLLMKQRKFLYHFKNVRWAKGRHETYLCYVVKRRDSATSFSLDFGHLRNKSGC
HVELLFLRYISDWDLDPGRCYRVTWFTSWSPCYDCARHVADFLRGYPNLSLRIFAAR
LYFCEDRKAEPEGLRRLHRAGVQIAIMTFKDYFYCWNTFVENREKTFKAWEGLHEN
SVRLSRQLRRILLPLYEVDDLRDAFRTLGL
btAID (Bos Taurus):
(SEQ ID NO: 77)
MDSLLKKQRQFLYQFKNVRWAKGRHETYLCYVVKRRDSPTSFSLDFGHLRNKAGC
HVELLFLRYISDWDLDPGRCYRVTWFTSWSPCYDCARHVADFLRGYPNLSLRIFTAR
LYFCDKERKAEPEGLRRLHRAGVQIAIMTFKDYFYCWNTFVENHERTFKAWEGLHE
NSVRLSRQLRRILLPLYEVDDLRDAFRTLGL
mAID (Mus musculus):
(SEQ ID NO: 78)
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTAR
LYFCEDRKAEPEGLRRLHRAGVQIAIMTFKDYFYCWNTFVENHERTFKAWEGLHEN
SVRLSRQLRRILLPLYEVDDLRDAFRTLGL
rAPOBEC-1 (Rattus norvegicus):
(SEQ ID NO: 79)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
maAPOBEC-1 (Mesocricetus auratus):
(SEQ ID NO: 80)
MSSETGPVVVDPTLRRRIEPHEFDAFFDQGELRKETCLLYEIRWGGRHNIWRHTGQN
TSRHVEINFIEKFTSERYFYPSTRCSIVWFLSWSPCGECSKAITEFLSGHPNVTLFIYAA
RLYHHTDQRNRQGLRDLISRGVTIRIMTEQEYCYCWRNFVNYPPSNEVYWPRYPNL
WMRLYALELYCIHLGLPPCLKIKRRHQYPLTFFRLNLQSCHYQRIPPHILWATGFI
ppAPOBEC-1 (Pongo pygmaeus):
(SEQ ID NO: 81)
MTSEKGPSTGDPTLRRRIESWEFDVFYDPRELRKETCLLYEIKWGMSRKIWRSSGKN
TTNHVEVNFIKKFTSERRFHSSISCSITWFLSWSPCWECSQAIREFLSQHPGVTLVIYV
ARLFWHMDQRNRQGLRDLVNSGVTIQIMRASEYYHCWRNFVNYPPGDEAHWPQYP
PLWMMLYALELHCIILSLPPCLKISRRWQNHLAFFRLHLQNCHYQTIPPHILLATGLIH
PSVTWR
OCAPOBEC1 (Oryctolagus cuniculus):
(SEQ ID NO: 82)
MASEKGPSNKDYTLRRRIEPWEFEVFFDPQELRKEACLLYEIKWGASSKTWRSSGKN
TTNHVEVNFLEKLTSEGRLGPSTCCSITWFLSWSPCWECSMAIREFLSQHPGVTLIIFV
ARLFQHMDRRNRQGLKDLVTSGVTVRVMSVSEYCYCWENFVNYPPGKAAQWPRY
PPRWMLMYALELYCIILGLPPCLKISRRHQKQLTFFSLTPQYCHYKMIPPYILLATGLL
QPSVPWR
mdAPOBEC-1 (Monodelphis domestica):
(SEQ ID NO: 83)
MNSKTGPSVGDATLRRRIKPWEFVAFFNPQELRKETCLLYEIKWGNQNIWRHSNQN
TSQHAEINFMEKFTAERHFNSSVRCSITWFLSWSPCWECSKAIRKFLDHYPNVTLAIFI
SRLYWHMDQQHRQGLKELVHSGVTIQIMSYSEYHYCWRNFVDYPQGEEDYWPKYP
YLWIMLYVLELHCIILGLPPCLKISGSHSNQLALFSLDLQDCHYQKIPYNVLVATGLV
QPFVTWR
ppAPOBEC-2 (Pongo pygmaeus):
(SEQ ID NO: 84)
MAQKEEAAAATEAASQNGEDLENLDDPEKLKELIELPPFEIVTGERLPANFFKFQFRN
VEYSSGRNKTFLCYVVEAQGKGGQVQASRGYLEDEHAAAHAEEAFFNTILPAFDPA
LRYNVTWYVSSSPCAACADRIIKTLSKTKNLRLLILVGRLFMWEELEIQDALKKLKE
AGCKLRIMKPQDFEYVWQNFVEQEEGESKAFQPWEDIQENFLYYEEKLADILK
btAPOBEC-2 (Bos Taurus):
(SEQ ID NO: 85)
MAQKEEAAAAAEPASQNGEEVENLEDPEKLKELIELPPFEIVTGERLPAHYFKFQFRN
VEYSSGRNKTFLCYVVEAQSKGGQVQASRGYLEDEHATNHAEEAFFNSIMPTFDPA
LRYMVTWYVSSSPCAACADRIVKTLNKTKNLRLLILVGRLFMWEEPEIQAALRKLKE
AGCRLRIMKPQDFEYIWQNFVEQEEGESKAFEPWEDIQENFLYYEEKLADILK
mAPOBEC-3-(1) (Mus musculus):
(SEQ ID NO: 86)
MQPQRLGPRAGMGPFCLGCSHRKCYSPIRNLISQETFKFHFKNLGYAKGRKDTFLCY
EVTRKDCDSPVSLHHGVFKNKDNIHAEICFLYWFHDKVLKVLSPREEFKITWYMSW
SPCFECAEQIVRFLATHHNLSLDIFSSRLYNVQDPETQQNLCRLVQEGAQVAAMDLY
EFKKCWKKFVDNGGRRFRPWKRLLTNFRYQDSKLQEILRPCYISVPSSSSSTLSNICL
TKGLPETRFWVEGRRMDPLSEEEFYSQFYNQRVKHLCYYHRMKPYLCYQLEQFNG
QAPLKGCLLSEKGKQHAEILFLDKIRSMELSQVTITCYLTWSPCPNCAWQLAAFKRD
RPDLILHIYTSRLYFHWKRPFQKGLCSLWQSGILVDVMDLPQFTDCWTNFVNPKRPF
WPWKGLEIISRRTQRRLRRIKESWGLQDLVNDFGNLQLGPPMS
Mouse APOBEC-3-(2):
(SEQ ID NO: 87)
MGPFCLGCSHRKCYSPIRNLISQETFKFHFKNLGYAKGRKDTFLCYEVTRKDCDSPV
SLHHGVFKNKDNIHAEICFLYWFHDKVLKVLSPREEFKITWYMSWSPCFECAEQIVRFL
ATHHNLSLDIFSSRLYNVQDPETQQNLCRLVQEGAQVAAMDLYEFKKCWKKFVDN
GGRRFRPWKRLLTNFRYQDSKLQEILRPCYIPVPSSSSSTLSNICLTKGLPETRFCVEG
RRMDPLSEEEFYSQFYNQRVKHLCYYHRMKPYLCYQLEQFNGQAPLKGCLLSEKGK
QHAEILFLDKIRSMELSQVTITCYLTWSPCPNCAWQLAAFKRDRPDLILHIYTSRLYFHW
KRPFQKGLCSLWQSGILVDVMDLPQFTDCWTNFVNPKRPFWPWKGLEIISRRTQRRL
RRIKESWGLQDLVNDFGNLQLGPPMS
(italic: nucleic acid editing domain)
Rat APOBEC-3:
(SEQ ID NO: 88)
MGPFCLGCSHRKCYSPIRNLISQETFKFHFKNRLRYAIDRKDTFLCYEVTRKDCDSPV
SLHHGVFKNKDNIHAEICFLYWFHDKVLKVLSPREEFKITWYMSWSPCFECAEQVLRFL
ATHHNLSLDIFSSRLYNIRDPENQQNLCRLVQEGAQVAAMDLYEFKKCWKKFVDNG
GRRFRPWKKLLTNFRYQDSKLQEILRPCYIPVPSSSSSTLSNICLTKGLPETRFCVERR
RVHLLSEEEFYSQFYNQRVKHLCYYHGVKPYLCYQLEQFNGQAPLKGCLLSEKGKQ
HAEILFLDKIRSMELSQVIITCYLTWSPCPNCAWQLAAFKRDRPDLILHIYTSRLYFHWK
RPFQKGLCSLWQSGILVDVMDLPQFTDCWTNFVNPKRPFWPWKGLEIISRRTQRRLH
RIKESWGLQDLVNDFGNLQLGPPMS
(italic: nucleic acid editing domain)
hAPOBEC-3A (Homo sapiens):
(SEQ ID NO: 89)
MEASPASGPRHLMDPHIFTSNFNNGIGRHKTYLCYEVERLDNGTSVKMDQHRGFLH
NQAKNLLCGFYGRHAELRFLDLVPSLQLDPAQIYRVTWFISWSPCFSWGCAGEVRAF
LQENTHVRLRIFAARIYDYDPLYKEALQMLRDAGAQVSIMTYDEFKHCWDTFVDHQ
GCPFQPWDGLDEHSQALSGRLRAILQNQGN
hAPOBEC-3F (Homo sapiens):
(SEQ ID NO: 90)
MKPHFRNTVERMYRDTFSYNFYNRPILSRRNTVWLCYEVKTKGPSRPRLDAKIFRGQ
VYSQPEHHAEMCFLSWFCGNQLPAYKCFQITWFVSWTPCPDCVAKLAEFLAEHPNV
TLTISAARLYYYWERDYRRALCRLSQAGARVKIMDDEEFAYCWENFVYSEGQPFMP
WYKFDDNYAFLHRTLKEILRNPMEAMYPHIFYFHFKNLRKAYGRNESWLCFTMEV
VKHHSPVSWKRGVFRNQVDPETHCHAERCFLSWFCDDILSPNTNYEVTWYTSWSPC
PECAGEVAEFLARHSNVNLTIFTARLYYFWDTDYQEGLRSLSQEGASVEIMGYKDFK
YCWENFVYNDDEPFKPWKGLKYNFLFLDSKLQEILE
Rhesus macaque APOBEC-3G:
(SEQ ID NO: 91)
MVEPMDPRTFVSNFNNRPILSGLNTVWLCCEVKTKDPSGPPLDAKIFOGKVYSKAKY
HPEMRFLRWFHKWRQLHHDQEYKVTWYVSWSPCTRCANSVATFLAKDPKVTLTIF
VARLYYFWKPDYQQALRILCQKRGGPHATMKIMNYNEFQDCWNKFVDGRGKPFKP
RNNLPKHYTLLQATLGELLRHLMDPGTFTSNFNNKPWVSGQHETYLCYKVERLHND
TWVPLNQHRGFLRNQAPNIHGFPKGRHAELCFLDLIPFWKLDGQQYRVTCFTSWSPC
FSCAQEMAKFISNNEHVSLCIFAARIYDDQGRYQEGLRALHRDGAKIAMMNYSEFEY
CWDTFVDRQGRPFQPWDGLDEHSQALSGRLRAI
(italic: nucleic acid editing domain; underline: cytoplasmic localization signal)
Chimpanzee APOBEC-3G:
(SEQ ID NO: 92)
MKPHERNPVERMYQDTFSDNFYNRPILSHRNTVWLCYEVKTKGPSRPPLDAKIFRGQ
VYSKLKYHPEMRFFHWFSKWRKLHRDQEYEVTWYISWSPCTKCTRDVATFLAEDPKV
TLTIFVARLYYFWDPDYQEALRSLCQKRDGPRATMKIMNYDEFQHCWSKFVYSQRE
LFEPWNNLPKYYILLHIMLGEILRHSMDPPTFTSNFNNELWVRGRHETYLCYEVERL
HNDTWVLLNQRRGFLCNQAPHKHGFLEGRHAELCFLDVIPFWKLDLHQDYRVTCFTS
WSPCFSCAQEMAKFISNNKHVSLCIFAARIYDDQGRCQEGLRTLAKAGAKISIMTYSE
FKHCWDTFVDHQGCPFQPWDGLEEHSQALSGRLRAILQNQGN
(italic: nucleic acid editing domain; underline: cytoplasmic localization signal)
Green monkey APOBEC-3G:
(SEQ ID NO: 93)
MNPQIRNMVEQMEPDIFVYYFNNRPILSGRNTVWLCYEVKTKDPSGPPLDANIFQGK
LYPEAKDHPEMKFLHWFRKWRQLHRDQEYEVTWYVSWSPCTRCANSVATFLAEDPKV
TLTIFVARLYYFWKPDYQQALRILCQERGGPHATMKIMNYNEFQHCWNEFVDGQG
KPFKPRKNLPKHYTLLHATLGELLRHVMDPGTFTSNFNNKPWVSGQRETYLCYKVE
RSHNDTWVLLNQHRGFLRNQAPDRHGFPKGRHAELCFLDLIPFWKLDDQQYRVTCFT
SWSPCFSCAQKMAKFISNNKHVSLCIFAARIYDDQGRCQEGLRTLHRDGAKIAVMNY
SEFEYCWDTFVDRQGRPFQPWDGLDEHSQALSGRLRAI
(italic: nucleic acid editing domain; underline: cytoplasmic localization signal)
Human APOBEC-3G:
(SEQ ID NO: 94)
MKPHFRNTVERMYRDTFSYNFYNRPILSRRNTVWLCYEVKTKGPSRPPLDAKIFRGQ
VYSELKYHPEMRFFHWFSKWRKLHRDQEYEVTWYISWSPCTKCTRDMATFLAEDPKV
TLTIFVARLYYFWDPDYQEALRSLCQKRDGPRATMKIMNYDEFQHCWSKFVYSQRE
LFEPWNNLPKYYILLHIMLGEILRHSMDPPTFTFNFNNEPWVRGRHETYLCYEVERM
HNDTWVLLNQRRGFLCNQAPHKHGFLEGRHAELCFLDVIPFWKLDLDQDYRVTCFTS
WSPCFSCAQEMAKFISKNKHVSLCIFTARIYDDQGRCQEGLRTLAEAGAKISIMTYSE
FKHCWDTFVDHQGCPFQPWDGLDEHSQDLSGRLRAILQNQEN
(italic: nucleic acid editing domain; underline: cytoplasmic localization signal)
Human APOBEC-3F:
(SEQ ID NO: 95)
MKPHFRNTVERMYRDTFSYNFYNRPILSRRNTVWLCYEVKTKGPSRPRLDAKIFRGQ
VYSQPEHHAEMCFLSWFCGNQLPAYKCFQITWFVSWTPCPDCVAKLAEFLAEHPNVTL
TISAARLYYYWERDYRRALCRLSQAGARVKIMDDEEFAYCWENFVYSEGQPFMPW
YKFDDNYAFLHRTLKEILRNPMEAMYPHIFYFHFKNLRKAYGRNESWLCFTMEVVK
HHSPVSWKRGVFRNQVDPETHCHAERCFLSWFCDDILSPNTNYEVTWYTSWSPCPECA
GEVAEFLARHSNVNLTIFTARLYYFWDTDYQEGLRSLSQEGASVEIMGYKDFKYCW
ENFVYNDDEPFKPWKGLKYNFLFLDSKLQEILE
(italic: nucleic acid editing domain)
Human APOBEC-3B:
(SEQ ID NO: 96)
MNPQIRNPMERMYRDTFYDNFENEPILYGRSYTWLCYEVKIKRGRSNLLWDTGVFR
GQVYFKPQYHAEMCFLSWFCGNQLPAYKCFQITWFVSWTPCPDCVAKLAEFLSEHPN
VTLTISAARLYYYWERDYRRALCRLSQAGARVTIMDYEEFAYCWENFVYNEGQQF
MPWYKFDENYAFLHRTLKEILRYLMDPDTFTFNFNNDPLVLRRRQTYLCYEVERLD
NGTWVLMDQHMGFLCNEAKNLLCGFYGRHAELRFLDLVPSLQLDPAQIYRVTWFISWS
PCFSWGCAGEVRAFLQENTHVRLRIFAARIYDYDPLYKEALQMLRDAGAQVSIMTY
DEFEYCWDTFVYRQGCPFQPWDGLEEHSQALSGRLRAILQNQGN
(italic: nucleic acid editing domain)
Rat APOBEC-3B:
(SEQ ID NO: 97)
MQPQGLGPNAGMGPVCLGCSHRRPYSPIRNPLKKLYQQTFYFHFKNVRYAWGRKN
NFLCYEVNGMDCALPVPLRQGVFRKQGHIHAELCFIYWFHDKVLRVLSPMEEFKVT
WYMSWSPCSKCAEQVARFLAAHRNLSLAIFSSRLYYYLRNPNYQQKLCRLIQEGVH
VAAMDLPEFKKCWNKFVDNDGQPFRPWMRLRINFSFYDCKLQEIFSRMNLLREDVF
YLQFNNSHRVKPVQNRYYRRKSYLCYQLERANGQEPLKGYLLYKKGEQHVEILFLE
KMRSMELSQVRITCYLTWSPCPNCARQLAAFKKDHPDLILRIYTSRLYFWRKKFQKG
LCTLWRSGIHVDVMDLPQFADCWTNFVNPQRPFRPWNELEKNSWRIQRRLRRIKES
WGL
Bovine APOBEC-3B:
(SEQ ID NO: 98)
MDGWEVAFRSGTVLKAGVLGVSMTEGWAGSGHPGQGACVWTPGTRNTMNLLREV
LFKQQFGNQPRVPAPYYRRKTYLCYQLKQRNDLTLDRGCFRNKKQRHAERFIDKIN
SLDLNPSQSYKIICYITWSPCPNCANELVNFITRNNHLKLEIFASRLYFHWIKSFKMGL
QDLQNAGISVAVMTHTEFEDCWEQFVDNQSRPFQPWDKLEQYSASIRRRLQRILTAPI
Chimpanzee APOBEC-3B:
(SEQ ID NO: 99)
MNPQIRNPMEWMYQRTFYYNFENEPILYGRSYTWLCYEVKIRRGHSNLLWDTGVFR
GQMYSQPEHHAEMCFLSWFCGNQLSAYKCFQITWFVSWTPCPDCVAKLAKFLAEH
PNVTLTISAARLYYYWERDYRRALCRLSQAGARVKIMDDEEFAYCWENFVYNEGQP
FMPWYKFDDNYAFLHRTLKEIIRHLMDPDTFTFNFNNDPLVLRRHQTYLCYEVERLD
NGTWVLMDQHMGFLCNEAKNLLCGFYGRHAELRFLDLVPSLQLDPAQIYRVTWFIS
WSPCFSWGCAGQVRAFLQENTHVRLRIFAARIYDYDPLYKEALQMLRDAGAQVSIM
TYDEFEYCWDTFVYRQGCPFQPWDGLEEHSQALSGRLRAILQVRASSLCMVPHRPPP
PPQSPGPCLPLCSEPPLGSLLPTGRPAPSLPFLLTASFSFPPPASLPPLPSLSLSPGHLPVP
SFHSLTSCSIQPPCSSRIRETEGWASVSKEGRDLG
Human APOBEC-3C:
(SEQ ID NO: 100)
MNPQIRNPMKAMYPGTFYFQFKNLWEANDRNETWLCFTVEGIKRRSVVSWKTGVF
RNQVDSETHCHAERCFLSWFCDDILSPNTKYQVTWYTSWSPCPDCAGEVAEFLARHSN
VNLTIFTARLYYFQYPCYQEGLRSLSQEGVAVEIMDYEDFKYCWENFVYNDNEPFKP
WKGLKTNFRLLKRRLRESLQ
(italic: nucleic acid editing domain)
Gorilla APOBEC-3C
(SEQ ID NO: 101)
MNPQIRNPMKAMYPGTFYFQFKNLWEANDRNETWLCFTVEGIKRRSVVSWKTGVF
RNQVDSETHCHAERCFLSWECDDILSPNTNYQVTWYTSWSPCPECAGEVAEFLARHSN
VNLTIFTARLYYFQDTDYQEGLRSLSQEGVAVKIMDYKDFKYCWENFVYNDDEPFK
PWKGLKYNFRFLKRRLQEILE
(italic: nucleic acid editing domain)
Human APOBEC-3A:
(SEQ ID NO: 102)
MEASPASGPRHLMDPHIFTSNFNNGIGRHKTYLCYEVERLDNGTSVKMDQHRGFLH
NQAKNLLCGFYGRHAELRFLDLVPSLQLDPAQIYRVTWFISWSPCFSWGCAGEVRAFLQ
ENTHVRLRIFAARIYDYDPLYKEALQMLRDAGAQVSIMTYDEFKHCWDTFVDHQGC
PFQPWDGLDEHSQALSGRLRAILQNQGN
(italic: nucleic acid editing domain)
Rhesus macaque APOBEC-3A:
(SEQ ID NO: 103)
MDGSPASRPRHLMDPNTFTFNFNNDLSVRGRHQTYLCYEVERLDNGTWVPMDERR
GFLCNKAKNVPCGDYGCHVELRFLCEVPSWQLDPAQTYRVTWFISWSPCFRRGCAGQ
VRVFLQENKHVRLRIFAARIYDYDPLYQEALRTLRDAGAQVSIMTYEEFKHCWDTF
VDRQGRPFQPWDGLDEHSQALSGRLRAILQNQGN
(italic: nucleic acid editing domain)
Bovine APOBEC-3A:
(SEQ ID NO: 104)
MDEYTFTENFNNQGWPSKTYLCYEMERLDGDATIPLDEYKGFVRNKGLDQPEKPCH
AELYFLGKIHSWNLDRNQHYRLTCFISWSPCYDCAQKLTTFLKENHHISLHILASRIYTH
NRFGCHQSGLCELQAAGARITIMTFEDFKHCWETFVDHKGKPFQPWEGLNVKSQAL
CTELQAILKTQQN
(italic: nucleic acid editing domain)
Human APOBEC-3H:
(SEQ ID NO: 105)
MALLTAETFRLQFNNKRRLRRPYYPRKALLCYQLTPQNGSTPTRGYFENKKKCHAEI
CFINEIKSMGLDETQCYQVTCYLTWSPCSSCAWELVDFIKAHDHLNLGIFASRLYYHWC
KPQQKGLRLLCGSQVPVEVMGFPKFADCWENFVDHEKPLSFNPYKMLEELDKNSRA
IKRRLERIKIPGVRAQGRYMDILCDAEV
(italic: nucleic acid editing domain)
Rhesus macaque APOBEC-3H:
(SEQ ID NO: 106)
MALLTAKTFSLQFNNKRRVNKPYYPRKALLCYQLTPQNGSTPTRGHLKNKKKDHAE
IRFINKIKSMGLDETQCYQVTCYLTWSPCPSCAGELVDFIKAHRHLNLRIFASRLYYH
WRPNYQEGLLLLCGSQVPVEVMGLPEFTDCWENFVDHKEPPSFNPSEKLEELDKNS
QAIKRRLERIKSRSVDVLENGLRSLQLGPVTPSSSIRNSR
Human APOBEC-3D:
(SEQ ID NO: 107)
MNPQIRNPMERMYRDTFYDNFENEPILYGRSYTWLCYEVKIKRGRSNLLWDTGVFR
GPVLPKRQSNHRQEVYFRFENHAEMCFLSWFCGNRLPANRRFQITWFVSWNPCLPCVV
KVTKFLAEHPNVTLTISAARLYYYRDRDWRWVLLRLHKAGARVKIMDYEDFAYCW
ENFVCNEGQPFMPWYKFDDNYASLHRTLKEILRNPMEAMYPHIFYFHFKNLLKACG
RNESWLCFTMEVTKHHSAVFRKRGVFRNQVDPETHCHAERCFLSWFCDDILSPNTNY
EVTWYTSWSPCPECAGEVAEFLARHSNVNLTIFTARLCYFWDTDYQEGLCSLSQEGAS
VKIMGYKDFVSCWKNFVYSDDEPFKPWKGLQTNFRLLKRRLREILQ
(italic: nucleic acid editing domain)
Human APOBEC-1:
(SEQ ID NO: 108)
MTSEKGPSTGDPTLRRRIEPWEFDVFYDPRELRKEACLLYEIKWGMSRKIWRSSGKN
TTNHVEVNFIKKFTSERDFHPSMSCSITWFLSWSPCWECSQAIREFLSRHPGVTLVIYV
ARLFWHMDQQNRQGLRDLVNSGVTIQIMRASEYYHCWRNFVNYPPGDEAHWPQY
PPLWMMLYALELHCIILSLPPCLKISRRWQNHLTFFRLHLQNCHYQTIPPHILLATGLI
HPSVAWR
Mouse APOBEC-1:
(SEQ ID NO: 109)
MSSETGPVAVDPTLRRRIEPHEFEVFFDPRELRKETCLLYEINWGGRHSVWRHTSQN
TSNHVEVNFLEKFTTERYFRPNTRCSITWFLSWSPCGECSRAITEFLSRHPYVTLFIYIA
RLYHHTDQRNRQGLRDLISSGVTIQIMTEQEYCYCWRNFVNYPPSNEAYWPRYPHL
WVKLYVLELYCIILGLPPCLKILRRKQPQLTFFTITLQTCHYQRIPPHLLWATGLK
Rat APOBEC-1:
(SEQ ID NO: 110)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
Human APOBEC-2:
(SEQ ID NO: 111)
MAQKEEAAVATEAASQNGEDLENLDDPEKLKELIELPPFEIVTGERLPANFFKFQFRN
VEYSSGRNKTFLCYVVEAQGKGGQVQASRGYLEDEHAAAHAEEAFFNTILPAFDPA
LRYNVTWYVSSSPCAACADRIIKTLSKTKNLRLLILVGRLFMWEEPEIQAALKKLKE
AGCKLRIMKPQDFEYVWQNFVEQEEGESKAFQPWEDIQENFLYYEEKLADILK
Mouse APOBEC-2:
(SEQ ID NO: 112)
MAQKEEAAEAAAPASQNGDDLENLEDPEKLKELIDLPPFEIVTGVRLPVNFFKFQFR
NVEYSSGRNKTFLCYVVEVQSKGGQAQATQGYLEDEHAGAHAEEAFFNTILPAFDP
ALKYNVTWYVSSSPCAACADRILKTLSKTKNLRLLILVSRLFMWEEPEVQAALKKLK
EAGCKLRIMKPQDFEYIWQNFVEQEEGESKAFEPWEDIQENFLYYEEKLADILK
Rat APOBEC-2:
(SEQ ID NO: 113)
MAQKEEAAEAAAPASQNGDDLENLEDPEKLKELIDLPPFEIVTGVRLPVNFFKFQFR
NVEYSSGRNKTFLCYVVEAQSKGGQVQATQGYLEDEHAGAHAEEAFFNTILPAFDP
ALKYNVTWYVSSSPCAACADRILKTLSKTKNLRLLILVSRLFMWEEPEVQAALKKLK
EAGCKLRIMKPQDFEYLWQNFVEQEEGESKAFEPWEDIQENFLYYEEKLADILK
Bovine APOBEC-2:
(SEQ ID NO: 114)
MAQKEEAAAAAEPASQNGEEVENLEDPEKLKELIELPPFEIVTGERLPAHYFKFQFRN
VEYSSGRNKTFLCYVVEAQSKGGQVQASRGYLEDEHATNHAEEAFFNSIMPTFDPA
LRYMVTWYVSSSPCAACADRIVKTLNKTKNLRLLILVGRLFMWEEPEIQAALRKLKE
AGCRLRIMKPQDFEYIWQNFVEQEEGESKAFEPWEDIQENFLYYEEKLADILK
Petromyzon marinus CDA1 (pmCDA1):
(SEQ ID NO: 115)
MTDAEYVRIHEKLDIYTFKKQFFNNKKSVSHRCYVLFELKRRGERRACFWGYAVNK
PQSGTERGIHAEIFSIRKVEEYLRDNPGQFTINWYSSWSPCADCAEKILEWYNQELRG
NGHTLKIWACKLYYEKNARNQIGLWNLRDNGVGLNVMVSEHYQCCRKIFIQSSHNQ
LNENRWLEKTLKRAEKRRSELSFMIQVKILHTTKSPAV
Human APOBEC3G D316R D317R:
(SEQ ID NO: 116)
MKPHFRNTVERMYRDTFSYNFYNRPILSRRNTVWLCYEVKTKGPSRPPLDAKIFRGQ
VYSELKYHPEMRFFHWFSKWRKLHRDQEYEVTWYISWSPCTKCTRDMATFLAEDP
KVTLTIFVARLYYFWDPDYQEALRSLCQKRDGPRATMKFNYDEFQHCWSKFVYSQ
RELFEPWNNLPKYYILLHFMLGEILRHSMDPPTFTFNFNNEPWVRGRHETYLCYEVE
RMHNDTWVLLNQRRGFLCNQAPHKHGFLEGRHAELCFLDVIPFWKLDLDQDYRVT
CFTSWSPCFSCAQEMAKFISKKHVSLCIFTARIYRRQGRCQEGLRTLAEAGAKISFTY
SEFKHCWDTFVDHQGCPFQPWDGLDEHSQDLSGRLRAILQNQEN
Human APOBEC3G chain A:
(SEQ ID NO: 117)
MDPPTFTFNFNNEPWWGRHETYLCYEVERMHNDTWVLLNQRRGFLCNQAPHKHGF
LEGRHAELCFLDVIPFWKLDLDQDYRVTCFTSWSPCFSCAQEMAKFISKNKHVSLCIF
TARIYDDQGRCQEGLRTLAEAGAKISFTYSEFKHCWDTFVDHQGCPFQPWDGLD
EHSQDLSGRLRAILQ
Human APOBEC3G chain A D120R D121R:
(SEQ ID NO: 118)
MDPPTFTFNFNNEPWVRGRHETYLCYEVERMHNDTWVLLNQRRGFLCNQAPHKHG
FLEGRHAELCFLDVIPFWKLDLDQDYRVTCFTSWSPCFSCAQEMAKFISKNKHVSLCI
FTARIYRRQGRCQEGLRTLAEAGAKISFMTYSEFKHCWDTFVDHQGCPFQPWDGLD
EHSQDLSGRLRAILQ
hAPOBEC-4 (Homo sapiens):
(SEQ ID NO: 119)
MEPIYEEYLANHGTIVKPYYWLSFSLDCSNCPYHIRTGEEARVSLTEFCQIFGFPYGTT
FPQTKHLTFYELKTSSGSLVQKGHASSCTGNYIHPESMLFEMNGYLDSAIYNNDSIRH
IILYSNNSPCNEANHCCISKMYNFLITYPGITLSIYFSQLYHTEMDFPASAWNREALRS
LASLWPRVVLSPISGGIWHSVLHSFISGVSGSHVFQPILTGRALADRHNAYEINAITGV
KPYFTDVLLQTKRNPNTKAQEALESYPLNNAFPGQFFQMPSGQLQPNLPPDLRAPVV
FVLVPLRDLPPMHMGQNPNKPRNIVRHLNMPQMSFQETKDLGRLPTGRSVEIVEITE
QFASSKEADEKKKKKGKK
mAPOBEC-4 (Mus musculus):
(SEQ ID NO: 120)
MDSLLMKQKKFLYHFKNVRWAKGRHETYLCYVVKRRDSATSCSLDFGHLRNKSGC
HVELLFLRYISDWDLDPGRCYRVTWFTSWSPCYDCARHVAEFLRWNPNLSLRIFTAR
LYFCEDRKAEPEGLRRLHRAGVQIGIMTFKDYFYCWNTFVENRERTFKAWEGLHEN
SVRLTRQLRRILLPLYEVDDLRDAFRMLGF
rAPOBEC-4 (Rattus norvegicus):
(SEQ ID NO: 121)
MEPLYEEYLTHSGTIVKPYYWLSVSLNCTNCPYHIRTGEEARVPYTEFHQTFGFPWST
YPQTKHLTFYELRSSSGNLIQKGLASNCTGSHTHPESMLFERDGYLDSLIFHDSNIRHI
ILYSNNSPCDEANHCCISKMYNFLMNYPEVTLSVFFSQLYHTENQFPTSAWNREALR
GLASLWPQVTLSAISGGIWQSILETFVSGISEGLTAVRPFTAGRTLTDRYNAYEINCIT
EVKPYFTDALHSWQKENQDQKVWAASENQPLHNTTPAQWQPDMSQDCRTPAVFM
LVPYRDLPPIHVNPSPQKPRTVVRHLNTLQLSASKVKALRKSPSGRPVKKEEARKGS
TRSQEANETNKSKWKKQTLFIKSNICHLLEREQKKIGILSSWSV
mfAPOBEC-4 (Macaca fascicularis):
(SEQ ID NO: 122)
MEPTYEEYLANHGTIVKPYYWLSFSLDCSNCPYHIRTGEEARVSLTEFCQIFGFPYGT
TYPQTKHLTFYELKTSSGSLVQKGHASSCTGNYIHPESMLFEMNGYLDSAIYNNDSIR
HIILYCNNSPCNEANHCCISKVYNFLITYPGITLSIYFSQLYHTEMDFPASAWNREALR
SLASLWPRVVLSPISGGIWHSVLHSFVSGVSGSHVFQPILTGRALTDRYNAYEINAITG
VKPFFTDVLLHTKRNPNTKAQMALESYPLNNAFPGQSFQMTSGIPPDLRAPVVFVLL
PLRDLPPMHMGQDPNKPRNIIRHLNMPQMSFQETKDLERLPTRRSVETVEITERFASS
KQAEEKTKKKKGKK
pmCDA-1 (Petromyzon marinus):
(SEQ ID NO: 123)
MAGYECVRVSEKLDFDTFEFQFENLHYATERHRTYVIFDVKPQSAGGRSRRLWGYII
NNPNVCHAELILMSMIDRHLESNPGVYAMTWYMSWSPCANCSSKLNPWLKNLLEE
QGHTLTMHFSRIYDRDREGDHRGLRGLKHVSNSFRMGVVGRAEVKECLAEYVEAS
RRTLTWLDTTESMAAKMRRKLFCILVRCAGMRESGIPLHLFTLQTPLLSGRVVWWRV
pmCDA-2 (Petromyzon marinus):
(SEQ ID NO: 124)
MELREVVDCALASCVRHEPLSRVAFLRCFAAPSQKPRGTVILFYVEGAGRGVTGGH
AVNYNKQGTSIHAEVLLLSAVRAALLRRRRCEDGEEATRGCTLHCYSTYSPCRDCVE
YIQEFGASTGVRVVIHCCRLYELDVNRRRSEAEGVLRSLSRLGRDFRLMGPRDAIAL
LLGGRLANTADGESGASGNAWVTETNVVEPLVDMTGFGDEDLHAQVQRNKQIREA
YANYASAVSLMLGELHVDPDKFPFLAEFLAQTSVEPSGTPRETRGRPRGASSRGPEIG
RQRPADFERALGAYGLFLHPRIVSREADREEIKRDLIVVMRKHNYQGP
pmCDA-5 (Petromyzon marinus):
(SEQ ID NO: 125)
MAGDENVRVSEKLDFDTFEFQFENLHYATERHRTYVIFDVKPQSAGGRSRRLWGYII
NNPNVCHAELILMSMIDRHLESNPGVYAMTWYMSWSPCANCSSKLNPWLKNLLEE
QGHTLMMHFSRIYDRDREGDHRGLRGLKHVSNSFRMGVVGRAEVKECLAEYVEAS
RRTLTWLDTTESMAAKMRRKLFCILVRCAGMRESGMPLHLFT
yCD (Saccharomyces cerevisiae):
(SEQ ID NO: 126)
MVTGGMASKWDQKGMDIAYEEAALGYKEGGVPIGGCLINNKDGSVLGRGHNMRF
QKGSATLHGEISTLENCGRLEGKVYKDTTLYTTLSPCDMCTGAIIMYGIPRCVVGEN
VNFKSKGEKYLQTRGHEVVVVDDERCKKIMKQFIDERPQDWFEDIGE
rAPOBEC-1 (delta 177-186):
(SEQ ID NO: 127)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLW
VRGLPPCLNILRRKQPQLTFFTIALQSCHYQRLPPHILWATGLK
rAPOBEC-1 (delta 202-213):
(SEQ ID NO: 128)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLW
VRLYVLELYCIILGLPPCLNILRRKQPQHYQRLPPHILWATGLK
Mouse APOBEC-3:
(SEQ ID NO: 129)
MGPFCLGCSHRKCYSPIRNLISQETFKFHFKNLGYAKGRKDTFLCYEVTRKDCDSPV
SLHHGVFKNKDNIHAEICFLYWFHDKVLKVLSPREEFKITWYMSWSPCFECAEQIVRFL
ATHHNLSLDIFSSRLYNVQDPETQQNLCRLVQEGAQVAAMDLYEFKKCWKKFVDN
GGRRFRPWKRLLTNFRYQDSKLQEILRPCYIPVPSSSSSTLSNICLTKGLPETRFCVEG
RRMDPLSEEEFYSQFYNQRVKHLCYYHRMKPYLCYQLEQFNGQAPLKGCLLSEKGK
QHAEILFLDKIRSMELSQVTITCYLTWSPCPNCAWQLAAFKRDRPDLILHIYTSRLYFHW
KRPFQKGLCSLWQSGILVDVMDLPQFTDCWTNFVNPKRPFWPWKGLEIISRRTQRRL
RRIKESWGLQDLVNDFGNLQLGPPMS
(italic: nucleic acid editing domain)

### Adenosine or Adenine Deaminase

In some embodiments, an adenosine deaminase can comprise all or a portion of an adenosine deaminase ADAR (e.g., ADAR1 or ADAR2). In another embodiment, an adenosine deaminase can comprise all or a portion of an adenosine deaminase ADAT. In some embodiments, an adenosine deaminase can comprise all or a portion of an ADAT from Escherichia coli (EcTadA) comprising one or more of the following mutations: D108N, A106V, D147Y, E155V, L84F, H123Y, 1157F, or a corresponding mutation in another adenosine deaminase. The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that corresponds to any of the mutations described herein (e.g., any of the mutations identified in ecTadA) can be generated accordingly. In particular embodiments, the TadA is any one of the TadA described in PCT/US2017/045381 (WO 2018/027078), which is incorporated herein by reference in its entirety. Mutations were identified through rounds of evolution and selection (e.g., TadA*7.10=variant 10 from seventh round of evolution) having desirable adenosine deaminase activity on single stranded DNA as shown in Table 6.

TABLE 6
Genotypes of TadA Variants

| TadA | 23 | 26 | 36 | 37 | 48 | 49 | 51 | 72 | 84 | 87 | 105 | 108 | 123 | 125 | 142 | 145 | 147 | 152 | 155 | 156 | 157 | 16 |
|---|---|---|---|---|---|---|---|---|---|---|---|---|---|---|---|---|---|---|---|---|---|---|
| 0.1 | W | R | H | N | P | | R | N | L | S | A | D | H | G | A | S | D | R | E | I | K | K |
| 0.2 | W | R | H | N | P | | R | N | L | S | A | D | H | G | A | S | D | R | E | I | K | K |
| 1.1 | W | R | H | N | P | | R | N | L | S | A | N | H | G | A | S | D | R | E | I | K | K |
| 1.2 | W | R | H | N | P | | R | N | L | S | V | N | H | G | A | S | D | R | E | I | K | K |
| 2.1 | W | R | H | N | P | | R | N | L | S | V | N | H | G | A | S | Y | R | V | I | K | K |
| 2.2 | W | R | H | N | P | | R | N | L | S | V | N | H | G | A | S | Y | R | V | I | K | K |
| 2.3 | W | R | H | N | P | | R | N | L | S | V | N | H | G | A | S | Y | R | V | I | K | K |
| 2.4 | W | R | H | N | P | | R | N | L | S | V | N | H | G | A | S | Y | R | V | I | K | K |
| 2.5 | W | R | H | N | P | | R | N | L | S | V | N | H | G | A | S | Y | R | V | I | K | K |
| 2.6 | W | R | H | N | P | | R | N | L | S | V | N | H | G | A | S | Y | R | V | I | K | K |
| 2.7 | W | R | H | N | P | | R | N | L | S | V | N | H | G | A | S | Y | R | V | I | K | K |
| 2.8 | W | R | H | N | P | | R | N | L | S | V | N | H | G | A | S | Y | R | V | I | K | K |
| 2.9 | W | R | H | N | P | | R | N | L | S | V | N | H | G | A | S | Y | R | V | I | K | K |
| 2.10 | W | R | H | N | P | | R | N | L | S | V | N | H | G | A | S | Y | R | V | I | K | K |
| 2.11 | W | R | H | N | P | | R | N | L | S | V | N | H | G | A | S | Y | R | V | I | K | K |
| 2.12 | W | R | H | N | P | | R | N | L | S | V | N | H | G | A | S | Y | R | V | I | K | K |
| 3.1 | W | R | H | N | P | | R | N | F | S | V | N | Y | G | A | S | Y | R | V | F | K | K |
| 3.2 | W | R | H | N | P | | R | N | F | S | V | N | Y | G | A | S | Y | R | V | F | K | K |
| 3.3 | W | R | H | N | P | | R | N | F | S | V | N | Y | G | A | S | Y | R | V | F | K | K |
| 3.4 | W | R | H | N | P | | R | N | F | S | V | N | Y | G | A | S | Y | R | V | F | K | K |
| 3.5 | W | R | H | N | P | | R | N | F | S | V | N | Y | G | A | S | Y | R | V | F | K | K |
| 3.6 | W | R | H | N | P | | R | N | F | S | V | N | Y | G | A | S | Y | R | V | F | K | K |
| 3.7 | W | R | H | N | P | | R | N | F | S | V | N | Y | G | A | S | Y | R | V | F | K | K |
| 3.8 | W | R | H | N | P | | R | N | F | S | V | N | Y | G | A | S | Y | R | V | F | K | K |
| 4.1 | W | R | H | N | P | | R | N | L | S | V | N | H | G | N | S | Y | R | V | I | K | K |
| 4.2 | W | G | H | N | P | | R | N | L | S | V | N | H | G | N | S | Y | R | V | I | K | K |
| 4.3 | W | R | H | N | P | | R | N | F | S | V | N | Y | G | N | S | Y | R | V | F | K | K |
| 5.1 | W | R | L | N | P | | L | N | F | S | V | N | Y | G | A | C | Y | R | V | F | N | K |
| 5.2 | W | R | H | S | P | | R | N | F | S | V | N | Y | G | A | S | Y | R | V | F | K | T |
| 5.3 | W | R | L | N | P | | L | N | I | S | V | N | Y | G | A | C | Y | R | V | I | N | K |
| 5.4 | W | R | H | S | P | | R | N | F | S | V | N | Y | G | A | S | Y | R | V | F | K | T |
| 5.5 | W | R | L | N | P | | L | N | F | S | V | N | Y | G | A | C | Y | R | V | F | N | K |
| 5.6 | W | R | L | N | P | | L | N | F | S | V | N | Y | G | A | C | Y | R | V | F | N | K |
| 5.7 | W | R | L | N | P | | L | N | F | S | V | N | Y | G | A | C | Y | R | V | F | N | K |
| 5.8 | W | R | L | N | P | | L | N | F | S | V | N | Y | G | A | C | Y | R | V | F | N | K |
| 5.9 | W | R | L | N | P | | L | N | F | S | V | N | Y | G | A | C | Y | R | V | F | N | K |
| 5.10 | W | R | L | N | P | | L | N | F | S | V | N | Y | G | A | C | Y | R | V | F | N | K |
| 5.11 | W | R | L | N | P | | L | N | F | S | V | N | Y | G | A | C | Y | R | V | F | N | K |
| 5.12 | W | R | L | N | P | | L | N | F | S | V | N | Y | G | A | C | Y | R | V | F | N | K |
| 5.13 | W | R | H | N | P | | L | D | F | S | V | N | Y | A | A | S | Y | R | V | F | K | K |
| 5.14 | W | R | H | N | S | | L | N | F | C | V | N | Y | G | A | S | Y | R | V | F | K | K |
| 6.1 | W | R | H | N | S | | L | N | F | S | V | N | Y | G | N | S | Y | R | V | F | K | K |
| 6.2 | W | R | H | N | T | V | L | N | F | S | V | N | Y | G | N | S | Y | R | V | F | N | K |
| 6.3 | W | R | L | N | S | | L | N | F | S | V | N | Y | G | A | C | Y | R | V | F | N | K |
| 6.4 | W | R | L | N | S | | L | N | F | S | V | N | Y | G | N | S | Y | R | V | F | N | K |
| 6.5 | W | R | L | N | I | V | L | N | F | S | V | N | Y | G | A | C | Y | R | V | F | N | K |
| 6.6 | W | R | L | N | T | V | L | N | F | S | V | N | Y | G | N | C | Y | R | V | F | N | K |
| 7.1 | W | R | L | N | A | | L | N | F | S | V | N | Y | G | A | C | Y | R | V | F | N | K |
| 7.2 | W | R | L | N | A | | L | N | F | S | V | N | Y | G | N | C | Y | R | V | F | N | K |
| 7.3 | I | R | L | N | A | | L | N | F | S | V | N | Y | G | A | C | Y | R | V | F | N | K |
| 7.4 | R | R | L | N | A | | L | N | F | S | V | N | Y | G | A | C | Y | R | V | F | N | K |
| 7.5 | W | R | L | N | A | | L | N | F | S | V | N | Y | G | A | C | Y | H | V | F | N | K |
| 7.6 | W | R | L | N | A | | L | N | I | S | V | N | Y | G | A | C | Y | P | V | I | N | K |
| 7.7 | L | R | L | N | A | | L | N | F | S | V | N | Y | G | A | C | Y | P | V | F | N | K |
| 7.8 | I | R | L | N | A | | L | N | F | S | V | N | Y | G | N | C | Y | R | V | F | N | K |
| 7.9 | L | R | L | N | A | | L | N | F | S | V | N | Y | G | N | C | Y | P | V | F | N | K |
| 7.10 | R | R | L | N | A | | L | N | F | S | V | N | Y | G | A | C | Y | P | V | F | N | K |

In some embodiments, the TadA is provided as a monomer or dimer (e.g., a heterodimer of wild-type E. coli TadA and an engineered TadA variant). In some embodiments, the adenosine deaminase is an eighth generation TadA*8 variant as shown in Table 7 below.

TABLE 7
TadA8* Adenosine Deaminase Variants

| Adenosine | |
|---|---|
| Deaminase | Adenosine Deaminase Description |
| TadA*8.1 | Monomer_TadA*7.10 + Y147T |
| TadA*8.2 | Monomer_TadA*7.10 + Y147R |
| TadA*8.3 | Monomer_TadA*7.10 + Q154S |
| TadA*8.4 | Monomer_TadA*7.10 + Y123H |
| TadA*8.5 | Monomer_TadA*7.10 + V82S |
| TadA*8.6 | Monomer_TadA*7.10 + T166R |
| TadA*8.7 | Monomer_TadA*7.10 + Q154R |
| TadA*8.8 | Monomer_TadA*7.10 + Y147R_Q154R_Y123H |
| TadA*8.9 | Monomer_TadA*7.10 + Y147R_Q154R_I76Y |
| TadA*8.10 | Monomer_TadA*7.10 + Y147R_Q154R_T166R |
| TadA*8.11 | Monomer_TadA*7.10 + Y147T_Q154R |
| TadA*8.12 | Monomer_TadA*7.10 + Y147T_Q154S |
| TadA*8.13 | Monomer_TadA*7.10 + H123H_Y147R_Q154R_I76Y |
| TadA*8.14 | Heterodimer_(WT) + (TadA*7.10 + Y147T) |
| TadA*8.15 | Heterodimer_ (WT) + (TadA*7.10 + Y147R) |
| TadA*8.16 | Heterodimer_(WT) + (TadA*7.10 + Q154S) |
| TadA*8.17 | Heterodimer_(WT) + (TadA*7.10 + Y123H) |
| TadA*8.18 | Heterodimer_(WT) + (TadA*7.10 + V82S) |
| TadA*8.19 | Heterodimer_(WT) + (TadA*7.10 + T166R) |
| TadA*8.20 | Heterodimer_(WT) + (TadA*7.10 + Q154R) |
| TadA*8.21 | Heterodimer_(WT) + (TadA*7.10 + |
| | Y147R_Q154R_Y123H) |
| TadA*8.22 | Heterodimer_(WT) + (TadA*7.10 + |
| | Y147R_Q154R_I76Y) |
| TadA*8.23 | Heterodimer_(WT) + (TadA*7.10 + |
| | Y147R_Q154R_T166R) |
| TadA*8.24 | Heterodimer_(WT) + (TadA*7.10 + Y147T_Q154R) |
| TadA*8.25 | Heterodimer_(WT) + (TadA*7.10 + Y147T_Q154S) |
| TadA*8.26 | Heterodimer_(WT) + (TadA*7.10 + |
| | H123H_Y147T_Q154R_I76Y) |

In some embodiments, the adenosine deaminase is a ninth generation TadA*9 variant containing an alteration at an amino acid position selected from the following: 21, 23, 25, 38, 51, 54, 70, 71, 72, 72, 94, 124, 133, 138, 139, 146, and 158 of a TadA variant as shown in the reference sequence below:

```
(SEQ ID NO: 130)
      10 20 30 40 50
MSEVEFSHEY WMRHALTLAK RARDEREVPV GAVLVLNNRV
                            60 70 80 90 100
IGEGWNRAIG LHDPTAHAEI MALRQGGLVM QNYRLIDATL
                                                110 120 130 140
YVTFEPCVMC AGAMIHSRIG RVVFGVRNAK TGAAGSLMDV
    150 160
LHYPGMNHRV EITEGILADE CAALLCYFFR MPRQVFNAQK
KAQSSTD
```

In one embodiment, the adenosine deaminase variant contains alterations at two or more amino acid positions selected from the following: 21, 23, 25, 38, 51, 54, 70, 71, 72, 94, 124, 133, 138, 139, 146, and 158 of the TadA reference sequence above. In another embodiment, the adenosine deaminase variant contains one or more (e.g., 2, 3, 4) alterations selected from the following: R21N, R23H, E25F, N38G, L51W, P54C, M70V, Q71M, N72K, Y73S, M94V, P124W, T133K, D139L, D139M, C146R, and A158K of SEQ ID NO. 1. In other embodiments, the adenosine deaminase variant further contains one or more of the following alterations: Y147T, Y147R, Q154S, Y123H, and Q154R. In still other embodiments, the adenosine deaminase variant contains a combination of alterations relative to the above TadA reference sequence selected from the following: E25F+V82S+Y123H, T133K+Y147R+Q154R; E25F+V82S+Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T+Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R+Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; M70V+V82S+M94V+Y123H+Y147R+Q154R; Q71M+V82S+Y123H+Y147R+Q154R; E25F+176Y+V82S+Y123H+Y147R+Q154R; 176Y+V82T+Y123H+Y147R+Q154R; N38G+176Y+V82S+Y123H+Y147R+Q154R; R23H+176Y+V82S+Y123H+Y147R+Q154R; P54C+176Y+V82S+Y123H+Y147R+Q154R; R21N+I76Y+V82S+Y123H+Y147R+Q154R; I76Y+V82S+Y123H+D138M+Y147R+Q154R; Y72S+176Y+V82S+Y123H+Y147R+Q154R; E25F+176Y+V82S+Y123H+Y147R+Q154R; I76Y+V82T+Y123H+Y147R+Q154R; N38G+176Y+V82S+Y123H+Y147R+Q154R; R23H+176Y+V82S+Y123H+Y147R+Q154R; P54C+176Y+V82S+Y123H+Y147R+Q154R; R21N+176Y+V82S+Y123H+Y147R+Q154R; I76Y+V82S+Y123H+D138M+Y147R+Q154R; Y72S+176Y+V82S+Y123H+Y147R+Q154R; and V82S+Q154R; N72K+V82S+Y123H+Y147R+Q154R; Q71M_V82S+Y123H+Y147R+Q154R; V82S+Y123H+T133K+Y147R+Q154R; V82S+Y123H+T133K+Y147R+Q154R+A158K; M70V+Q71M+N72K+V82S+Y123H+Y147R+Q154R; N72K_V82S+Y123H+Y147R+Q154R; Q71M_V82S+Y123H+Y147R+Q154R; M70V+V82S+M94V+Y123H+Y147R+Q154R; V82S+Y123H+T133K+Y147R+Q154R; V82S+Y123H+T133K+Y147R+Q154R+A158K; and M70V+Q71M+N72K+V82S+Y123H+Y147R+Q154R. In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, the fusion proteins as described herein comprise one or more adenosine deaminase domains. In some embodiments, the adenosine deaminases provided herein are capable of deaminating adenine. In some embodiments, the adenosine deaminases provided herein are capable of deaminating adenine in a deoxyadenosine residue of DNA. The adenosine deaminase may be derived from any suitable organism (e.g., E. coli). In some embodiments, the adenine deaminase is a naturally-occurring adenosine deaminase that includes one or more mutations corresponding to any of the mutations provided herein (e.g., mutations in ecTadA). One of skill in the art will be able to identify the corresponding residue in any homologous protein, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine in RNA. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor. A base editor comprising an adenosine deaminase can act on any polynucleotide, including RNA, DNA, and DNA-RNA hybrids.

In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion of adenosine deaminase acting on RNA (ADAR, e.g., ADAR1 or ADAR2) or tRNA (ADAT). A base editor comprising an adenosine deaminase domain can also be capable of deaminating an A nucleobase of a DNA polynucleotide. In an embodiment an adenosine deaminase domain of a base editor comprises all or a portion of an ADAT comprising one or more mutations which permit the ADAT to deaminate a target A in DNA. For example, the base editor can comprise all or a portion of an ADAT from Escherichia coli (EcTadA) comprising one or more of the following mutations: D108N, A106V, D147Y, E155V, L84F, H123Y, I156F, or a corresponding mutation in another adenosine deaminase.

In some embodiments, a base editor described herein comprises a fusion protein comprising an adenosine deaminase domain (e.g., adenosine deaminase variant domain). In some embodiments, an adenosine deaminase variant domain contains a combination of alterations in a TadA*7.10 amino acid sequence, where the combinations are V82G, Y147T/D, Q154S, and one or more of L36H, 176Y, F149Y, N157K, and D167N. In some embodiments, the combinations of alterations in a TadA*7.10 amino acid sequence are V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; 176Y+V82G+Y147D+F149Y+Q154S+D167N; or L36H+176Y+V82G+Y147D+F149Y+Q154S+N157K+D167N or a corresponding alteration in another adenosine deaminase. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a adenosine residue in RNA.

In some embodiments, the nucleobase editors provided herein can be made by fusing together one or more protein domains, thereby generating a fusion protein. In certain embodiments, the fusion proteins provided herein comprise one or more features that improve the base editing activity (e.g., efficiency, selectivity, and specificity) of the fusion proteins. For example, the fusion proteins provided herein can comprise a Cas13 domain that has reduced nuclease activity. In some embodiments, the fusion proteins provided herein can have a Cas13 domain that does not have nuclease activity, i.e. a deadCas13 or dCas13 or a Cas13 domain that nicks ssRNA or one strand of an RNA: DNA hybrid, i.e. a nickase or nCas9. Without wishing to be bound by any particular theory, the presence of the catalytic residue (e.g., H840) maintains the activity of the Cas13 to cleave the non-edited (e.g., non-deaminated) strand containing a T opposite the targeted A. Mutation of the catalytic residue of Cas13 prevents cleavage of the edited strand containing the targeted A residue. Such Cas13 variants are able to generate a single-strand break (nick) for example in an RNA: DNA hybrid at a specific location based on the gRNA-defined target sequence, leading to repair of the non-edited strand, ultimately resulting in a T to C change on the non-edited strand. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

Provided and described herein are adenosine deaminase variants that have increased efficiency (>50-60%) and specificity. In particular, the adenosine deaminase variants described herein are more likely to edit a desired base within a polynucleotide, and are less likely to edit bases that are not intended to be altered (i.e., “bystanders”).

In some embodiments, the adenosine deaminase is a TadA deaminase. In particular embodiments, the TadA is any one of the TadA described in PCT/US2017/045381 (WO 2018/027078), which is incorporated herein by reference in its entirety.

A wild type TadA (wt) adenosine deaminase has the following sequence (also termed TadA reference sequence):

(SEQ ID NO: 131)
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD

In some embodiments the adenosine deaminase is a full-length E. coli TadA deaminase. For example, in certain embodiments, the adenosine deaminase comprises the amino acid sequence:

(SEQ ID NO: 132)
MRRAFITGVFFLSEVEFSHEYWMRHALTLAKRAWDEREVPVGAVLVHNNR
VIGEGWNRPIGRHDPTAHAEIMALRQGGLVMQNYRLIDATLYVTLEPCVM
CAGAMIHSRIGRVVFGARDAKTGAAGSLMDVLHHPGMNHRVEITEGILAD
ECAALLSDFFRMRRQEIKAQKKAQSSTD.

In some embodiments, the adenosine deaminase is from a prokaryote. In some embodiments, the adenosine deaminase is from a bacterium. In some embodiments, the adenosine deaminase is from Escherichia coli (E. coli), Staphylococcus aureus (S. aureus), Salmonella typhimurium (S. typhimurium), Shewanella putrefaciens (S. putrefaciens), Haemophilus influenzae (H. influenzae), Caulobacter crescentus (C. crescentus), Geobacter sulfurreducens (G. sulfurreducens), or Bacillus subtilis. In some embodiments, the adenosine deaminase is from E. coli.

It should be appreciated, however, that additional adenosine deaminases useful in the present application would be apparent to the skilled artisan and are within the scope of this disclosure. For example, the adenosine deaminase may be a homolog of adenosine deaminase acting on tRNA (ADAT). Without limitation, the amino acid sequences of exemplary AD AT homologs include the following:

Staphylococcus aureus (S. aureus) TadA:
(SEQ ID NO: 133)
MGSHMTNDIYFMTLAIEEAKKAAQLGEVPIGAIITKDDEVIARAHNLRET
LQQPTAHAEHIAIERAAKVLGSWRLEGCTLYVTLEPCVMCAGTIVMSRIP
RVVYGADDPKGGCSGSLMNLLQQSNFNHRAIVDKGVLKEACSTLLTTFFK
NLRANKKSTN
Bacillus subtilis (B. subtilis) TadA:
(SEQ ID NO: 134)
MTQDELYMKEAIKEAKKAEEKGEVPIGAVLVINGEIIARAHNLRETEQRS
IAHAEMLVIDEACKALGTWRLEGATLYVTLEPCPMCAGAVVLSRVEKVVF
GAFDPKGGCSGTLMNLLQEERFNHQAEVVSGVLEEECGGMLSAFFRELRK
KKKAARKNLSE
Salmonella typhimurium (S. typhimurium) TadA:
(SEQ ID NO: 135)
MPPAFITGVTSLSDVELDHEYWMRHALTLAKRAWDEREVPVGAVLVHNHR
VIGEGWNRPIGRHDPTAHAEIMALRQGGLVLQNYRLLDTTLYVTLEPCVM
CAGAMVHSRIGRVVFGARDAKTGAAGSLIDVLHHPGMNHRVEIIEGVLRD
ECATLLSDFFRMRRQEIKALKKADRAEGAGPAV
Shewanella putrefaciens (S. putrefaciens) TadA:
(SEQ ID NO: 136)
MDEYWMQVAMQMAEKAEAAGEVPVGAVLVKDGQQIATGYNLSISQHDPTA
HAEILCLRSAGKKLENYRLLDATLYITLEPCAMCAGAMVHSRIARVVYGA
RDEKTGAAGTVVNLLQHPAFNHQVEVTSGVLAEACSAQLSRFFKRRRDEK
KALKLAQRAQQGIE
Haemophilus influenzae F3031 (H. influenzae) TadA:
(SEQ ID NO: 137)
MDAAKVRSEFDEKMMRYALELADKAEALGEIPVGAVLVDDARNIIGEGWN
LSIVQSDPTAHAEIIALRNGAKNIQNYRLLNSTLYVTLEPCTMCAGAILH
SRIKRLVFGASDYKTGAIGSRFHFFDDYKMNHTLEITSGVLAEECSQKLS
TFFQKRREEKKIEKALLKSLSDK
Caulobacter crescentus (C. crescentus) TadA:
(SEQ ID NO: 138)
MRTDESEDQDHRMMRLALDAARAAAEAGETPVGAVILDPSTGEVIATAGN
GPIAAHDPTAHAEIAAMRAAAAKLGNYRLTDLTLVVTLEPCAMCAGAISH
ARIGRVVFGADDPKGGAVVHGPKFFAQPTCHWRPEVTGGVLADESADLLR
GFFRARRKAKI
Geobacter sulfurreducens (G. sulfurreducens) TadA:
(SEQ ID NO: 139)
MSSLKKTPIRDDAYWMGKAIREAAKAAARDEVPIGAVIVRDGAVIGRGHN
LREGSNDPSAHAEMIAIRQAARRSANWRLTGATLYVTLEPCLMCMGAIIL
ARLERVVFGCYDPKGGAAGSLYDLSADPRLNHQVRLSPGVCQEECGTMLS
DFFRDLRRRKKAKATPALFIDERKVPPEP
An embodiment of E. Coli TadA (ecTadA) includes
the following:
(SEQ ID NO: 140)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

It should be appreciated that any of the mutations provided herein (e.g., based on the TadA reference sequence) can be introduced into other adenosine deaminases, such as E. coli TadA (ecTadA), S. aureus TadA (saTadA), or other adenosine deaminases (e.g., bacterial adenosine deaminases). It would be apparent to the skilled artisan that additional deaminases may similarly be aligned to identify homologous amino acid residues that can be mutated as provided herein. Thus, any of the mutations identified in the TadA reference sequence can be made in other adenosine deaminases (e.g., ecTada) that have homologous amino acid residues. It should also be appreciated that any of the mutations provided herein can be made individually or in any combination in the TadA reference sequence or another adenosine deaminase.

In some embodiments, the adenosine deaminase comprises a D108X mutation in the TadA reference sequence, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in TadA reference sequence, or a corresponding mutation in another adenosine deaminase. It should be appreciated, however, that additional deaminases may similarly be aligned to identify homologous amino acid residues that can be mutated as provided herein.

In some embodiments, the adenosine deaminase comprises an A106X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A106V mutation in TadA reference sequence, or a corresponding mutation in another adenosine deaminase (e.g., ecTadA).

In some embodiments, the adenosine deaminase comprises a E155X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in TadA reference sequence, or a corresponding mutation in another adenosine deaminase (e.g., ecTadA).

In some embodiments, the adenosine deaminase comprises a D147X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a D147Y, mutation in TadA reference sequence, or a corresponding mutation in another adenosine deaminase (e.g., ecTadA).

In some embodiments, the adenosine deaminase comprises an A106X, E155X, or D147X, mutation in the TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an E155D, E155G, or E155V mutation. In some embodiments, the adenosine deaminase comprises a D147Y.

It should be appreciated that any of the mutations provided herein (e.g., based on the ecTadA amino acid sequence of TadA reference sequence) may be introduced into other adenosine deaminases, such as S. aureus TadA (saTadA), or other adenosine deaminases (e.g., bacterial adenosine deaminases). It would be apparent to the skilled artisan how to are homologous to the mutated residues in ecTadA. Thus, any of the mutations identified in ecTadA may be made in other adenosine deaminases that have homologous amino acid residues. It should also be appreciated that any of the mutations provided herein may be made individually or in any combination in ecTadA or another adenosine deaminase.

For example, an adenosine deaminase contains a combination of mutations (e.g., V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; I76Y+V82G+Y147D+F149Y+Q154S+D167N; or L36H+I76Y+V82G+Y147D+F149Y+Q154S+N157K+D167N), and may contain one or more additional mutations. Additional mutations include, for example, a D108N, a A106V, a E155V, and/or a D147Y mutation in TadA reference sequence, or a corresponding mutation in another adenosine deaminase (e.g., ecTadA). In some embodiments, an adenosine deaminase comprises the following group of mutations (groups of mutations are separated by a “;”) in TadA reference sequence, or corresponding mutations in another adenosine deaminase: D108N and A106V; D108N and E155V; D108N and D147Y; A106V and E155V; A106V and D147Y; E155V and D147Y; D108N, A106V, and E155V; D108N, A106V, and D147Y; D108N, E155V, and D147Y; A106V, E155V, and D147Y; and D108N, A106V, E155V, and D147Y. It should be appreciated, however, that any combination of corresponding mutations provided herein may be made in an adenosine deaminase (e.g., ecTadA).

In some embodiments, the adenosine deaminase comprises one or more of a H8X, T17X, L18X, W23X, L34X, W45X, R51X, A56X, E59X, E85X, M94X, 195X, V102X, F104X, A106X, R107X, D108X, K110X, M118X, N127X, A138X, F149X, M151X, R153X, Q154X, I156X, and/or K157X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, I95L, V102A, F104L, A106V, R107C, or R107H, or R107P, D108G, or D108N, or D108V, or D108A, or D108Y, K110I, M118K, N127S, A138V, F149Y, M151V, R153C, Q154L, I156D, and/or K157R mutation in TadA reference sequence, or one or more corresponding mutations in another adenosine deaminase.

In some embodiments, the adenosine deaminase comprises one or more of a H8X, D108X, and/or N127X mutation in TadA reference sequence, or one or more corresponding mutations in another adenosine deaminase, where X indicates the presence of any amino acid. In some embodiments, the adenosine deaminase comprises one or more of a H8Y, D108N, and/or N127S mutation in TadA reference sequence, or one or more corresponding mutations in another adenosine deaminase.

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in TadA reference sequence, or one or more corresponding mutations in another adenosine deaminase, where X indicates the presence of any amino acid other than the corresponding amino acid in the wild-type adenosine deaminase. In some embodiments, the adenosine deaminase comprises one or more of H8Y, R26W, M61I, L68Q, M70V, A106T, D108N, A109T, N127S, D147Y, R152C, Q154H or Q154R, E155G or E155V or E155D, K161Q, Q163H, and/or T166P mutation in TadA reference sequence, or one or more corresponding mutations in another adenosine deaminase.

In some embodiments, the adenosine deaminase comprises one, two, three, four, five, or six mutations selected from the group consisting of H8X, D108X, N127X, D147X, R152X, and Q154X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in TadA reference sequence, or a corresponding mutation or mutations in another adenosine deaminase (e.g., ecTadA), where X indicates the presence of any amino acid other than the corresponding amino acid in the wild-type adenosine deaminase.

In some embodiments, the adenosine deaminase comprises one, two, three, four, five, or six mutations selected from the group consisting of H8X, A106X, and D108X, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, R26X, L68X, D108X, N127X, D147X, and E155X, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of H8X, R126X, L68X, D108X, N127X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, D108X, A109X, N127X, and E155X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, M61I, M70V, D108N, N127S, Q154R, E155G and Q163H in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, N127S, E155V, and T166P in TadA reference sequence, or a corresponding mutation or mutations in another adenosine deaminase (e.g., ecTadA). In some embodiments, the adenosine deaminase comprises one, two, three, four, five, or six mutations selected from the group consisting of H8Y, A106T, D108N, N127S, E155D, and K161Q in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, R26W, L68Q, D108N, N127S, D147Y, and E155V in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, A109T, N127S, and E155G in TadA reference sequence, or a corresponding mutation or mutations in another adenosine deaminase (e.g., ecTadA).

In some embodiments, the adenosine deaminase comprises one or more of the or one or more corresponding mutations in another adenosine deaminase. In some embodiments, the adenosine deaminase comprises a D108N, D108G, or D108V mutation in TadA reference sequence, or corresponding mutations in another adenosine deaminase. In some embodiments, the adenosine deaminase comprises a A106V and D108N mutation in TadA reference sequence, or corresponding mutations in another adenosine deaminase. In some embodiments, the adenosine deaminase comprises R107C and D108N mutations in TadA reference sequence, or corresponding mutations in another adenosine deaminase. In some embodiments, the adenosine deaminase comprises a H8Y, D108N, N127S, D147Y, and Q154H mutation in TadA reference sequence, or corresponding mutations in another adenosine deaminase. In some embodiments, the adenosine deaminase comprises a H8Y, D108N, N127S, D147Y, and E155V mutation in TadA reference sequence, or corresponding mutations in another adenosine deaminase. In some embodiments, the adenosine deaminase comprises a D108N, D147Y, and E155V mutation in TadA reference sequence, or corresponding mutations in another adenosine deaminase. In some embodiments, the adenosine deaminase comprises a H8Y, D108N, and N127S mutation in TadA reference sequence, or corresponding mutations in another adenosine deaminase. In some embodiments, the adenosine deaminase comprises a A106V, D108N, D147Y, and E155V mutation in TadA reference sequence, or corresponding mutations in another adenosine deaminase (e.g., ecTadA).

In some embodiments, the adenosine deaminase comprises one or more of S2X, H8X, 149X, L84X, H123X, N127X, I156X, and/or K160X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, 149F, L84F, H123Y, N127S, I156F, and/or K160S mutation in TadA reference sequence, or one or more corresponding mutations in another adenosine deaminase (e.g., ecTadA).

In some embodiments, the adenosine deaminase comprises an L84X mutation adenosine deaminase, where X indicates any amino acid other than the corresponding amino acid in the wild-type adenosine deaminase. In some embodiments, the adenosine deaminase comprises an L84F mutation in TadA reference sequence, or a corresponding mutation in another adenosine deaminase (e.g., ecTadA).

In some embodiments, the adenosine deaminase comprises an H123X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H123Y mutation in TadA reference sequence, or a corresponding mutation in another adenosine deaminase.

In some embodiments, the adenosine deaminase comprises an I156X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an I156F mutation in TadA reference sequence, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and I156X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of S2X, 149X, A106X, D108X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, A106X, D108X, N127X, and K160X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in TadA reference sequence, or a corresponding mutation or mutations in another adenosine deaminase. In some embodiments, the adenosine deaminase comprises one, two, three, four, five, or six mutations selected from the group consisting of S2A, 149F, A106V, D108N, D147Y, and E155V in TadA reference sequence.

In some embodiments, the adenosine deaminase comprises one, two, three, four, or five mutations selected from the group consisting of H8Y, A106T, D108N, N127S, and K160S in TadA reference sequence, or a corresponding mutation or mutations in another adenosine deaminase.

In some embodiments, the adenosine deaminase comprises one or more of a E25X, R26X, R107X, A142X, and/or A143X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R107K, R107A, R107N, R107W, R107H, R107S, A142N, A142D, A142G, A143D, A143G, A143E, A143L, A143W, A143M, A143S, A143Q, and/or A143R mutation in TadA reference sequence, or one or more corresponding mutations in another adenosine deaminase. In some embodiments, the adenosine deaminase comprises one or more of the mutations described herein corresponding to TadA reference sequence, or one or more corresponding mutations in another adenosine deaminase.

In some embodiments, the adenosine deaminase comprises an E25X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an E25M, E25D, E25A, E25R, E25V, E25S, or E25Y mutation in TadA reference sequence, or a corresponding mutation in another adenosine deaminase (e.g., ecTadA).

In some embodiments, the adenosine deaminase comprises an R26X mutation in TadA reference sequence, or a corresponding mutation in another adenosine deaminase, where X indicates any amino acid other than the corresponding amino acid in the wild-type adenosine deaminase. In some embodiments, the adenosine deaminase comprises R26G, R26N, R26Q, R26C, R26L, or R26K mutation in TadA reference sequence, or a corresponding mutation in another adenosine deaminase (e.g., ecTadA).

In some embodiments, the adenosine deaminase comprises an R107X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R107P, R107K, R107A, R107N, R107W, R107H, or R107S mutation in TadA reference sequence, or a corresponding mutation in another adenosine deaminase (e.g., ecTadA).

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in TadA reference sequence, or a corresponding mutation in another adenosine deaminase (e.g., ecTadA).

In some embodiments, the adenosine deaminase comprises an A143X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A143D, A143G, A143E, A143L, A143W, A143M, A143S, A143Q, and/or A143R mutation in TadA reference sequence, or a corresponding mutation in another adenosine deaminase (e.g., ecTadA).

In some embodiments, the adenosine deaminase comprises one or more of a H36X, N37X, P48X, 149X, R51X, M70X, N72X, D77X, E134X, S146X, Q154X, K157X, and/or K161X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H36L, N37T, N37S, P48T, P48L, I49V, R51H, R51L, M70L, N72S, D77G, E134G, S146R, S146C, Q154H, K157N, and/or K161T mutation in TadA reference sequence, or one or more corresponding mutations in another adenosine deaminase (e.g., ecTadA).

In some embodiments, the adenosine deaminase comprises an H36X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H36L mutation in TadA reference sequence, or a corresponding mutation in another adenosine deaminase.

In some embodiments, the adenosine deaminase comprises an N37X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an N37T or N37S mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an P48T or P48L mutation in TadA reference sequence, or a corresponding mutation in another adenosine deaminase.

In some embodiments, the adenosine deaminase comprises an R51X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R51H or R51L mutation in TadA reference sequence, or a corresponding mutation in another adenosine deaminase.

In some embodiments, the adenosine deaminase comprises an S146X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an S146R or S146C mutation in TadA reference sequence, or a corresponding mutation in another adenosine deaminase.

In some embodiments, the adenosine deaminase comprises an K157X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a K157N mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a P48S, P48T, or P48A mutation in TadA reference sequence, or a corresponding mutation in another adenosine deaminase.

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a A142N mutation in TadA reference sequence, or a corresponding mutation in another adenosine deaminase.

In some embodiments, the adenosine deaminase comprises an W23X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a W23R or W23L mutation in TadA reference sequence, or a corresponding mutation in another adenosine deaminase.

In some embodiments, the adenosine deaminase comprises an R152X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a R152P or R52H mutation in TadA reference sequence, or a corresponding mutation in another adenosine deaminase.

In one embodiment, the adenosine deaminase may comprise the mutations H36L, R51L, L84F, A106V, D108N, H123Y, S146C, D147Y, E155V, I156F, and K157N. In some embodiments, the adenosine deaminase comprises the following combination of mutations relative to TadA reference sequence, where each mutation of a combination is separated by a “_” and each combination of mutations is between parentheses:
(A106V_D108N),
(R107C_D108N),
(H8Y_D108N_N127S_D147Y_Q154H),
(H8Y_D108N_N127S_D147Y_E155V),
(D108N_D147Y_E155V),
(H8Y_D108N_N127S),
(H8Y_D108N_N127S_D147Y_Q154H),
(A106V_D108N_D147Y_E155V),
(D108Q_D147Y_E155V),
(D108M_D147Y_E155V),
(D108L_D147Y_E155V),
(D108K_D147Y_E155V),
(D108I_D147Y_E155V),
(D108F_D147Y_E155V),
(A106V_D108N_D147Y),
(A106V_D108M_D147Y_E155V),
(E59A_A106V_D108N_D147Y_E155V),
(E59A cat dead_A106V_D108N_D147Y_E155V),
(L84F_A106V_D108N_H123Y_D147Y_E155V_I156Y),
(L84F_A106V_D108N_H123Y_D147Y_E155V_I156F),
(D103A_D104N),
(G22P_D103A_D104N),
(D103A_D104N_S138A),
(R26G_L84F_A106V_R107H_D108N_H123Y_A142N_A143D_D147Y_E155V_I156F),
(E25G_R26G_L84F_A106V_R107H_D108N_H123Y_A142N_A143D_D147Y_E155V_I15 6F),
(E25D_R26G_L84F_A106V_R107K_D108N_H123Y_A142N_A143G_D147Y_E155V_I15 6F), (R26Q_L84F_A106V_D108N_H123Y_A142N_D147Y_E155V_I156F),
(E25M_R26G_L84F_A106V_R107P_D108N_H123Y_A142N_A143D_D147Y_E155V_I15
6F), (R26C_L84F_A106V_R107H_D108N_H123Y_A142N_D147Y_E155V_I156F),
(L84F_A106V_D108N_H123Y_A142N_A143L_D147Y_E155V_I156F),
(R26G_L84F_A106V_D108N_H123Y_A142N_D147Y_E155V_I156F),
(E25A_R26G_L84F_A106V_R107N_D108N_H123Y_A142N_A143E_D147Y_E155V_I15
6F),
(R26G_L84F_A106V_R107H_D108N_H123Y_A142N_A143D_D147Y_E155V_I156F),
(A106V_D108N_A142N_D147Y_E155V),
(R26G_A106V_D108N_A142N_D147Y_E155V),
(E25D_R26G_A106V_R107K_D108N_A142N_A143G_D147Y_E155V),
(R26G_A106V_D108N_R107H_A142N_A143D_D147Y_E155V),
(E25D_R26G_A106V_D108N_A142N_D147Y_E155V),
(A106V_R107K_D108N_A142N_D147Y_E155V),
(A106V_D108N_A142N_A143G_D147Y_E155V),
(A106V_D108N_A142N_A143L_D147Y_E155V),
(H36L_R51L_L84F_A106V_D108N_H123Y_S146C_D147Y_E155V_I156F_K157N),
(N37T_P48T_M70L_L84F_A106V_D108N_H123Y_D147Y_149V_E155V_I156F),
(N37S_L84F_A106V_D108N_H123Y_D147Y_E155V_I156F_K161T),
(H36L_L84F_A106V_D108N_H123Y_D147Y_Q154H_E155V_I156F),
(N72S_L84F_A106V_D108N_H123Y_S146R_D147Y_E155V_I156F),
(H36L_P48L_L84F_A106V_D108N_H123Y_E134G_D147Y_E155V_I156F),
(H36L_L84F_A106V_D108N_H123Y_D147Y_E155V_I156F_K157N)
(H36L_L84F_A106V_D108N_H123Y_S146C_D147Y_E155V_I156F),
(L84F_A106V_D108N_H123Y_S146R_D147Y_E155V_I156F_K161T),
(N37S_R51H_D77G_L84F_A106V_D108N_H123Y_D147Y_E155V_I156F),
(R51L_L84F_A106V_D108N_H123Y_D147Y_E155V_I156F_K157N),
(D24G_Q71R_L84F_H96L_A106V_D108N_H123Y_D147Y_E155V_I156F_K160E),
(H36L_G67V_L84F_A106V_D108N_H123Y_S146T_D147Y_E155V_I156F),
(Q71L_L84F_A106V_D108N_H123Y_L137M_A143E_D147Y_E155V_I156F),
(E25G_L84F_A106V_D108N_H123Y_D147Y_E155V_I156F_Q159L),
(L84F_A91T_F104I_A106V_D108N_H123Y_D147Y_E155V_I156F),
(N72D_L84F_A106V_D108N_H123Y_G125A_D147Y_E155V_I156F),
(P48S_L84F_S97C_A106V_D108N_H123Y_D147Y_E155V_I156F),
(W23G_L84F_A106V_D108N_H123Y_D147Y_E155V_I156F),
(D24G_P48L_Q71R_L84F_A106V_D108N_H123Y_D147Y_E155V_I156F_Q159L),
(L84F_A106V_D108N_H123Y_A142N_D147Y_E155V_I156F),
(H36L_R51L_L84F_A106V_D108N_H123Y_A142N_S146C_D147Y_E155V_I156F_K157 N),
(N37S_L84F_A106V_D108N_H123Y_A142N_D147Y_E155V_I156F_K161T),
(L84F_A106V_D108N_D147Y_E155V_I156F),
(R51L_L84F_A106V_D108N_H123Y_S146C_D147Y_E155V_I156F_K157N_K161T),
(L84F_A106V_D108N_H123Y_S146C_D147Y_E155V_I156F_K161T),
(L84F_A106V_D108N_H123Y_S146C_D147Y_E155V_I156F_K157N_K160E_K161T),
(L84F_A106V_D108N_H123Y_S146C_D147Y_E155V_I156F_K157N_K160E),
(R74Q_L84F_A106V_D108N_H123Y_D147Y_E155V_I156F),
(R74A_L84F_A106V_D108N_H123Y_D147Y_E155V_I156F),
(L84F_A106V_D108N_H123Y_D147Y_E155V_I156F),
(R74Q_L84F_A106V_D108N_H123Y_D147Y_E155V_I156F),
(L84F_R98Q_A106V_D108N_H123Y_D147Y_E155V_I156F),
(L84F_A106V_D108N_H123Y_R129Q_D147Y_E155V_I156F),
(P48S_L84F_A106V_D108N_H123Y_A142N_D147Y_E155V_I156F),
(P48S_A142N),
(P48T_149V_L84F_A106V_D108N_H123Y_A142N_D147Y_E155V_I156F_L157N),
(P48T_I49V_A142N),
(H36L_P48S_R51L_L84F_A106V_D108N_H123Y_S146C_D147Y_E155V_I156F_K157N),
(H36L_P48S_R51L_L84F_A106V_D108N_H123Y_S146C_A142N_D147Y_E155V_I156F),
(H36L_P48T_149V_R51L_L84F_A106V_D108N_H123Y_S146C_D147Y_E155V_156F_K157N),
(H36L_P48T_149V_R51L_L84F_A106V_D108N_H123Y_A142N_S146C_D147Y_E155V_I156F_K157N),
(H36L_P48A_R51L_L84F_A106V_D108N_H123Y_S146C_D147Y_E155V_I156F_K157N),
(H36L_P48A_R51L_L84F_A106V_D108N_H123Y_A142N_S146C_D147Y_E155V_I156F_K157N),
(H36L_P48A_R51L_L84F_A106V_D108N_H123Y_S146C_A142N_D147Y_E155V_I156F_K157N),
(W23L_H36L_P48A_R51L_L84F_A106V_D108N_H123Y_S146C_D147Y_E155V_I156F_K157N),
(W23R_H36L_P48A_R51L_L84F_A106V_D108N_H123Y_S146C_D147Y_E155V_I156F_K157N),
(W23L_H36L_P48A_R51L_L84F_A106V_D108N_H123Y_S146R_D147Y_E155V_I156F_K161T),
(H36L_P48A_R51L_L84F_A106V_D108N_H123Y_S146C_D147Y_R152H_E155V_I156F_K157N),
(H36L_P48A_R51L_L84F_A106V_D108N_H123Y_S146C_D147Y_R152P_E155V_I156F_K157N),
(W23L_H36L_P48A_R51L_L84F_A106V_D108N_H123Y_S146C_D147Y_R152P_E155V_I156F_K157N),
(W23L_H36L_P48A_R51L_L84F_A106V_D108N_H123Y_A142A_S146C_D147Y_E155 V_I156F_K157N),
(W23L_H36L_P48A_R51L_L84F_A106V_D108N_H123Y_A142A_S146C_D147Y_R152P_E155V_I156F_K157N),
(W23L_H36L_P48A_R51L_L84F_A106V_D108N_H123Y_S146R_D147Y_E155V_I156F_K161T),
(W23R_H36L_P48A_R51L_L84F_A106V_D108N_H123Y_S146C_D147Y_R152P_E155V_I156F_K157N),
(H36L_P48A_R51L_L84F_A106V_D108N_H123Y_A142N_S146C_D147Y_R152P_E155V_I156F_K157N).

In some embodiments, the TadA deaminase is TadA variant. In some embodiments, the TadA variant is TadA*7.10. In particular embodiments, the fusion proteins comprise a single TadA*7.10 domain (e.g., provided as a monomer). In other embodiments, the fusion protein comprises TadA*7.10 and TadA (wt), which are capable of forming heterodimers. In one embodiment, a fusion protein as described herein comprises a wild-type TadA linked to TadA*7.10, which is linked to Cas9 nickase.

In some embodiments, TadA*7.10 comprises at least one alteration. In some embodiments, the adenosine deaminase comprises an alteration in the following sequence:

(SEQ ID NO: 141)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD

In some embodiments, TadA*7.10 comprises an alteration at amino acid 82 and/or 166. In particular embodiments, TadA*7.10 comprises one or more of the following alterations: Y147T, Y147R, Q154S, Y123H, V82S, T166R, and/or Q154R. In other embodiments, a variant of TadA*7.10 comprises a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+176Y; V82S+Y123H+Y147R+Q154R; and I76Y+V82S+Y123H+Y147R+Q154R.

In some embodiments, a variant of TadA*7.10 comprises one or more of alterations selected from the group of L36H, 176Y, V82G, Y147T, Y147D, F149Y, Q154S, N157K, and/or D167N. In some embodiments, a variant of TadA*7.10 comprises V82G, Y147T/D, Q154S, and one or more of L36H, 176Y, F149Y, N157K, and D167N. In other embodiments, a variant of TadA*7.10 comprises a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; I76Y+V82G+Y147D+F149Y+Q154S+D167N; L36H+176Y+V82G+Y147D+F149Y+Q154S+N157K+D167N.

In some embodiments, an adenosine deaminase variant (e.g., TadA variant) comprises a deletion. In some embodiments, an adenosine deaminase variant comprises a deletion of the C terminus. In particular embodiments, an adenosine deaminase variant comprises a deletion of the C terminus beginning at residue 149, 150, 151, 152, 153, 154, 155, 156, and 157, relative to TadA*7.10, the TadA reference sequence, or a corresponding mutation in another TadA.

In other embodiments, an adenosine deaminase variant (e.g., TadA*8) is a monomer comprising one or more of the following alterations: Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant (TadA*8) is a monomer comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+176Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant (TadA*8) monomer comprises a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA *7.10, the TadA reference sequence, or a corresponding mutation in another TadA.

In some embodiments, an adenosine deaminase variant (e.g., MSP828) is a monomer comprising one or more of the following alterations L36H, 176Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant (e.g., MSP828) is a monomer comprising V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant (TadA variant) is a monomer comprising a combination of alterations selected from the group of: V82G+Y147T+Q154S; 176Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; I76Y+V82G+Y147D+F149Y+Q154S+D167N; L36H+176Y+V82G+Y147D+F149Y+Q154S+N157K+D167N, relative to TadA*7.10, the TadA reference sequence, or a corresponding mutation in another TadA.

In other embodiments, the adenosine deaminase variant is a homodimer comprising two adenosine deaminase domains (e.g., TadA*8) each having one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+176Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor of the disclosure comprising an adenosine deaminase variant (e.g., TadA*8) homodimer comprising two adenosine deaminase domains (e.g., TadA*8) each having one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In some embodiments, an adenosine deaminase variant is a homodimer comprising two adenosine deaminase domains (e.g., TadA*7.10) each having one or more of the following alterations L36H, 176Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a homodimer comprising two adenosine deaminase variant domains (e.g., MSP828) each having the following alterations V82G, Y147T/D, Q154S, and one or more of L36H, 176Y, F149Y, N157K, and D167N, relative to TadA*7.10, the TadA reference sequence, or a corresponding mutation in another TadA. In other embodiments, the adenosine deaminase variant is a homodimer comprising two adenosine deaminase domains (e.g., TadA*7.10) each having a combination of alterations selected from the group of: V82G+Y147T+Q154S; 176Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; I76Y+V82G+Y147D+F149Y+Q154S+D167N; L36H+176Y+V82G+Y147D+F149Y+Q154S+N157K+D167N, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+176Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*7.10) comprising one or more of the following alterations L36H, 176Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a heterodimer comprising a wild-type adenosine deaminase domain and an adenosine deaminase variant domain (e.g., MSP828) having the following alterations V82G, Y147T/D, Q154S, and one or more of L36H, 176Y, F149Y, N157K, and D167N, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*7.10) comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+176Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor comprises a heterodimer of a TadA*7.10 domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a TadA*7.10 domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*7.10) comprising one or more of the following alterations L36H, 176Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a heterodimer comprising a TadA*7.10 domain and an adenosine deaminase variant domain (e.g., MSP828) having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*7.10) comprising a combination of alterations selected from the group of: V82G+Y147T+Q154S; 176Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; I76Y+V82G+Y147D+F149Y+Q154S+D167N; L36H+176Y+V82G+Y147D+F149Y+Q154S+N157K+D167N, relative to TadA*7.10, the TadA reference sequence, or a corresponding mutation in another TadA.

In some embodiments, the TadA is a variant as shown in Tables 6-9. The tables show certain amino acid position numbers in the TadA amino acid sequence and the amino acids present in those positions in the TadA-7.10 adenosine deaminase. The tables also show amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e.

In particular embodiments, an adenosine deaminase heterodimer can comprise a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, an adenosine deaminase is a TadA*8. In one embodiment, an adenosine deaminase is a TadA*8 that comprises or consists essentially of the following sequence or a fragment thereof having adenosine deaminase activity:

(SEQ ID NO: 142)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCTFFR
MPRQVFNAQKKAQSSTD

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In one embodiment, a fusion protein as described and/or exemplified herein comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the base editor comprises TadA*8 and TadA (wt), which are capable of forming heterodimers.

In some embodiments the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

TABLE 8
Additional TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 9. Table 9 shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 9
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In one embodiment, a fusion protein as described herein comprises a wild-type TadA is linked to an adenosine deaminase variant described herein, which is linked to Cas9 nickase. In particular embodiments, the fusion proteins comprise a single variant TadA domain (e.g., provided as a monomer). In other embodiments, the fusion protein comprises a variant TadA and TadA (wt), which are capable of forming heterodimers.

In some embodiments, the TadA variant is truncated. In some embodiments, the truncated TadA is missing 1, 2, 3, 4, 5, 6, 7, 8, 9, 10, 11, 12, 13, 14, 15, 6, 17, 18, 19, or 20 N-terminal amino acid residues relative to the full length TadA variant. In some embodiments, the truncated TadA variant is missing 1, 2, 3, 4, 5, 6, 7, 8, 9, 10, 11, 12, 13, 14, 15, 6, 17, 18, 19, or 20 C-terminal amino acid residues relative to the full length TadA variant. In some embodiments the adenosine deaminase variant is a full-length TadA variant.

In particular embodiments, the TadA*8 comprises alterations at amino acid position 82 and/or 166 (e.g., V82S, T166R) alone or in combination with any one or more of the following Y147T, Y147R, Q154S, Y123H, and/or Q154R, relative to TadA*7.10, the TadA reference sequence, or a corresponding mutation in another TadA.

In particular embodiments, a combination of alterations is selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+176Y; V82S+Y123H+Y147R+Q154R; and I76Y+V82S+Y123H+Y147R+Q154R, relative to TadA*7.10, the TadA reference sequence, or a corresponding mutation in another TadA. In some embodiments, an adenosine deaminase comprises one or more of the following alterations: R21N, R23H, E25F, N38G, L51W, P54C, M70V, Q71M, N72K, Y73S, V82T, M94V, P124W, T133K, D139L, D139M, C146R, and A158K.

In some embodiments, an adenosine deaminase comprises one or more of the following combinations of alterations: V82S+Q154R+Y147R; V82S+Q154R+Y123H; V82S+Q154R+Y147R+Y123H; Q154R+Y147R+Y123H+176Y+V82S; V82S+I76Y; V82S+Y147R; V82S+Y147R+Y123H; V82S+Q154R+Y123H; Q154R+Y147R+Y123H+176Y; V82S+Y147R; V82S+Y147R+Y123H; V82S+Q154R+Y123H; V82S+Q154R+Y147R; V82S+Q154R+Y147R; Q154R+Y147R+Y123H+I76Y; Q154R+Y147R+Y123H+176Y+V82S; I76Y_V82S_Y123H_Y147R_Q154R; Y147R+Q154R+H123H; and V82S+Q154R.

In some embodiments, an adenosine deaminase comprises one or more of the following combinations of alterations: E25F+V82S+Y123H, T133K+Y147R+Q154R; E25F+V82S+Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T+Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R+Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; and M70V+V82S+M94V+Y123H+Y147R+Q154R

In some embodiments, the TadA*9 variant is a monomer. In some embodiments, the TadA*9 variant is a heterodimer with a wild-type TadA adenosine deaminase. In some embodiments, the TadA*9 variant is a heterodimer with another TadA variant (e.g., TadA*8, TadA*9). Additional details of TadA*9 adenosine deaminases are described in International PCT Application No. PCT/2020/049975, which is incorporated herein by reference for its entirety. In one embodiment, a fusion protein as described herein comprises a wild-type TadA is linked to an adenosine deaminase variant described herein (e.g., TadA variant), which is linked to Cas9 nickase. In particular embodiments, the fusion proteins comprise a single TadA variant domain (e.g., provided as a monomer). In other embodiments, the base editor comprises TadA*8 and TadA (wt), which are capable of forming heterodimers.

In particular embodiments, the fusion proteins comprise a single (e.g., provided as a monomer) TadA variant domain. In some embodiments, the TadA variant is linked to a Cas13 nickase. In some embodiments, the fusion proteins described herein comprise as a heterodimer of a wild-type TadA (TadA (wt)) linked to a TadA variant. In other embodiments, the fusion proteins described herein comprise as a heterodimer of a TadA*7.10 linked to a TadA variant. In some embodiments, the fusion protein comprises a TadA variant monomer. In some embodiments, the fusion protein comprises a heterodimer of a TadA variant and a TadA (wt). In some embodiments, the fusion protein comprises a heterodimer of a TadA variant and TadA*7.10. In some embodiments, the fusion protein comprises a heterodimer of two TadA variants.

In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in TadA reference sequence or another adenosine deaminase (e.g., ecTadA).

Details of A to G nucleobase editing proteins are described in International PCT Application No. PCT/2017/045381 (WO2018/027078) and Gaudelli, N. M., et al., “Programmable base editing of A⋅T to G⋅C in genomic DNA without DNA cleavage” Nature, 551, 464-471 (2017), the entire contents of which are hereby incorporated by reference.

In some embodiments, Cas13 is fused to nuclear localization sequences, including an NLS of the SV40 large T antigen, nucleoplasmin, c-myc, hRNPA1 M9, IBB domain from importin-alpha, NLS of myoma T protein, human p53, c-abl IV, influenza virus NS1, hepatitis virus delta antigen, mouse Mx1, human poly(ADP-ribose) polymerase, steroid hormone receptor (human) glucocorticoid.

In some embodiments, a Cas13 protein is fused to epitope tags including, but not limited to hemagglutinin (HA) tags, histidine (His) tags, FLAG tags, Myc tags, V5 tags, VSV-G tags, SNAP tags, thioredoxin (Trx) tags.

In some embodiments, Cas13 is fused to reporter genes including, but not limited to glutathione-S-transferase (GST), horseradish peroxidase (HRP), chloramphenicol transferase (CAT), HcRed, DsRed, cyan fluorescent protein, yellow fluorescent protein and blue fluorescent protein, green fluorescent protein (GFP), including enhanced versions or superfolded GFP, as well as other modified versions of reporter genes.

In some embodiments, serum half-life of an engineered Cas13 protein is increased by fusion with heterologous proteins such as a human serum albumin protein, transferrin protein, human IgG and/or sialylated peptide, such as the carboxy-terminal peptide (CTP, of chorionic gonadotropin β chain).

In some embodiments, serum half-life of an engineered Cas13 protein is decreased by fusion with destabilizing domains, including but not limited to geminin, ubiquitin, FKBP12-L106P, and/or dihydrofolate reductase.

Suitable fusion partners that provide for increased or decreased stability include, but are not limited to degron sequences. Degrons are readily understood by one of ordinary skill in the art to be amino acid sequences that control the stability of the protein of which they are part. For example, the stability of a protein comprising a degron sequence is controlled at least in part by the degron sequence. In some cases, a suitable degron is constitutive such that the degron exerts its influence on protein stability independent of experimental control (i.e., the degron is not drug inducible, temperature inducible, etc.) In some cases, the degron provides the variant Cas13 polypeptide with controllable stability such that the variant Cas13 polypeptide can be turned “on” (i.e., stable) or “off” (i.e., unstable, degraded) depending on the desired conditions. For example, if the degron is a temperature sensitive degron, the variant Cas13 polypeptide may be functional (i.e., “on”, stable) below a threshold temperature (e.g., 42° C., 41° C., 40° C., 39° C., 38° C., 37° C., 36° C., 35° C., 34° C., 33° C., 32° C., 31° C., 30° C., etc.) but non-functional (i.e., “off, degraded) above the threshold temperature. As another example, if the degron is a drug inducible degron, the presence or absence of drug can switch the protein from an “off (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-1, DHFR, auxins, and/or temperature. Non-limiting examples of suitable degrons are known in the art (e.g., Dohmen et al., Science, 1994. 263 (5151): p. 1273-1276: Heat-inducible degron: a method for constructing temperature-sensitive mutants; Schoeber et al., Am J Physiol Renal Physiol. 2009 January; 296 (1): F204-11: Conditional fast expression and function of multimeric TRPV5 channels using Shield-1; Chu et al., Bioorg Med Chem Lett. 2008 Nov. 15; 18 (22): 5941-4: Recent progress with FKBP-derived destabilizing domains; Kanemaki, Pflugers Arch. 2012 Dec. 28: Frontiers of protein expression control with conditional degrons; Yang et al., Mol Cell. 2012 Nov. 30; 48 (4): 487-8: Titivated for destruction: the methyl degron; Barbour et al., Biosci Rep. 2013 Jan. 18; 33 (1): Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

Exemplary degron sequences have been well-characterized and tested in both cells and animals. Thus, fusing dead Cas13 to a degron sequence produces a “tunable” and “inducible” dead Cas13 polypeptide.

Any of the fusion partners described herein can be used in any desirable combination. As one non-limiting example to illustrate this point, a Cas13 fusion protein can comprise a YFP sequence for detection, a degron sequence for stability, and transcription activator sequence to increase transcription. Furthermore, the number of fusion partners that can be used in a dCas13 fusion protein is unlimited. In some cases, a Cas13 fusion protein comprises one or more (e.g. two or more, three or more, four or more, or five or more) heterologous sequences.

### Target Nucleic Acids

A target nucleic acid is a RNA molecule, which is single-, double-, or multi-stranded RNA, messenger RNA, circular RNA, DNA-RNA hybrids, or a polymer comprising purine and pyrimidine bases or other natural, chemically or biochemically modified, non-natural, or derivatized or modified nucleotide bases either ribonucleotides, or analogs thereof. Target nucleic acids may have three-dimensional structure, may include coding or non-coding regions, may include exons, introns, mRNA, tRNA, rRNA, siRNA, shRNA, miRNA, ribozymes, circular RNA, cDNA, plasmids, vectors, exogenous sequences, endogenous sequences. A target nucleic acid can comprise modified nucleotides, include methylated nucleotides, or nucleotide analogs. In some embodiments, a target nucleic acid may be interspersed with non-nucleic acid components.

A target nucleic acid is recognized by CRISPR-Cas system and binds Cas. In some embodiments, it is modified or cleaved or has altered expression due to the binding of Cas. A target nucleic acid contains a specific recognizable PAM motif.

### Recombinant Gene Technology

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Recombinant expression of a gene, such as a nucleic acid encoding a polypeptide, such as an engineered Cas protein-base editor fusion protein described herein, can include construction of an expression vector containing a nucleic acid that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and the transfected cells can then be cultured by conventional techniques to produce polypeptides.

In some embodiments, a nucleotide sequence encoding a targeting RNA and/or Cas protein is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, the eukaryotic cell is a human cell. In some embodiments, a nucleotide sequence encoding a targeting RNA and/or a novel Cas protein is operably linked to multiple control elements that allow expression of the encoded nucleotide sequence in both prokaryotic and eukaryotic cells.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31 (17)), and/or a human HI promoter (HI).

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), Steroid-regulated promoter, Metal-regulated promoter, estrogen receptor-regulated promoter, etc. Inducible promoters can therefore be regulated by molecules including, but not limited to, doxycycline, RNA polymerase, e.g., T7 RNA polymerase, an estrogen receptor and/or an estrogen receptor fusion.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. Thus, a spatially restricted promoter can be used to regulate the expression of a nucleic acid encoding a subject site-directed polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter, an aromatic amino acid decarboxylase (AADC) promoter, a neurofilament promoter, a synapsin promoter, a thy-1 promoter, a serotonin receptor promoter, a tyrosine hydroxylase promoter (TH), a GnRH promoter, an L7 promoter, a DNMT promoter, an enkephalin promoter, a myelin basic protein (MBP) promoter, a Ca²⁺-calmodulin-dependent protein kinase II-alpha (CamKIIa) promoter and/or a CMV enhancer/platelet-derived growth factor-β promoter.

Adipocyte-specific spatially restricted promoters include, but are not limited to aP2 gene promoter/enhancer, e.g., a region from −5.4 kb to +21 bp of a human aP2 gene, a glucose transporter-4 (GLUT4) promoter, a fatty acid translocase (FAT/CD36) promoter, a stearoyl-CoA desaturase-1 (SCD1) promoter, a leptin promoter, and an adiponectin promoter, an adipsin promoter and/or a resistin promoter.

Cardiomyocyte-specific spatially restricted promoters include, but are not limited to control sequences derived from the following genes: myosin light chain-2, a-myosin heavy chain, AE3, cardiac troponin C, and/or cardiac actin.

Smooth muscle-specific spatially restricted promoters include, but are not limited to an SM22a promoter, a smoothelin promoter, and/or an a-smooth muscle actin promoter.

Photoreceptor-specific spatially restricted promoters include, but are not limited to, a rhodopsin promoter, a rhodopsin kinase promoter, a beta phosphodiesterase gene promoter, a retinitis pigmentosa gene promoter, an interphotoreceptor retinoid-binding protein (IRBP) gene enhancer, and/or an IRBP gene promoter.

In some embodiments, a T7 promoter is used. In some embodiments, a cardiac specific promoter is used. In some embodiments, an inducible promoter is used.

### Therapeutic Applications

The CRISPR-Cas methods or systems described herein can have various therapeutic applications. Accordingly, in some embodiments, a method of treating a disorder or a disease in a subject in need thereof is provided, the method comprising administering to the subject a CRISPR-Cas system comprising a Cas as described herein, wherein the guide RNA is complementary to at least 10 nucleotides of a target nucleic acid associated with the condition or disease; wherein the Cas protein associates with the guide RNA; wherein the guide RNA binds to the target nucleic acid; wherein the Cas protein causes a break in the target nucleic acid, optionally wherein the Cas is an inactive Cas (dCas) fused to a deaminase and results in one or more base edits in the target nucleic acid, thereby treating the disorder or disease.

In some embodiments, the CRISPR-Cas methods or systems can be used to treat various diseases and disorders, e.g., genetic disorders (e.g., monogenetic diseases), diseases that can be treated by nuclease activity, and various cancers, etc.

In some embodiments, the CRISPR methods or systems described herein can be used to edit a target nucleic acid to modify the target nucleic acid (e.g., by inserting, deleting, or mutating one or more nucleic acid residues). For example, in some embodiments the CRISPR systems described herein comprise an exogenous donor template nucleic acid (e.g., a RNA molecule), which comprises a desirable nucleic acid sequence. Upon resolution of a cleavage event induced with the CRISPR system described herein, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the CRISPR systems described herein may be used to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). In some embodiments, the CRISPR methods or systems described herein comprise a nucleobase editor. For example, in some embodiments, the RanCas13 or PspCas13 described herein is fused to a polypeptide having nucleobase editing activity.

CRISPR methods or systems can be used for treating a disease caused by overexpression of RNAs, toxic RNAs, and/or mutated RNAs (e.g., splicing defects or truncations). In some embodiments, the CRISPR methods or systems described herein can also target trans-acting mutations affecting RNA-dependent functions that cause various diseases. In some embodiments, the CRISPR methods or systems described herein can also be used to target mutations disrupting the cis-acting splicing codes that can cause splicing defects and diseases.

The CRISPR methods or systems described herein can further be used for antiviral activity, in particular against RNA viruses. The CRISPR-associated proteins can target the viral RNAs using suitable RNA guides selected to target viral RNA sequences.

The CRISPR methods or systems described herein can also be used to treat a cancer in a subject (e.g., a human subject). For example, the CRISPR-associated proteins described herein can be programmed with crRNA targeting a RNA molecule that is aberrant (e.g., comprises a point mutation or are alternatively-spliced) and found in cancer cells to induce cell death in the cancer cells (e.g., via apoptosis).

Further, the CRISPR methods or systems described herein can also be used to treat an infectious disease in a subject. For example, the CRISPR-associated proteins described herein can be programmed with crRNA targeting a RNA molecule expressed by an infectious agent (e.g., a bacteria, a virus, a parasite or a protozoan) in order to target and induce cell death in the infectious agent cell. The CRISPR systems may also be used to treat diseases where an intracellular infectious agent infects the cells of a host subject. By programming the CRISPR-associated protein to target a RNA molecule encoded by an infectious agent gene, cells infected with the infectious agent can be targeted and cell death induced.

Furthermore, in vitro RNA sensing assays can be used to detect specific RNA substrates. The CRISPR-associated proteins can be used for RNA-based sensing in living cells. Examples of applications are diagnostics by sensing of, for examples, disease-specific RNAs.

In some embodiments, RNA is transiently, stably or inducibly modified, for ex vivo therapy. In some embodiments, as when a selectable marker has been inserted into the nucleic acid region of interest, the population of cells may be enriched for those comprising the RNA modification by separating the cells from the remaining population. Prior to enriching, the cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell compositions that are highly enriched for cells comprising modified RNA are achieved in this manner. By “highly enriched”, it is meant that the modified cells will be 70% or more, 75% or more, 80% or more, 85% or more, 90% or more of the cell composition, for example, about 95% or more, or 98% or more of the cell composition. In other words, the composition may be a substantially pure composition of modified cells.

Modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%),

L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been modified in this way may be transplanted to a subject for, e.g. to treat a disease or as an antiviral, antipathogenic, or anticancer therapeutic or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mouse, rat, guinea pig, hamster, lagomorpha (e.g., rabbit), etc.) may be used for experimental investigations.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Cells may also be introduced into an embryo (e.g., a blastocyst) for the purpose of generating a transgenic animal (e.g., a transgenic mouse).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the invention, site-directed modifying polynucleotide is employed to modify RNA in vivo, e.g. to treat a disease or as an antiviral, antipathogenic, or anticancer therapeutic, for the production of genetically modified organisms in agriculture, or for biological research. In these in vivo embodiments, the RNA modifying polynucleotide is administered directly to the individual. A RNA targeting polynucleotide or polypeptide comprising a fusion of a base editor with Cas is administered by any of a number of well-known methods in the art for the administration of peptides, small molecules and nucleic acids to a subject. A site-directed modifying polypeptide and/or polynucleotide can be incorporated into a variety of formulations. More particularly, a site-directed modifying polypeptide and/or polynucleotide of the present invention can be formulated into pharmaceutical compositions by combination with appropriate pharmaceutically acceptable carriers or diluents.

Pharmaceutical preparations are compositions that include one or more targeting RNA and/or site-directed modifying polypeptide and/or donor polynucleotide present in a pharmaceutically acceptable vehicle. “Pharmaceutically acceptable vehicles” may be vehicles approved by a regulatory agency of the Federal or a state government or listed in the U.S.

Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of a targeting RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel.

Alternatively, drug delivery of therapeutics agents behind the BBB may be by local delivery, for example by intrathecal delivery.

Typically, an effective amount of a RNA and/or site-directed modifying polypeptide and/or donor polynucleotide are provided. As discussed above with regard to ex vivo methods, an effective amount or effective dose of a targeting RNA and/or site-directed modifying polypeptide and/or donor polynucleotide in vivo is the amount to induce a 2 fold increase or more in the amount of editing relative to a negative control, e.g. a cell contacted with an empty vector or irrelevant polypeptide. The amount of recombination may be measured by any convenient method, e.g. as described above and known in the art. The calculation of the effective amount or effective dose of a targeting RNA and/or site-directed modifying polypeptide and/or donor polynucleotide to be administered is within the skill of one of ordinary skill in the art, and will be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a targeting RNA and/or site-directed modifying polypeptide and/or donor polynucleotide may be obtained from a suitable commercial source. As a general proposition, the total pharmaceutically effective amount of the a targeting RNA and/or site-directed modifying polypeptide and/or donor polynucleotide administered parenterally per dose will be in a range that can be measured by a dose response curve.

Therapies based on a targeting RNA and/or site-directed modifying polypeptide and/or donor polynucleotides, i.e. preparations of a targeting RNA and/or site-directed modifying polypeptide and/or donor polynucleotide to be used for therapeutic administration, must be sterile. Sterility is readily accomplished by filtration through sterile filtration membranes (e.g., 0.2 μηt membranes). Therapeutic compositions generally are placed into a container having a sterile access port, for example, an intravenous solution bag or vial having a stopper pierceable by a hypodermic injection needle. The therapies based on a targeting RNA and/or site-directed modifying polypeptide and/or donor polynucleotide may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Delivery Systems

The CRISPR systems described herein, or components thereof, nucleic acid molecules thereof, and/or nucleic acid molecules encoding or providing components thereof, CRISPR-associated proteins, or RNA guides, can be delivered by various delivery systems such as vectors, e.g., plasmids and delivery vectors. Exemplary embodiments are described below. The CRISPR systems (e.g., including the Cas comprising nucleobase editor described herein) can be encoded on a nucleic acid that is contained in a viral vector. Viral vectors can include lentivirus, Adenovirus, Retrovirus, and Adeno-associated viruses (AAVs). Viral vectors can be selected based on the application. For example, AAVs are commonly used for gene delivery in vivo due to their mild immunogenicity. Adenoviruses are commonly used as vaccines because of the strong immunogenic response they induce. Packaging capacity of the viral vectors can limit the size of the base editor that can be packaged into the vector. For example, the packaging capacity of the AAVs is ˜4.5 kb including two 145 base inverted terminal repeats (ITRs).

AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein of the invention and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289 (21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, the CRISPR system of the invention can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In some embodiments, a portion or fragment of a nuclease (e.g., Cas13) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

The disclosed strategies for designing CRISPR systems including the Cas13 described herein can be useful for generating CRISPR systems capable of being packaged into a viral vector. The use of RNA or DNA viral based systems for the delivery of a base editor takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a CRISPR system (e.g., including the Cas13 disclosed herein) of the present disclosure is of sufficient size so as to enable efficient packaging and delivery into a target cell via a retroviral vector. In some cases, a Cas13 is of a size so as to allow efficient packing and delivery even when expressed together with a guide nucleic acid and/or other components of a targetable nuclease system.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

A CRISPR system (e.g., including the Cas13 disclosed herein) described herein can therefore be delivered with viral vectors. One or more components of the base editor system can be encoded on one or more viral vectors. For example, a base editor and guide nucleic acid can be encoded on a single viral vector. In other cases, the base editor and guide nucleic acid are encoded on different viral vectors. In either case, the base editor and guide nucleic acid can each be operably linked to a promoter and terminator.

The combination of components encoded on a viral vector can be determined by the cargo size constraints of the chosen viral vector.

### Non-Viral Delivery of Base Editors

Non-viral delivery approaches for CRISPR are also available. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 10 (below).

TABLE 10
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 11 lists exemplary polymers for use in gene transfer and/or nanoparticle formulations.

TABLE 11
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 12 summarizes delivery methods for a polynucleotide encoding a Cas13 described herein.

TABLE 12

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of genome editing system components or nucleic acids encoding such components, for example, a nucleic acid binding protein such as, for example, Cas13 or variants thereof, optionally fused to a polypeptide having biological activity (e.g., a nucleobase editor), and a gRNA targeting a nucleic acid sequence of interest, may be accomplished by delivering a ribonucleoprotein (RNP) to cells. The RNP comprises the nucleic acid binding protein, e.g., Cas13, in complex with the targeting gRNA. RNPs may be delivered to cells using known methods, such as electroporation, nucleofection, or cationic lipid-mediated methods, for example, as reported by Zuris, J. A. et al., 2015, Nat. Biotechnology, 33 (1): 73-80. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas13 variants).

A promoter used to drive the CRISPR system (e.g., including the Cas13 described herein) can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the Cas and, where appropriate, the guide nucleic acid. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For Osteoblasts suitable promoters can include OG-2.

In some cases, a Cas of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters such as U6 or H1 Use of Pol II promoter and intronic cassettes to express gRNA Adeno Associated Virus (AAV).

A Cas described herein with or without one or more guide nucleic can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome.

AAV has a packaging limit of 4.5 or 4.75 Kb. Constructs larger than 4.5 or 4.75 Kb can lead to significantly reduced virus production.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82:5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 μl Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example a guide RNA or a Cas-encoding mRNA, can be delivered in the form of RNA. Cas encoding mRNA can be generated using in vitro transcription. For example, Cas mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the Cas sequence and/or the guide nucleic acid can be modified to include one or more modified nucleoside e.g. using pseudo-U or 5-Methyl-C.

The disclosure in some embodiments comprehends a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the base editors, compositions and methods of the present disclosure can be such that the cell and progeny of the cell are altered for improved production of biologic products such as an antibody, starch, alcohol or other desired cellular output. The modification introduced to the cell by the methods of the present disclosure can be such that the cell and progeny of the cell include an alteration that changes the biologic product produced.

The system can comprise one or more different vectors. In an aspect, the Cas is codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/(visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Pharmaceutical Compositions

Other aspects of the present disclosure relate to pharmaceutical compositions comprising CRISPR system (e.g., including Cas fused to a base editor as disclosed herein). The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for RNA editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et ah, 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6:1438-47). Positively charged lipids such as N-[1-(2,3-dioleoyloxi) propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the CRISPR system (e.g., including the Cas described herein) are provided as part of a pharmaceutical composition. In some embodiments, the pharmaceutical composition comprises any of the fusion proteins provided herein (e.g., including the nucleobase editor described herein comprising PspCas13 or RanCas13). In some embodiments, the pharmaceutical composition comprises any of the complexes provided herein. In some embodiments, the pharmaceutical composition comprises a ribonucleoprotein complex comprising an RNA-guided nuclease (e.g., Cas13) fused to a RESCUE or REPAIR base editor, that forms a complex with a gRNA and a cationic lipid. In some embodiments pharmaceutical composition comprises a gRNA, a nucleic acid programmable RNA binding protein, a cationic lipid, and a pharmaceutically acceptable excipient. Pharmaceutical compositions can optionally comprise one or more additional therapeutically active substances.

### Kits

In one aspect, the invention provides kits containing any one or more of the elements disclosed in the above methods and compositions. In some embodiments, the kit comprises a vector system and instructions for using the kit. In some embodiments, the vector system comprises one or more insertion sites for inserting a guide sequence, wherein when expressed, the guide sequence directs sequence-specific binding of a CRISPR complex to a target mRNA sequence in a eukaryotic cell, wherein the CRISPR complex comprises a CRISPR enzyme complexed with (1) the guide sequence that is hybridized to the target sequence, and (2) a Cas protein; and/or (b) a base editor operably linked to an enzyme-coding sequence encoding said Cas protein comprising a nuclear localization sequence. Elements may be provide individually or in combinations, and may be provided in any suitable container, such as a vial, a bottle, or a tube. In some embodiments, the kit includes instructions in one or more languages, for example in more than one language.

In some embodiments, the kit comprises a nucleobase editor. For example, in some embodiments, the kit includes a nucleobase editor comprising the Prevotella sp. or Riemerella anatipestifer Cas13 described herein.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1. RNA Editing to Target Phosphorylation of Yes-Associated Protein 1 (YAP1) or TAZ in the Hippo Pathway

This example describes RNA editing of exemplary YAP1 mRNA to manipulate the Hippo pathway through targeting exemplary YAP1 phosphorylation sites, S127 (mouse S112) and S109 (mouse S94). A similar approach is used for site-specific TAZ transcriptional coactivator. Exemplary phosphorylation sites targeted include one or more YAP1 phosphorylation sites selected from S127, S109, S164, S381, S383 and S384 and one or more TAZ phosphorylation sites, selected from S89, S314, S311, S117 and S66.

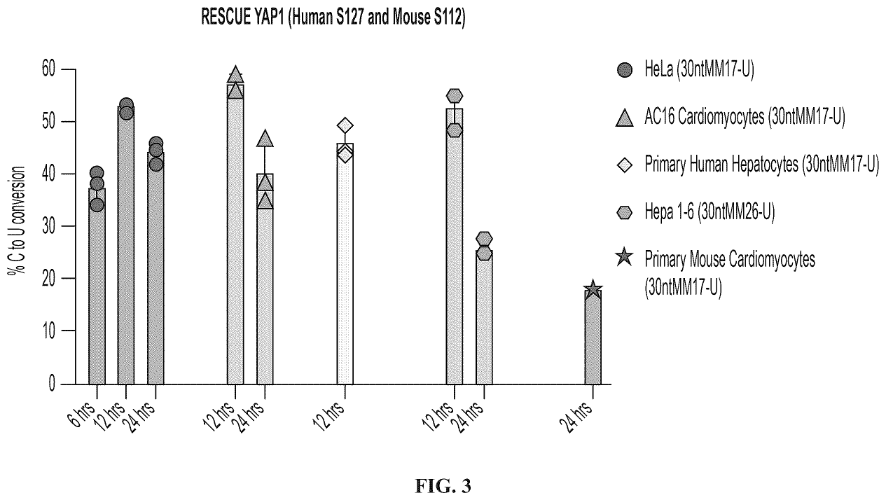

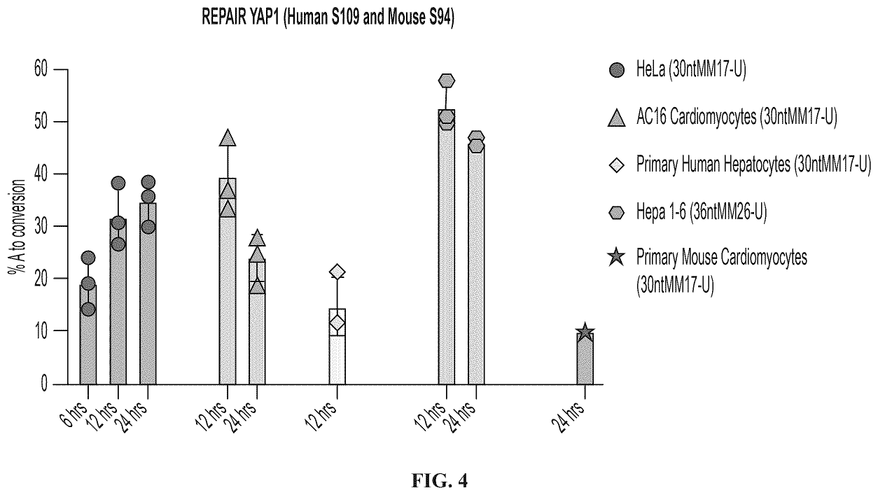

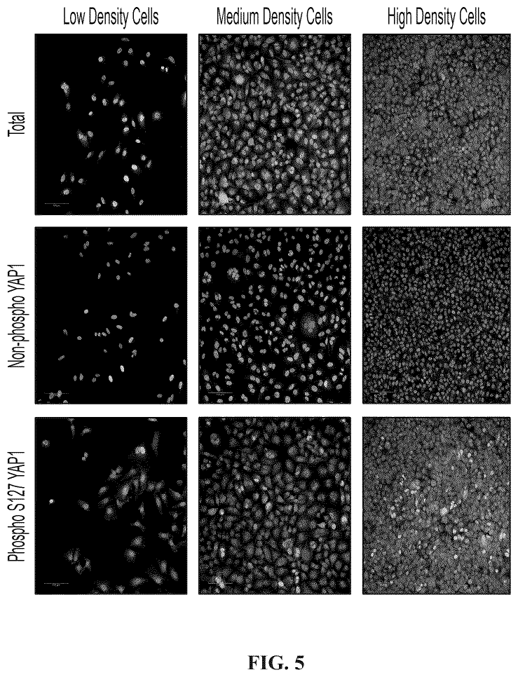

Briefly, guide RNAs were designed containing a spacer region of 30 nucleotides in length with a mismatch, either 17 nucleotides in length (30ntMM17-U, FIGS. 3 and 4), 24 nucleotides in length (e.g. TAZg24_S314MM17-C, Table 2 and 3), 25 nucleotides in length (e.g. S164 30ntMM25-C, Table 2 and 3) or 26 nucleotides in length (30ntMM26-U, FIGS. 3 and 4) from the scaffold targeting YAP1 or TAZ. Table 2 shows guide RNA sequences targeting YAP1 and TAZ RNA. Table 3 shows guide RNA sequences targeting YAP1 and TAZ RNA including scaffold and extension sequences. Sequences are shown from 5′ to 3′.

RESCUE and REPAIR base editing systems were used to target exemplary YAP1 or TAZ phosphorylation sites. e.g. REPAIR base editor Psp dCas13b-hADAR2 deaminase domain (SEQ ID NO: 1) that carries out A to I editing; and RESCUE base editors Ran dCas13b-hADAR2 (SEQ ID NO: 2) and Psp dCas13b-hADAR2 (SEQ ID NO: 3), which are evolved base editors that carry out C-to-U editing, in addition to A-to-I editing, were used for YAP1 RNA targeting. The same approach is also used for TAZ RNA targeting.

A variety of cell lines were tested, for example, HeLa, AC16 cardiomyocytes, primary human hepatocytes, Hepa 1-6 and primary mouse cardiomyocytes.

Briefly, for example, about 25,000 cells were plated per 96-well. Cells were transfected with 100 ng of Cas-fused to base editor, and 100 ng of guide RNA, 24 h after plating. Cells were harvested at various timepoints after transfection and RNA was analyzed. Sequencing was carried out to characterize C-to-U and A-to-I editing in cells.

The results showed that percentage C to U editing by targeting serine 127 using a RESCUE editor for RNA editing was about 50-60% editing in non-primary cell lines, and about 45% editing in primary human hepatocytes at 12h post-transfection (FIG. 3). Editing in primary mouse cardiomyocytes was about 18% at 24h.

Evaluation of percentage A to I editing by targeting serine 109 using a REPAIR editor for RNA editing resulted in about 30-50% editing in non-primary cell lines, and about 15% editing in primary human hepatocytes at 12h post-transfection (FIG. 4). Editing in primary mouse cardiomyocytes was about 10% at 24h.

Overall, the results from this study showed efficient RNA editing of YAP1 mRNA at exemplary YAP1 phosphorylation sites, thus indicating the feasibility of manipulating signaling via the Hippo pathway.

### Example 2. Localization of Endogenous YAP1 or TAZ by Immunofluorescence

This example illustrates exemplary localization of endogenous YAP1 transcriptional coactivator by immunofluorescence.

Endogenous YAP1 localization was monitored in HeLa cells using immunofluorescence. Antibodies specific for the various phosphorylation states of YAP1 were used, including total YAP, non-phospho (active YAP) and phospho-S127 (inactive YAP) antibodies. Cells were plated at low, medium and high confluencies to simulate the Hippo pathway in its ‘on’ and ‘off’ forms. Without wishing to be bound to any particular theory, in its ‘on’ form, YAP1 protein is phosphorylated at for example, serine 127, and subsequently S109, S164, S381, 383, 384 in the cytosol and YAP1 protein is targeted for degradation. In the ‘off’ form, YAP1 protein is non-phosphorylated, enters the nucleus and interacts with the TEAD family of transcription factors to activate transcription of pathways involved in replication, organ size, stem cell renewal and cell survival such as, but not limited to Wnt, c-Myc, cyclin D, FGF1, CTGF, TGFβ/SMAD (FIG. 1). The TAZ protein functions similarly in the Hippo pathway and its localization is assessed by the same approach.

The results showed nuclear and cytosolic localization in the total YAP staining. Non-phosphorylated (active YAP) localized to the nucleus, while phosphorylated S127 (inactive YAP) localized to the cytosol.

Overall, the results from this experiment showed RNA editing of YAP1 mRNA at exemplary YAP1 phosphorylation sites, thus manipulating signaling via the kinase cascade of Hippo pathway.

### Example 3. In Vivo Targeting of YAP1 or TAZ in Mouse Hearts

This example demonstrates in vivo targeting of YAP1 or TAZ in cardiac tissue.

Briefly, an experimental design for the in vivo tolerability and editing study using different molar ratios of our editor and guide RNA is shown in Table 13.

TABLE 13
Experimental design for in vivo tolerability and editing

| Molar Ratio | No. of mice | No. of mice | Editor | Guide RNA |
|---|---|---|---|---|
| (Editor:Guide) | (12 hours) | (24 hours) | mRNA (μg) | (μg) |
| 1:1 | 6 | 6 | 246 | 4 |
| 1:10 | 6 | 6 | 215 | 35 |
| 1:35 | 6 | 6 | 160 | 90 |
| Mock | 6 | 6 | Saline | Saline |

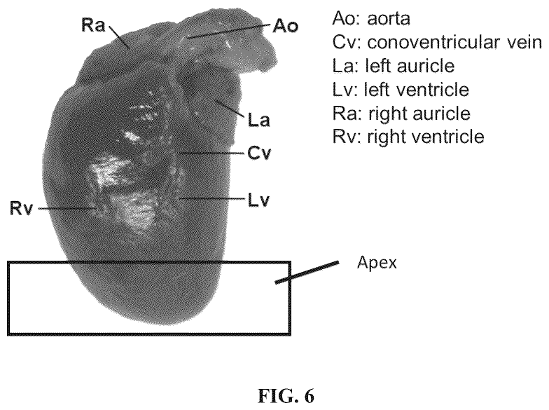

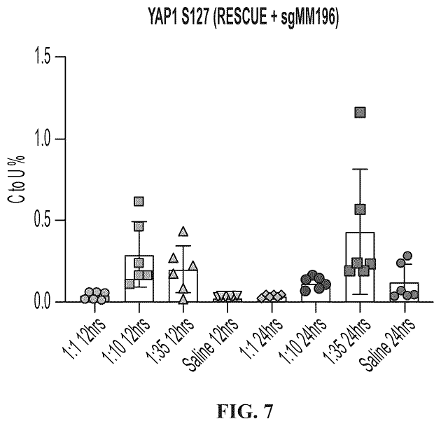

Briefly, six mice in each group were injected with 1:1, 1:10 or 1:35 base editor mRNA: sgRNA (modified) in a total of 250 μg. In the control group, animals were injected with saline. Illustration of a mouse heart is depicted in FIG. 6, and the designated injection site is as shown. Tissue in the apex was injected at two different regions close to each other is injected with 50 μL of editor and guide each formulated in 1×PBS, pH 7.4, then dissected and collected into RNALater at 12 and 24h timepoints. RNA was isolated from the samples to detect RNA editing activity in YAP1 mRNA (Table 13 and 14, FIG. 7). Similarly, samples are processed to detect RNA editing activity in TAZ mRNA.

TABLE 14
C-to-U Editing Efficiency in cardiac tissue, 24 hours after injection

| Sample (Editor:Guide) | C-to-U % | Average |
|---|---|---|
| Sample 1 (1:35) 24 h | 0.57 | 0.434 |
| Sample 2 (1:35) 24 h | 1.162 | |
| Sample 3 (1:35) 24 h | 0.238 | |
| Sample 4 (1:35) 24 h | 0.19 | |
| Sample 5 (1:35) 24 h | 0.199 | |
| Sample 6 (1:35) 24 h | 0.245 | |
| Sample 1 (1:10) 24 h | 0.165 | 0.120333 |
| Sample 2 (1:10) 24 h | 0.111 | |
| Sample 3 (1:10) 24 h | 0.14 | |
| Sample 4 (1:10) 24 h | 0.087 | |
| Sample 5 (1:10) 24 h | 0.071 | |
| Sample 6 (1:10) 24 h | 0.148 | |
| Sample 1 (1:1) 24 h | 0.049 | 0.039 |
| Sample 2 (1:1) 24 h | 0.036 | |
| Sample 3 (1:1) 24 h | 0.048 | |
| Sample 4 (1:1) 24 h | 0.025 | |
| Sample 5 (1:1) 24 h | 0.028 | |
| Sample 6 (1:1) 24 h | 0.048 | |
| Sample 1 (Saline) 24 h | 0.285 | 0.121333 |
| Sample 2 (Saline) 24 h | 0.042 | |
| Sample 3 (Saline) 24 h | 0.241 | |
| Sample 4 (Saline) 24 h | 0.072 | |
| Sample 5 (Saline) 24 h | 0.048 | |
| Sample 6 (Saline) 24 h | 0.04 | |

TABLE 15
C-to-U Editing Efficiency in cardiac tissue, 12 hours after injection

| Sample (Editor:Guide) | C-to-U % | Average |
|---|---|---|
| Sample 1 (1:35) 12 h | 0.224 | 0.203333 |
| Sample 2 (1:35) 12 h | 0.435 | |
| Sample 3 (1:35) 12 h | 0.271 | |
| Sample 4 (1:35) 12 h | 0.026 | |
| Sample 5 (1:35) 12 h | 0.176 | |
| Sample 6 (1:35) 12 h | 0.088 | |
| Sample 1 (1:10) 12 h | 0.615 | 0.293833 |
| Sample 2 (1:10) 12 h | 0.242 | |
| Sample 3 (1:10) 12 h | 0.464 | |
| Sample 4 (1:10) 12 h | 0.165 | |
| Sample 5 (1:10) 12 h | 0.113 | |
| Sample 6 (1:10) 12 h | 0.164 | |
| Sample 1 (1:1) 12 h | 0.064 | 0.043167 |
| Sample 2 (1:1) 12 h | 0.058 | |
| Sample 3 (1:1) 12 h | 0.062 | |
| Sample 4 (1:1) 12 h | 0.023 | |
| Sample 5 (1:1) 12 h | 0.022 | |
| Sample 6 (1:1) 12 h | 0.03 | |
| Sample 1 (Saline) 12 h | 0.023 | 0.0275 |
| Sample 1 (Saline) 12 h | 0.022 | |
| Sample 2 (Saline) 12 h | 0.036 | |
| Sample 3 (Saline) 12 h | 0.03 | |
| Sample 4 (Saline) 12 h | 0.033 | |
| Sample 5 (Saline) 12 h | 0.021 | |

The results showed that in mice injected with 1:35 editor:guide, an editing efficiency of about 0.5% was observed at S127 of YAP1, 24 hours after injection (FIG. 7).

Overall, the results showed successful in vivo delivery and in vivo RNA editing of YAP1 mRNA. Resultant effects on protein localization and associated cardiac phenotypes in a mouse model are subsequently assessed.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A guide RNA, comprising:
(a) a scaffold for binding a nucleic acid programmable RNA binding protein; and
(b) a spacer sequence having one or more regions complementary to a target mRNA encoding a Yes-associated protein 1 (YAP1) or a Transcriptional co-activator with PDZ-binding motif (TAZ or WWTR1);
wherein the spacer sequence comprises a single nucleotide mismatch to an adenosine or cytosine in the target mRNA.

2. The guide of claim 1, wherein the nucleic acid programmable RNA binding protein is a Cas protein, Type VI Cas protein, Cas13 protein, or Cas13b protein.

3. The guide RNA of claim 1, wherein the spacer comprises (a) between 4-15 consecutive nucleotides that are perfectly complementary to a target mRNA prior to the mismatch within the spacer; (b) between 16-25 consecutive nucleotides that are perfectly complementary to a target mRNA after the mismatch within the spacer; and/or (c) between 28-35 non-consecutive nucleotides that are perfectly complementary to a target mRNA.

4. (canceled)

5. (canceled)

6. The guide RNA of claim 1, wherein the spacer sequence comprises a sequence having at least 80% or greater identity to a sequence selected from the group consisting of SEQ ID NOs: 7-33 and the guide RNA comprises a sequence having at least 80% or greater identity to a sequence selected from the group consisting of SEQ ID NOs: 34-60.

7-9. (canceled)

10. The guide RNA of claim 1, wherein the guide RNA is (a) bound to a base editor, (b) bound to an mRNA encoding YAP1 or TAZ, and/or (c) comprises a region that binds to an ADAR protein.

11-13. (canceled)

14. The guide RNA of claim 1, wherein the guide RNA comprises:
(a) a spacer sequence from about 30-36 nucleotides in length;
(b) a mismatch about 17, 24, 25 or 26 nucleotides from the 5′ or 3′ end of the spacer sequence;
(c) a chemical modification at a 5′ terminal nucleotide and/or a 3′ terminal nucleotide of the guide RNA;
(d) 3X 2′O-methyl and/or phosphorothioate at 5′ and/or 3′ end; and/or
(e) a 6 nucleotide extension sequence.

15-19. (canceled)

20. The guide RNA of claim 14, wherein the extension sequence comprises UUmC*mG*mA*U (SEQ ID NO: 4), wherein mC* refers to 2′O-methyl cytosine, mG* refers to 2′O-methyl guanine and mA* refers to 2′O-methyl adenine.

21. The guide RNA of claim 1, wherein the scaffold sequence is derived from Prevotella sp. or Riemerella antaipestifer and comprises a sequence having at least 80% identity to SEQ ID NO.: 5 or 6.

22-25. (canceled)

26. An engineered, non-naturally occurring composition for modifying a target RNA base, comprising:
(a) a scaffold for binding a nucleic acid programmable RNA binding protein,
(b) a base editor, and/or
(c) a guide RNA molecule with a spacer sequence having one or more regions complementary to a mRNA encoding Yes-associated protein 1 (YAP1) or Transcriptional co-activator with PDZ-binding motif (TAZ or WWTR1).

27. The composition of claim 26, wherein the nucleic acid programmable RNA binding protein comprises a Cas protein, a Type VI Cas protein, Cas13 protein, Cas13b protein, a catalytically inactive Cas13 protein, or a dead Cas13 protein.

28-40. (canceled)

41. The composition of claim 26, wherein the base editor comprises an adenine or cytosine deaminase with at least 80% identity to a nucleic acid sequence of SEQ ID NO: 1-3.

42-45. (canceled)

46. The composition of claim 26, wherein the guide RNA comprises a sequence having complementarity to a target mRNA sequence that comprises an adenine or cytidine, and wherein the spacer sequence comprises a single nucleotide mismatch corresponding to adenosine or cytidine in the target mRNA.

47-50. (canceled)

51. The composition of claim 26, wherein the guide RNA comprises
(a) a spacer sequence from about 30-36 nucleotides in length;
(b) a mismatch about 17, 24, 25 or 26 nucleotides from the 5′ or 3′ end of the spacer sequence;
(c) 3X 2′O-methyl and/or phosphorothioate at 5′ and/or 3′ end; and/or
(d) a 6 nucleotide extension sequence comprising SEQ ID NO: 4.

52-55. (canceled)

56. The composition of claim 26, wherein the scaffold sequence comprises a Prevotella sp. scaffold of SEQ ID NO: 5 or a Riemerella anatipestifer scaffold of SEQ ID NO: 6.

57. (canceled)

58. The composition of claim 26, wherein the modification of targeted mRNA base changes:
(a) one or more post-translational modification sites of the protein encoded by the target RNA;
(b) a single phosphorylation site in the protein encoded by the target mRNA;
(c) multiple phosphorylation sites of the protein encoded by the target RNA; and/or
(d) the encoded amino acid from a serine, threonine, or tyrosine to an amino acid that cannot be phosphorylated.

59-73. (canceled)

74. The composition of claim 26, wherein the target RNA base modification modifies one or more YAP1 phosphorylation sites selected from S127, S109, S164, S381, S383 and S384 and/or one or more TAZ phosphorylation sites, selected from S89, S314, S311, S117 and S66.

75-83. (canceled)

84. An engineered, non-naturally occurring system for RNA editing, comprising the composition of claim 26.

85. A method of modifying a target RNA base, the method comprising administering a composition comprising:
(a) a nucleic acid programmable RNA binding protein,
(b) a base editor, and
(c) a guide RNA molecule directed to an mRNA encoding YAP1 or TAZ,
wherein the administering modifies the target mRNA base.

86. The method of claim 85, wherein the method comprises administering a composition comprising: the nucleic acid programmable RNA binding protein, a RESCUE or REPAIR base editor, and a guide RNA molecule;
wherein the modifying of target mRNA base is from adenosine to inosine or cytidine to uracil; and
wherein the RNA base modification results in a modified phosphorylation site of YAP1 or TAZ protein.

87-104. (canceled)

105. A method of treating disease by administering to a subject in need thereof, an effective amount of the composition of claim 26, wherein the composition activates or inactivates a signaling pathway by a post-translational modification.

106-120. (canceled)


SEQUENCE LISTING (142 sequences)

SEQ ID NO: 1 - protein, 4495 aa, Prevotella sp.
ATGAATATCCCCGCCCTGGTGGAAAACCAGAAGAAATACTTTGGAACTTACTCCGTGATGGCTATGCTGAATGCTCAGACCGTGCTGGATCACATCCAGAAGGTGGCCGACATCGAGGGCGAGCAGAACGAGAACAATGAGAATCTGTGGTTCCACCCCGTGATGAGCCACCTGTACAACGCCAAGAATGGCTATGATAAGCAGCCTGAAAAGACCATGTTTATCATCGAGCGCCTGCAGTCCTATTTCCCATTTCTGAAGATCATGGCCGAGAATCAGAGAGAGTACTCTAACGGCAAGTATAAGCAGAATAGGGTGGAGGTGAACAGCAATGACATCTTCGAGGTGCTGAAGCGCGCCTTTGGCGTGCTGAAGATGTACCGGGATCTGACCAACGCCTACAAGACCTACGAGGAGAAGCTGAATGACGGCTGCGAGTTCCTGACCTCCACAGAGCAGCCACTGTCTGGCATGATCAACAATTACTATACCGTGGCCCTGAGAAACATGAATGAGAGGTACGGCTACAAGACAGAGGATCTGGCCTTTATCCAGGACAAGCGGTTCAAGTTTGTGAAGGACGCCTACGGCAAGAAGAAGAGCCAGGTGAACACCGGCTTCTTTCTGTCTCTGCAGGATTACAATGGCGACACACAGAAGAAGCTGCACCTGAGCGGAGTGGGAATCGCCCTGCTGATCTGCCTGTTCCTGGATAAGCAGTATATCAACATCTTCCTGAGCCGCCTGCCCATCTTTAGCTCCTACAATGCCCAGTCCGAGGAGCGGAGAATCATCATCCGGAGCTTCGGCATCAACTCCATCAAGCTGCCAAAGGACAGAATCCACTCTGAGAAGAGCAACAAGTCCGTGGCTATGGACATGCTGAATGAGGTGAAGCGGTGTCCCGACGAGCTGTTTACCACACTGTCTGCCGAGAAGCAGAGCCGGTTCAGAATCATCTCCGACGATCACAACGAGGTGCTGATGAAGCGGAGCAGCGACCGGTTCGTGCCTCTGCTGCTGCAGTACATCGATTATGGCAAGCTGTTCGACCACATCAGATTTCACGTGAACATGGGCAAGCTGAGATACCTGCTGAAGGCCGATAAGACCTGCATCGACGGCCAGACAAGGGTGCGCGTGATCGAGCAGCCACTGAATGGCTTCGGCAGACTGGAGGAGGCCGAGACAATGAGGAAGCAGGAGAACGGCACATTTGGCAATAGCGGCATCCGGATCAGAGATTTCGAGAACATGAAGAGAGACGATGCCAACCCTGCCAATTACCCATATATCGTGGACACCTACACACACTATATCCTGGAGAACAATAAGGTGGAGATGTTCATCAATGATAAGGAGGACTCCGCCCCACTGCTGCCTGTGATCGAGGACGATCGCTATGTGGTGAAGACCATCCCCAGCTGTCGGATGTCCACACTGGAGATCCCTGCTATGGCCTTTCACATGTTCCTGTTTGGCAGCAAGAAGACCGAGAAGCTGATCGTGGACGTGCACAACCGGTACAAGAGACTGTTTCAGGCCATGCAGAAGGAGGAGGTGACAGCCGAGAATATCGCCAGCTTCGGCATCGCCGAGTCCGATCTGCCTCAGAAGATCCTGGACCTGATCTCCGGCAACGCCCACGGCAAGGATGTGGACGCCTTTATCAGACTGACCGTGGACGATATGCTGACCGATACAGAGAGGCGCATCAAGAGGTTCAAGGACGATAGAAAGTCTATCAGGAGCGCCGACAATAAGATGGGCAAGCGCGGCTTTAAGCAGATCAGCACCGGCAAGCTGGCCGATTTCCTGGCCAAGGACATCGTGCTGTTTCAGCCCAGCGTGAACGATGGCGAGAATAAGATCACAGGCCTGAACTACCGGATCATGCAGTCCGCCATCGCCGTGTACGACTCTGGCGACGATTATGAGGCCAAGCAGCAGTTCAAGCTGATGTTTGAGAAGGCCAGACTGATCGGCAAGGGCACCACAGAGCCTCACCCATTCCTGTATAAGGTGTTTGCCAGGTCCATCCCTGCCAACGCCGTGGAGTTTTACGAGAGATACCTGATCGAGCGGAAGTTCTACCTGACCGGCCTGTCTAACGAGATCAAGAAGGGCAATAGAGTGGATGTGCCCTTCATCCGGCGGGACCAGAATAAGTGGAAGACCCCTGCCATGAAGACACTGGGCAGAATCTACAGCGAGGATCTGCCCGTGGAGCTGCCTAGGCAGATGTTCGACAACGAGATCAAGTCTCACCTGAAGAGCCTGCCCCAGATGGAGGGCATCGACTTTAACAATGCCAATGTGACCTACCTGATCGCCGAGTATATGAAGAGGGTGCTGGACGATGACTTCCAGACCTTCTACCAGTGGAACAGGAATTACCGCTATATGGATATGCTGAAGGGCGAGTACGACCGCAAGGGCAGCCTGCAGCACTGCTTCACCTCCGTGGAGGAGAGGGAGGGCCTGTGGAAGGAGAGGGCCTCCAGAACAGAGCGGTACAGAAAGCAGGCCTCCAACAAGATCAGGTCTAATCGCCAGATGCGGAACGCCTCCTCTGAGGAGATCGAGACAATCCTGGATAAGAGACTGAGCAACTCCAGGAATGAGTACCAGAAGAGCGAGAAAGTGATCAGGCGCTATAGAGTGCAGGACGCCCTGCTGTTCCTGCTGGCCAAGAAGACCCTGACAGAGCTGGCCGATTTCGACGGCGAGAGGTTTAAGCTGAAGGAGATCATGCCAGACGCCGAGAAGGGCATCCTGTCTGAGATCATGCCCATGAGCTTCACCTTTGAGAAGGGCGGCAAGAAGTATACCATCACAAGCGAGGGCATGAAGCTGAAGAACTACGGCGATTTCTTTGTGCTGGCCTCCGACAAGAGAATCGGCAATCTGCTGGAGCTGGTGGGCTCCGACATCGTGAGCAAGGAGGACATCATGGAGGAGTTCAACAAGTATGATCAGTGTAGGCCCGAGATCAGCTCCATCGTGTTCAATCTGGAGAAGTGGGCCTTTGACACCTACCCTGAGCTGTCCGCCAGAGTGGATAGGGAGGAGAAGGTGGACTTTAAGTCTATCCTGAAGATCCTGCTGAACAATAAGAACATCAATAAGGAGCAGTCCGACATCCTGCGCAAGATCCGGAACGCCTTCGATGCCAACAATTATCCAGACAAGGGCGTGGTGGAGATCAAGGCCCTGCCCGAGATCGCCATGTCTATCAAGAAGGCCTTTGGCGAGTACGCCATCATGAAGGGCAGCCTGCAGCTGCCACCTCTGGAGCGCCTGACCCTGGGAAGCGGAGGAGGAGGCTCCCAGCTGCACCTGCCTCAGGTGCTGGCCGACGCCGTGTCTCGGCTGGTGCTGGGCAAGTTCGGCGATCTGACCGACAACTTTTCTAGCCCACACGCAAGGAGAAAGGTGCTGGCCGGAGTGGTCATGACCACAGGCACAGATGTGAAGGACGCCAAAGTGATCAGCGTGAGCACCGGCACCAAGTGCATCAACGGCGAGTACATGAGCGATAGAGGCCTGGCCCTGAATGACTGTCACGCCGAGATCATCTCCAGGCGCTCTCTGCTGCGCTTCCTGTACACCCAGCTGGAGCTGTATCTGAACAATAAGGATGACCAGAAGCGGAGCATCTTCCAGAAGTCCGAGCGCGGCGGCTTTCGGCTGAAGGAGAACGTGCAGTTCCACCTGTACATCTCCACATCTCCATGCGGCGATGCCAGAATCTTTTCTCCACACGAGCCAATCCTGGAGGAGCCTGCAGACCGCCACCCAAACAGAAAGGCCAGGGGCCAGCTGCGGACCAAGATCGAGAGCGGCCAAGGCACAATCCCCGTGAGGAGCAATGCCTCCATCCAGACCTGGGATGGCGTGCTGCAGGGAGAGCGCCTGCTGACAATGAGCTGTTCCGACAAGATCGCAAGGTGGAACGTGGTGGGCATCCAGGGCTCTCTGCTGAGCATCTTCGTGGAGCCCATCTACTTTTCCTCTATCATCCTGGGCTCTCTGTATCACGGCGATCACCTGAGCCGCGCCATGTACCAGCGGATCTCCAATATCGAGGACCTGCCACCCCTGTATACCCTGAACAAGCCTCTGCTGTCCGGCATCTCTAATGCAGAGGCAAGGCAGCCAGGCAAGGCACCAAACTTCTCTGTGAATTGGACAGTGGGCGATAGCGCCATCGAAGTGATCAACGCCACCACAGGCAAGGACGAGCTGGGAAGGGCCTCCCGGCTGTGCAAGCACGCCCTGTACTGTAGATGGATGAGGGTGCACGGCAAGGTGCCTTCTCACCTGCTGCGCAGCAAGATCACCAAGCCAAACGTGTACCACGAGAGCAAGCTGGCCGCAAAGGAGTACCAGGCAGCAAAGGCAAGGCTGTTCACAGCCTTTATCAAGGCAGGGCTGGGGGCTTGGGTGGAAAAACCTACCGAGCAGGACCAGTTCTCACTGACATAASEQIDNO

SEQ ID NO: 2 - protein, 4491 aa, Riemerella anatipestifer
ATGGAGAAGCCCCTGCTGCCTAACGTGTATACCCTGAAGCACAAGTTCTTTTGGGGCGCCTTCCTGAATATCGCCCGGCACAACGCCTTTATCACCATCTGCCACATCAATGAGCAGCTGGGCCTGAAAACCCCCAGCAACGACGATAAGATCGTGGACGTGGTGTGCGAGACATGGAACAATATCCTGAACAATGACCACGATCTGCTGAAGAAGTCCCAGCTGACCGAGCTGATCCTGAAGCACTTCCCTTTTCTGACAGCCATGTGCTACCACCCCCCTAAGAAGGAGGGCAAGAAGAAGGGCCACCAGAAGGAGCAGCAGAAGGAGAAGGAGAGCGAGGCACAGTCCCAGGCAGAGGCCCTGAACCCATCCAAGCTGATCGAGGCCCTGGAGATCCTGGTGAATCAGCTGCACTCTCTGGCAAACTACTATAGCGCCTATAAGCACAAGAAGCCCGACGCCGAGAAGGATATCTTCAAGCACCTGTATAAGGCCTTCGACGCCTCTCTGAGAATGGTGAAGGAGGATTATAAGGCCCACTTCACCGTGAATCTGACAAGGGACTTTGCCCACCTGAACCGCAAGGGCAAGAACAAGCAGGACAATCCCGACTTCAACAGATACAGATTCGAGAAGGATGGCTTCTTTACCGAGTCTGGCCTGCTGTTCTTTACAAATCTGTTTCTGGACAAGAGGGATGCCTATTGGATGCTGAAGAAGGTGTCCGGCTTCAAGGCCTCTCACAAGCAGCGCGAGAAGATGACCACAGAGGTGTTTTGCAGGAGCCGCATCCTGCTGCCCAAGCTGAGGCTGGAGTCCCGCTACGACCACAACCAGATGCTGCTGGATATGCTGTCTGAGCTGAGCAGATGTCCTAAGCTGCTGTATGAGAAGCTGAGCGAGGAGAATAAGAAGCACTTCCAGGTGGAGGCCGACGGCTTTCTGGATGAGATCGAGGAGGAGCAGAACCCATTCAAGGACACCCTGATCCGGCACCAGGATAGATTCCCCTACTTTGCCCTGAGGTATCTGGACCTGAATGAGTCCTTCAAGTCTATCCGCTTTCAGGTGGATCTGGGCACATACCACTATTGTATCTACGACAAGAAGATCGGCGATGAGCAGGAGAAGAGGCACCTGACCCGCACACTGCTGTCCTTCGGCCGGCTGCAGGACTTTACCGAGATCAACAGACCCCAGGAGTGGAAGGCCCTGACCAAGGACCTGGATTACAAGGAGACATCCAATCAGCCTTTCATCTCTAAGACCACACCACACTATCACATCACCGACAACAAGATCGGCTTTCGGCTGGGCACAAGCAAGGAGCTGTACCCCTCCCTGGAGATCAAGGATGGCGCCAATAGAATCGCCAAGTACCCTTATAACTCCGGCTTCGTGGCCCACGCCTTTATCTCTGTGCACGAGCTGCTGCCTCTGATGTTCTACCAGCACCTGACCGGCAAGAGCGAGGACCTGCTGAAGGAGACAGTGCGGCACATCCAGAGAATCTATAAGGACTTCGAGGAGGAGCGGATCAATACCATCGAGGATCTGGAGAAGGCAAACCAGGGCAGACTGCCACTGGGAGCCTTTCCCAAGCAGATGCTGGGCCTGCTGCAGAATAAGCAGCCTGATCTGTCCGAGAAGGCCAAGATCAAGATCGAGAAGCTGATCGCCGAGACAAAGCTGCTGTCTCACAGGCTGAACACAAAGCTGAAGAGCTCCCCAAAGCTGGGCAAGCGGAGAGAGAAGCTGATCAAGACAGGCGTGCTGGCCGACTGGCTGGTGAAGGATTTCATGCGCTTTCAGCCCGTGGCCTACGACGCCCAGAATCAGCCTATCAAGTCTAGCAAGGCCAACTCCACCGAGTTCTGGTTTATCAGGCGCGCCCTGGCCCTGTACGGAGGAGAGAAGAATAGGCTGGAGGGCTATTTCAAGCAGACCAACCTGATCGGCAACACAAATCCACACCCCTTCCTGAACAAGTTTAATTGGAAGGCCTGCAGGAATCTGGTGGATTTCTACCAGCAGTATCTGGAGCAGCGCGAGAAGTTTCTGGAGGCCATCAAGAACCAGCCATGGGAGCCCTACCAGTATTGCCTGCTGCTGAAGATCCCTAAGGAGAACAGGAAGAATCTGGTGAAGGGATGGGAGCAGGGAGGAATCTCTCTGCCACGGGGCCTGTTTACCGAGGCCATCAGAGAGACACTGTCTGAGGACCTGATGCTGAGCAAGCCAATCCGCAAGGAGATCAAGAAGCACGGCCGGGTGGGCTTCATCAGCAGAGCCATCACCCTGTACTTTAAGGAGAAGTATCAGGATAAGCACCAGAGCTTCTACAATCTGTCCTATAAGCTGGAGGCAAAGGCACCACTGCTGAAGCGGGAGGAGCACTACGAGTATTGGCAGCAGAACAAGCCTCAGTCTCCAACCGAGAGCCAGAGGCTGGAGCTGCACACAAGCGACCGCTGGAAGGATTACCTGCTGTATAAGAGATGGCAGCACCTGGAGAAGAAGCTGCGGCTGTACCGGAATCAGGACGTGATGCTGTGGCTGATGACCCTGGAGCTGACAAAGAACCACTTCAAGGAGCTGAACCTGAATTATCACCAGCTGAAGCTGGAGAATCTGGCCGTGAACGTGCAGGAGGCCGATGCCAAGCTGAACCCTCTGAATCAGACCCTGCCCATGGTGCTGCCTGTGAAGGTGTACCCTGCCACAGCCTTTGGCGAGGTGCAGTACCACAAGACCCCAATCAGGACAGTGTATATCCGCGAGGAGCACACCAAGGCCCTGAAGATGGGCAACTTCAAGGCCCTGGTGAAGGACCGGAGACTGAATGGCCTGTTCTCTTTTATCAAGGAGGAGAACGATACACAGAAGCACCCAATCAGCCAGCTGCGGCTGAGGCGCGAGCTGGAGATCTACCAGTCTCTGAGAGTGGACGCCTTCAAGGAGACACTGAGCCTGGAGGAGAAGCTGCTGAATAAGCACACATCCCTGTCCTCTCTGGAGAACGAGTTTAGGGCCCTGCTGGAGGAGTGGAAGAAGGAGTATGCCGCCAGCTCCATGGTGACAGACGAGCACATCGCCTTCATCGCCAGCGTGGCCAATGCCTTCTGCGCCAACCAGTACCCCTTCTACAAGGAGGCCCTGCACGCCCCTATCCCACTGTTCACCGTGGCCCAGCCCACCACAGAGGAGAAGGATGGCCTGGGAATCGCAGAGGCCCTGCTGAAGGTGCTGAGGGAGTACTGTGAGATCGTGAAGTCCCAGATCGGATCCAGCCTCCAAAAAAAGTTGGAGGAATTAGAACTAGGATCCCAGCTGCACCTGCCTCAGGTGCTGGCCGACGCCGTGTCTCGGCTGGTGATCGGCAAGTTCGGCGATCTGACCGACAACTTTTCTAGCCCACACGCAAGGAGAATCGGCCTGGCCGGAGTGGTCATGACCACAGGCACAGATGTGAAGGACGCCAAAGTGATCTGCGTGAGCACCGGCAGCAAGTGCATCAACGGCGAGTACCTGAGCGATAGAGGCCTGGCCCTGAATGACTGTCACGCCGAGATCGTGTCCAGGCGCTCTCTGCTGCGCTTCCTGTACACCCAGCTGGAGCTGTATCTGAACAATGAGGATGACCAGAAGCGGAGCATCTTCCAGAAGTCCGAGCGCGGCGGCTTTCGGCTGAAGGAGAACATCCAGTTCCACCTGTACATCTCCACATCTCCATGCGGCGATGCCAGAATCTTTTCTCCACACGAGGCAATCCTGGAGGAGCCTGCAGACCGCCACCCAAACAGAAAGGCCAGGGGCCAGCTGCGGACCAAGATCGAGGCCGGCCAAGGCACAATCCCCGTGAGGAACAATGCCTCCATCCAGACCTGGGATGGCGTGCTGCAGGGAGAGCGCCTGCTGACAATGAGCTGTTCCGACAAGATCGCAAGGTGGAACGTGGTGGGCATCCAGGGCTCTCTGCTGAGCATCTTCGTGGAGCCCATCTACTTTTCCTCTATCATCCTGGGCTCTCTGTATCACGGCGATCACCTGAGCCGCGCCATGTACCAGCGGATCTCCAATATCGAGGACCTGCCACCCCTGTATACCCTGAACAAGCCTCTGCTGACAGGCATCTCTAATGCAGAGGCAAGGCAGCCAGGCAAGGCACCAATCTTCTCTGTGAATTGGACAGTGGGCGATAGCGCCATCGAAGTGATCAACGCCACCACAGGCAAGGGCGAGCTGGGAAGGGCCTCCCGGCTGTGCAAGCACGCCCTGTACTGTAGATGGATGAGGGTGCACGGCAAGGTGCCTTCTCACCTGCTGCGCAGCAAGATCACCAAGCCAAACGTGTACCACGAGACCAAGCTGGCCGCAAAGGAGTACCAGGCAGCAAAGGCAAGGCTGTTCACAGCCTTTATCAAGGCAGGGCTGGGGGCTTGGGTGGAAAAACCTACCGAGCAGGACCAGTTCTCACTGACACCTTAA

SEQ ID NO: 3 - protein, 4476 aa, Prevotella sp.
ATGAATATCCCCGCCCTGGTGGAAAACCAGAAGAAATACTTTGGAACTTACTCCGTGATGGCTATGCTGAATGCTCAGACCGTGCTGGATCACATCCAGAAGGTGGCCGACATCGAGGGCGAGCAGAACGAGAACAATGAGAATCTGTGGTTCCACCCCGTGATGAGCCACCTGTACAACGCCAAGAATGGCTATGATAAGCAGCCTGAAAAGACCATGTTTATCATCGAGCGCCTGCAGTCCTATTTCCCATTTCTGAAGATCATGGCCGAGAATCAGAGAGAGTACTCTAACGGCAAGTATAAGCAGAATAGGGTGGAGGTGAACAGCAATGACATCTTCGAGGTGCTGAAGCGCGCCTTTGGCGTGCTGAAGATGTACCGGGATCTGACCAACGCCTACAAGACCTACGAGGAGAAGCTGAATGACGGCTGCGAGTTCCTGACCTCCACAGAGCAGCCACTGTCTGGCATGATCAACAATTACTATACCGTGGCCCTGAGAAACATGAATGAGAGGTACGGCTACAAGACAGAGGATCTGGCCTTTATCCAGGACAAGCGGTTCAAGTTTGTGAAGGACGCCTACGGCAAGAAGAAGAGCCAGGTGAACACCGGCTTCTTTCTGTCTCTGCAGGATTACAATGGCGACACACAGAAGAAGCTGCACCTGAGCGGAGTGGGAATCGCCCTGCTGATCTGCCTGTTCCTGGATAAGCAGTATATCAACATCTTCCTGAGCCGCCTGCCCATCTTTAGCTCCTACAATGCCCAGTCCGAGGAGCGGAGAATCATCATCCGGAGCTTCGGCATCAACTCCATCAAGCTGCCAAAGGACAGAATCCACTCTGAGAAGAGCAACAAGTCCGTGGCTATGGACATGCTGAATGAGGTGAAGCGGTGTCCCGACGAGCTGTTTACCACACTGTCTGCCGAGAAGCAGAGCCGGTTCAGAATCATCTCCGACGATCACAACGAGGTGCTGATGAAGCGGAGCAGCGACCGGTTCGTGCCTCTGCTGCTGCAGTACATCGATTATGGCAAGCTGTTCGACCACATCAGATTTCACGTGAACATGGGCAAGCTGAGATACCTGCTGAAGGCCGATAAGACCTGCATCGACGGCCAGACAAGGGTGCGCGTGATCGAGCAGCCACTGAATGGCTTCGGCAGACTGGAGGAGGCCGAGACAATGAGGAAGCAGGAGAACGGCACATTTGGCAATAGCGGCATCCGGATCAGAGATTTCGAGAACATGAAGAGAGACGATGCCAACCCTGCCAATTACCCATATATCGTGGACACCTACACACACTATATCCTGGAGAACAATAAGGTGGAGATGTTCATCAATGATAAGGAGGACTCCGCCCCACTGCTGCCTGTGATCGAGGACGATCGCTATGTGGTGAAGACCATCCCCAGCTGTCGGATGTCCACACTGGAGATCCCTGCTATGGCCTTTCACATGTTCCTGTTTGGCAGCAAGAAGACCGAGAAGCTGATCGTGGACGTGCACAACCGGTACAAGAGACTGTTTCAGGCCATGCAGAAGGAGGAGGTGACAGCCGAGAATATCGCCAGCTTCGGCATCGCCGAGTCCGATCTGCCTCAGAAGATCCTGGACCTGATCTCCGGCAACGCCCACGGCAAGGATGTGGACGCCTTTATCAGACTGACCGTGGACGATATGCTGACCGATACAGAGAGGCGCATCAAGAGGTTCAAGGACGATAGAAAGTCTATCAGGAGCGCCGACAATAAGATGGGCAAGCGCGGCTTTAAGCAGATCAGCACCGGCAAGCTGGCCGATTTCCTGGCCAAGGACATCGTGCTGTTTCAGCCCAGCGTGAACGATGGCGAGAATAAGATCACAGGCCTGAACTACCGGATCATGCAGTCCGCCATCGCCGTGTACGACTCTGGCGACGATTATGAGGCCAAGCAGCAGTTCAAGCTGATGTTTGAGAAGGCCAGACTGATCGGCAAGGGCACCACAGAGCCTCACCCATTCCTGTATAAGGTGTTTGCCAGGTCCATCCCTGCCAACGCCGTGGAGTTTTACGAGAGATACCTGATCGAGCGGAAGTTCTACCTGACCGGCCTGTCTAACGAGATCAAGAAGGGCAATAGAGTGGATGTGCCCTTCATCCGGCGGGACCAGAATAAGTGGAAGACCCCTGCCATGAAGACACTGGGCAGAATCTACAGCGAGGATCTGCCCGTGGAGCTGCCTAGGCAGATGTTCGACAACGAGATCAAGTCTCACCTGAAGAGCCTGCCCCAGATGGAGGGCATCGACTTTAACAATGCCAATGTGACCTACCTGATCGCCGAGTATATGAAGAGGGTGCTGGACGATGACTTCCAGACCTTCTACCAGTGGAACAGGAATTACCGCTATATGGATATGCTGAAGGGCGAGTACGACCGCAAGGGCAGCCTGCAGCACTGCTTCACCTCCGTGGAGGAGAGGGAGGGCCTGTGGAAGGAGAGGGCCTCCAGAACAGAGCGGTACAGAAAGCAGGCCTCCAACAAGATCAGGTCTAATCGCCAGATGCGGAACGCCTCCTCTGAGGAGATCGAGACAATCCTGGATAAGAGACTGAGCAACTCCAGGAATGAGTACCAGAAGAGCGAGAAAGTGATCAGGCGCTATAGAGTGCAGGACGCCCTGCTGTTCCTGCTGGCCAAGAAGACCCTGACAGAGCTGGCCGATTTCGACGGCGAGAGGTTTAAGCTGAAGGAGATCATGCCAGACGCCGAGAAGGGCATCCTGTCTGAGATCATGCCCATGAGCTTCACCTTTGAGAAGGGCGGCAAGAAGTATACCATCACAAGCGAGGGCATGAAGCTGAAGAACTACGGCGATTTCTTTGTGCTGGCCTCCGACAAGAGAATCGGCAATCTGCTGGAGCTGGTGGGCTCCGACATCGTGAGCAAGGAGGACATCATGGAGGAGTTCAACAAGTATGATCAGTGTAGGCCCGAGATCAGCTCCATCGTGTTCAATCTGGAGAAGTGGGCCTTTGACACCTACCCTGAGCTGTCCGCCAGAGTGGATAGGGAGGAGAAGGTGGACTTTAAGTCTATCCTGAAGATCCTGCTGAACAATAAGAACATCAATAAGGAGCAGTCCGACATCCTGCGCAAGATCCGGAACGCCTTCGATGCCAACAATTATCCAGACAAGGGCGTGGTGGAGATCAAGGCCCTGCCCGAGATCGCCATGTCTATCAAGAAGGCCTTTGGCGAGTACGCCATCATGAAGGGCAGCCTGCAGCTGCCACCTCTGGAGCGCCTGACCCTGGGATCCCAGCTGCACCTGCCTCAGGTGCTGGCCGACGCCGTGTCTCGGCTGGTGATCGGCAAGTTCGGCGATCTGACCGACAACTTTTCTAGCCCACACGCAAGGAGAATCGGCCTGGCCGGAGTGGTCATGACCACAGGCACAGATGTGAAGGACGCCAAAGTGATCTGCGTGAGCACCGGCAGCAAGTGCATCAACGGCGAGTACCTGAGCGATAGAGGCCTGGCCCTGAATGACTGTCACGCCGAGATCGTGTCCAGGCGCTCTCTGCTGCGCTTCCTGTACACCCAGCTGGAGCTGTATCTGAACAATGAGGATGACCAGAAGCGGAGCATCTTCCAGAAGTCCGAGCGCGGCGGCTTTCGGCTGAAGGAGAACATCCAGTTCCACCTGTACATCTCCACATCTCCATGCGGCGATGCCAGAATCTTTTCTCCACACGAGGCAATCCTGGAGGAGCCTGCAGACCGCCACCCAAACAGAAAGGCCAGGGGCCAGCTGCGGACCAAGATCGAGGCCGGCCAAGGCACAATCCCCGTGAGGAACAATGCCTCCATCCAGACCTGGGATGGCGTGCTGCAGGGAGAGCGCCTGCTGACAATGAGCTGTTCCGACAAGATCGCAAGGTGGAACGTGGTGGGCATCCAGGGCTCTCTGCTGAGCATCTTCGTGGAGCCCATCTACTTTTCCTCTATCATCCTGGGCTCTCTGTATCACGGCGATCACCTGAGCCGCGCCATGTACCAGCGGATCTCCAATATCGAGGACCTGCCACCCCTGTATACCCTGAACAAGCCTCTGCTGACAGGCATCTCTAATGCAGAGGCAAGGCAGCCAGGCAAGGCACCAATCTTCTCTGTGAATTGGACAGTGGGCGATAGCGCCATCGAAGTGATCAACGCCACCACAGGCAAGGGCGAGCTGGGAAGGGCCTCCCGGCTGTGCAAGCACGCCCTGTACTGTAGATGGATGAGGGTGCACGGCAAGGTGCCTTCTCACCTGCTGCGCAGCAAGATCACCAAGCCAAACGTGTACCACGAGACCAAGCTGGCCGCAAAGGAGTACCAGGCAGCAAAGGCAAGGCTGTTCACAGCCTTTATCAAGGCAGGGCTGGGGGCTTGGGTGGAAAAACCTACCGAGCAGGACCAGTTCTCACTGACACCTTAA

SEQ ID NO: 4 - skipped

SEQ ID NO: 5 - RNA, 36 nt, Prevotella sp.
  source 1..36: mol_type = other RNA
gttgtggaaggtccagttttgaggggctattacaac

SEQ ID NO: 6 - RNA, 36 nt, Riemerella anatipestifer
  source 1..36: mol_type = other RNA
gttgggactgctctcactttgaagggtattcacaac

SEQ ID NO: 7 - RNA, 30 nt, Prevotella sp.
  source 1..30: mol_type = other RNA
agagtaatgagctcgaacatgctgtggagt

SEQ ID NO: 8 - RNA, 30 nt, Prevotella sp.
  source 1..30: mol_type = other RNA
agaagctggagagtaatgagctcgaacatg

SEQ ID NO: 9 - RNA, 30 nt, Prevotella sp.
  source 1..30: mol_type = other RNA
aaccccaggccaccgtcagtgctctgctcc

SEQ ID NO: 10 - RNA, 30 nt, Prevotella sp.
  source 1..30: mol_type = other RNA
  modified_base 1..3: mod_base = OTHER; note = methylated 1..3
ggccaccgtcagtgccctgctccctcgaat

SEQ ID NO: 11 - RNA, 30 nt, Prevotella sp.
  source 1..30: mol_type = other RNA
  modified_base 1..3: mod_base = OTHER; note = methylated 1..3
ccaccgtcagtgccctgctccctcgaatga

SEQ ID NO: 12 - RNA, 40 nt, Prevotella sp.
  source 1..40: mol_type = other RNA
taaccccaggccaccgtcagtgccctgctccctcgaatga

SEQ ID NO: 13 - RNA, 30 nt, Prevotella sp.
  source 1..30: mol_type = other RNA
ggtcacgtcgtagtactgctggcggaggtg

SEQ ID NO: 14 - RNA, 30 nt, Prevotella sp.
  source 1..30: mol_type = other RNA
  modified_base 1..3: mod_base = OTHER; note = methylated 1..3
tcgtagtactgctggcggaggtgcgcgtgc

SEQ ID NO: 15 - RNA, 30 nt, Prevotella sp.
  source 1..30: mol_type = other RNA
gtagtactgctggcggaggtgcgcgtgctg

SEQ ID NO: 16 - RNA, 30 nt, Prevotella sp.
  source 1..30: mol_type = other RNA
gacgagtcggtgccggactggcgcgagtgc

SEQ ID NO: 17 - RNA, 30 nt, Prevotella sp.
  source 1..30: mol_type = other RNA
cggtgccggactggcgcgagtgcgagcccg

SEQ ID NO: 18 - RNA, 30 nt, Prevotella sp.
  source 1..30: mol_type = other RNA
gtgccggactggcgcgagtgcgagcccgaa

SEQ ID NO: 19 - RNA, 30 nt, Prevotella sp.
  source 1..30: mol_type = other RNA
ggacgcgggcgactagtgcgagcggacatg

SEQ ID NO: 20 - RNA, 30 nt, Prevotella sp.
  source 1..30: mol_type = other RNA
ggcgactagtgcgagcggacatgctgggca

SEQ ID NO: 21 - RNA, 30 nt, Prevotella sp.
  source 1..30: mol_type = other RNA
cgactagtgcgagcggacatgctgggcacc

SEQ ID NO: 22 - RNA, 30 nt, Prevotella sp.
  source 1..30: mol_type = other RNA
caaaataagactgtcgaagatgctgagctg

SEQ ID NO: 23 - RNA, 30 nt, Prevotella sp.
  source 1..30: mol_type = other RNA
caaaacaagactgtcgaagatgctgagctg

SEQ ID NO: 24 - RNA, 30 nt, Prevotella sp.
  source 1..30: mol_type = other RNA
cctgcatcagtaccggcctgtcgggagtgg

SEQ ID NO: 25 - RNA, 30 nt, Prevotella sp.
  source 1..30: mol_type = other RNA
agtaccggcctgtcgggagtgggatttggg

SEQ ID NO: 26 - RNA, 30 nt, Prevotella sp.
  source 1..30: mol_type = other RNA
aaaggatctgagccattggtcgtcattgtt

SEQ ID NO: 27 - RNA, 30 nt, Prevotella sp.
  source 1..30: mol_type = other RNA
tgagccattggtcgtcattgttctcaattc

SEQ ID NO: 28 - RNA, 30 nt, Prevotella sp.
  source 1..30: mol_type = other RNA
gtaccctcatctcgagagtgataggtgcca

SEQ ID NO: 29 - RNA, 36 nt, Prevotella sp.
  source 1..36: mol_type = other RNA
ctgtctgtaccctcatctcgagagtgataggtgcca

SEQ ID NO: 30 - RNA, 30 nt, Prevotella sp.
  source 1..30: mol_type = other RNA
atgcttagtccaccgtctgtactctcatct

SEQ ID NO: 31 - RNA, 36 nt, Prevotella sp.
  source 1..36: mol_type = other RNA
cttagtccaccgtctgtactctcatctcgagagtga

SEQ ID NO: 32 - RNA, 30 nt, Prevotella sp.
  source 1..30: mol_type = other RNA
gcttagtccactgcctgtactctcatctcg

SEQ ID NO: 33 - RNA, 36 nt, Prevotella sp.
  source 1..36: mol_type = other RNA
tagtccactgcctgtactctcatctcgagagtgata

SEQ ID NO: 34 - RNA, 75 nt, Prevotella sp.
  source 1..75: mol_type = other RNA
agagtaatgagctcgaacatgctgtggagtgttgtggaaggtccagttttgaggggctattacaacttmcmgmat

SEQ ID NO: 35 - RNA, 75 nt, Prevotella sp.
  source 1..75: mol_type = other RNA
agaagctggagagtaatgagctcgaacatggttgtggaaggtccagttttgaggggctattacaacttmcmgmat

SEQ ID NO: 36 - RNA, 75 nt, Prevotella sp.
  source 1..75: mol_type = other RNA
aaccccaggccaccgtcagtgctctgctccgttgtggaaggtccagttttgaggggctattacaacttmcmgmat

SEQ ID NO: 37 - RNA, 72 nt, Prevotella sp.
  source 1..72: mol_type = other RNA
ggccaccgtcagtgccctgctccctcgaatgttgtggaaggtccagttttgaggggctattacaacttcgat

SEQ ID NO: 38 - RNA, 75 nt, Prevotella sp.
  source 1..75: mol_type = other RNA
ccaccgtcagtgccctgctccctcgaatgagttgtggaaggtccagttttgaggggctattacaacttmcmgmat

SEQ ID NO: 39 - RNA, 82 nt, Prevotella sp.
  source 1..82: mol_type = other RNA
taaccccaggccaccgtcagtgccctgctccctcgaatgagttgtggaaggtccagttttgaggggctattacaacttcgat

SEQ ID NO: 40 - RNA, 72 nt, Prevotella sp.
  source 1..72: mol_type = other RNA
ggtcacgtcgtagtactgctggcggaggtggttgtggaaggtccagttttgaggggctattacaacttcgat

SEQ ID NO: 41 - RNA, 72 nt, Prevotella sp.
  source 1..72: mol_type = other RNA
tcgtagtactgctggcggaggtgcgcgtgcgttgtggaaggtccagttttgaggggctattacaacttcgat

SEQ ID NO: 42 - RNA, 72 nt, Prevotella sp.
  source 1..72: mol_type = other RNA
gtagtactgctggcggaggtgcgcgtgctggttgtggaaggtccagttttgaggggctattacaacttcgat

SEQ ID NO: 43 - RNA, 72 nt, Prevotella sp.
  source 1..72: mol_type = other RNA
gacgagtcggtgccggactggcgcgagtgcgttgtggaaggtccagttttgaggggctattacaacttcgat

SEQ ID NO: 44 - RNA, 72 nt, Prevotella sp.
  source 1..72: mol_type = other RNA
cggtgccggactggcgcgagtgcgagcccggttgtggaaggtccagttttgaggggctattacaacttcgat

SEQ ID NO: 45 - RNA, 72 nt, Prevotella sp.
  source 1..72: mol_type = other RNA
gtgccggactggcgcgagtgcgagcccgaagttgtggaaggtccagttttgaggggctattacaacttcgat

SEQ ID NO: 46 - RNA, 72 nt, Prevotella sp.
  source 1..72: mol_type = other RNA
ggacgcgggcgactagtgcgagcggacatggttgtggaaggtccagttttgaggggctattacaacttcgat

SEQ ID NO: 47 - RNA, 72 nt, Prevotella sp.
  source 1..72: mol_type = other RNA
ggcgactagtgcgagcggacatgctgggcagttgtggaaggtccagttttgaggggctattacaacttcgat

SEQ ID NO: 48 - RNA, 72 nt, Prevotella sp.
  source 1..72: mol_type = other RNA
cgactagtgcgagcggacatgctgggcaccgttgtggaaggtccagttttgaggggctattacaacttcgat

SEQ ID NO: 49 - RNA, 72 nt, Prevotella sp.
  source 1..72: mol_type = other RNA
caaaataagactgtcgaagatgctgagctggttgtggaaggtccagttttgaggggctattacaacttcgat

SEQ ID NO: 50 - RNA, 72 nt, Prevotella sp.
  source 1..72: mol_type = other RNA
caaaacaagactgtcgaagatgctgagctggttgtggaaggtccagttttgaggggctattacaacttcgat

SEQ ID NO: 51 - RNA, 72 nt, Prevotella sp.
  source 1..72: mol_type = other RNA
cctgcatcagtaccggcctgtcgggagtgggttgtggaaggtccagttttgaggggctattacaacttcgat

SEQ ID NO: 52 - RNA, 72 nt, Prevotella sp.
  source 1..72: mol_type = other RNA
agtaccggcctgtcgggagtgggatttggggttgtggaaggtccagttttgaggggctattacaacttcgat

SEQ ID NO: 53 - RNA, 72 nt, Prevotella sp.
  source 1..72: mol_type = other RNA
aaaggatctgagccattggtcgtcattgttgttgtggaaggtccagttttgaggggctattacaacttcgat

SEQ ID NO: 54 - RNA, 72 nt, Prevotella sp.
  source 1..72: mol_type = other RNA
tgagccattggtcgtcattgttctcaattcgttgtggaaggtccagttttgaggggctattacaacttcgat

SEQ ID NO: 55 - RNA, 72 nt, Prevotella sp.
  source 1..72: mol_type = other RNA
gtaccctcatctcgagagtgataggtgccagttgtggaaggtccagttttgaggggctattacaacttcgat

SEQ ID NO: 56 - RNA, 78 nt, Prevotella sp.
  source 1..78: mol_type = other RNA
ctgtctgtaccctcatctcgagagtgataggtgccagttgtggaaggtccagttttgaggggctattacaacttcgat

SEQ ID NO: 57 - RNA, 72 nt, Prevotella sp.
  source 1..72: mol_type = other RNA
atgcttagtccaccgtctgtactctcatctgttgtggaaggtccagttttgaggggctattacaacttcgat

SEQ ID NO: 58 - RNA, 78 nt, Prevotella sp.
  source 1..78: mol_type = other RNA
cttagtccaccgtctgtactctcatctcgagagtgagttgtggaaggtccagttttgaggggctattacaacttcgat

SEQ ID NO: 59 - RNA, 72 nt, Prevotella sp.
  source 1..72: mol_type = other RNA
gcttagtccactgcctgtactctcatctcggttgtggaaggtccagttttgaggggctattacaacttcgat

SEQ ID NO: 60 - RNA, 78 nt, Prevotella sp.
  source 1..78: mol_type = other RNA
tagtccactgcctgtactctcatctcgagagtgatagttgtggaaggtccagttttgaggggctattacaacttcgat

SEQ ID NO: 61 - DNA, 1203 bp, synthetic construct
  source 1..1203: mol_type = other DNA
atgaatccggcctcggcgccccctccgctcccgccgcctgggcagcaagtgatccacgtcacgcaggacctagacacagacctcgaagccctcttcaactctgtcatgaatccgaagcctagctcgtggcggaagaagatcctgccggagtctttctttaaggagcctgattcgggctcgcactcgcgccagtccagcaccgactcgtcgggcggccacccggggcctcgactggctgggggtgcccagcatgtccgctcgcactcgtcgcccgcgtccctgcagctgggcaccggcgcgggtgctgcgggtagccccgcgcagcagcacgcgcacctccgccagcagtcctacgacgtgaccgacgagctgccactgcccccgggctgggagatgaccttcacggccactggccagaggtacttcctcaatcacatagaaaaaatcaccacatggcaagaccctaggaaggcgatgaatcagcctctgaatcatatgaacctccaccctgccgtcagttccacaccagtgcctcagaggtccatggcagtatcccagccaaatctcgtgatgaatcaccaacaccagcagcagatggcccccagtaccctgagccagcagaaccaccccactcagaacccacccgcagggctcatgagtatgcccaatgcgctgaccactcagcagcagcagcagcagaaactgcggcttcagagaatccagatggagagagaaaggattcgaatgcgccaagaggagctcatgaggcaggaagctgccctctgtcgacagctccccatggaagctgagactcttgccccagttcaggctgctgtcaacccacccacgatgaccccagacatgagatccatcactaataatagctcagatcctttcctcaatggagggccatatcattcgagggagcagagcactgacagtggcctggggttagggtgctacagtgtccccacaactccggaggacttcctcagcaatgtggatgagatggatacaggagaaaacgcaggacaaacacccatgaacatcaatccccaacagacccgtttccctgatttccttgactgtcttccaggaacaaacgttgacttaggaactttggaatctgaagacctgatccccctcttcaatgatgtagagtctgctctgaacaaaagtgagccctttctaacctggctgtaa

SEQ ID NO: 62 - DNA, 1467 bp, synthetic construct
  source 1..1467: mol_type = other DNA
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatag

SEQ ID NO: 63 - DNA, 1515 bp, synthetic construct
  source 1..1515: mol_type = other DNA
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggcaatgcggaatatcaatcccagcacagcaaattctccaaaatgtcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatag

SEQ ID NO: 64 - protein, 1541 aa, Prevotella sp.
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTSEQIDNOREGULARDPSPCASBBOLDGSNESGSLINKERITALICS

SEQ ID NO: 65 - protein, 1495 aa, Prevotella sp.
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLGSGGGGSQLHLPQVLADAVSRLVLGKFGDLTDNFSSPHARRKVLAGVVMTTGTDVKDAKVISVSTGGKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 66 - protein, 1494 aa, Riemerella anatipestifer
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLANYYSAYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVANAFCANQYPFYKEALHAPIPLFTVAQPTTEEKDGLGIAEALLKVLREYCEIVKSQIGSSLQKKLEELELQLHLPQVLADAVSRLVIGKFGDLTDNFSSPHARRIGLAGVVMTTGTDVKDAKVICVSTGSKCINGEYLSDRGLALNDCHAEIVSRRSLLRFLYTQLELYLNNEDDQKRSIFQKSERGGFRLKENIQFHLYISTSPCGDARIFSPHEAILEEPADRHPNRKARGQLRTKIEAGQGTIPVRNNASIQTWDGVLQGERLLTMSCSDKIARWNVVGIQGSLLSIFVEPIYFSSIILGSLYHGDHLSRAMYQRISNIEDLPPLYTLNKPLLTGISNAEARQPGKAPIFSVNWTVGDSAIEVINATTGKGELGRASRLCKHALYCRWMRVHGKVPSHLLRSKITKPNVYHETKLAAKEYQAAKARLFTAFIKAGLGAWVEKPTEQDQFSLTP

SEQ ID NO: 67 - protein, 1489 aa, Prevotella sp.
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLQLHLPQVLADAVSRLVIGKFGDLTDNFSSPHARRIGLAGVVMTTGTDVKDAKVICVSTGSKCINGEYLSDRGLALNDCHAEIVSRRSLLRFLYTQLELYLNNEDDQKRSIFQKSERGGFRLKENIQFHLYISTSPCGDARIFSPHEAILEEPADRHPNRKARGQLRTKIEAGQGTIPVRNNASIQTWDGVLQGERLLTMSCSDKIARWNVVGIQGSLLSIFVEPIYFSSIILGSLYHGDHLSRAMYQRISNIEDLPPLYTLNKPLLTGISNAEARQPGKAPIFSVNWTVGDSAIEVINATTGKGELGRASRLCKHALYCRWMRVHGKVPSHLLRSKITKPNVYHETKLAAKEYQAAKARLFTAFIKAGLGAWVEKPTEQDQFSLTP

SEQ ID NO: 68 - protein, 7 aa, synthetic construct
GSGGGGS

SEQ ID NO: 69 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 70 - protein, 145 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKAPV

SEQ ID NO: 71 - protein, 198 aa, Homo sapiens [sequence omitted]

SEQ ID NO: 72 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 73 - protein, 198 aa, Canis sp.
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 74 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 75 - protein, 239 aa, Rattus sp.
MAVGSKPKAALVGPHWERERIWCFLCSTGLGTQQTGQTSRWLRPAATQDPVSPPRSLLMKQRKFLYHFKNVRWAKGRHETYLCYVVKRRDSATSFSLDFGYLRNKSGCHVELLFLRYISDWDLDPGRCYRVTWFTSWSPCYDCARHVADFLRGNPNLSLRIFTARLTGWGALPAGLMSPARPSDYFYCWNTFVENHERTFKAWEGLHENSVRLSRRLRRILLPLYEVDDLRDAFRTLGL

SEQ ID NO: 76 - protein, 198 aa, Canis lupus; identical to SEQ ID NO: 73
  source 1..198: note = "subspecies" familiaris

SEQ ID NO: 77 - protein, 199 aa, Bos taurus; identical to SEQ ID NO: 74

SEQ ID NO: 78 - protein, 198 aa, Mus musculus [sequence omitted]

SEQ ID NO: 79 - protein, 229 aa, Rattus norvegicus [sequence omitted]

SEQ ID NO: 80 - protein, 229 aa, Mesocricetus auratus
MSSETGPVVVDPTLRRRIEPHEFDAFFDQGELRKETCLLYEIRWGGRHNIWRHTGQNTSRHVEINFIEKFTSERYFYPSTRCSIVWFLSWSPCGECSKAITEFLSGHPNVTLFIYAARLYHHTDQRNRQGLRDLISRGVTIRIMTEQEYCYCWRNFVNYPPSNEVYWPRYPNLWMRLYALELYCIHLGLPPCLKIKRRHQYPLTFFRLNLQSCHYQRIPPHILWATGFI

SEQ ID NO: 81 - protein, 236 aa, Pongo pygmaeus
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 82 - protein, 236 aa, Oryctolagus cuniculus
MASEKGPSNKDYTLRRRIEPWEFEVFFDPQELRKEACLLYEIKWGASSKTWRSSGKNTTNHVEVNFLEKLTSEGRLGPSTCCSITWFLSWSPCWECSMAIREFLSQHPGVTLIIFVARLFQHMDRRNRQGLKDLVTSGVTVRVMSVSEYCYCWENFVNYPPGKAAQWPRYPPRWMLMYALELYCIILGLPPCLKISRRHQKQLTFFSLTPQYCHYKMIPPYILLATGLLQPSVPWR

SEQ ID NO: 83 - protein, 235 aa, Monodelphis domestica
MNSKTGPSVGDATLRRRIKPWEFVAFFNPQELRKETCLLYEIKWGNQNIWRHSNQNTSQHAEINFMEKFTAERHFNSSVRCSITWFLSWSPCWECSKAIRKFLDHYPNVTLAIFISRLYWHMDQQHRQGLKELVHSGVTIQIMSYSEYHYCWRNFVDYPQGEEDYWPKYPYLWIMLYVLELHCIILGLPPCLKISGSHSNQLALFSLDLQDCHYQKIPYNVLVATGLVQPFVTWR

SEQ ID NO: 84 - protein, 224 aa, Pongo pygmaeus
MAQKEEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEELEIQDALKKLKEAGCKLRIMKPQDFEYVWQNFVEQEEGESKAFQPWEDIQENFLYYEEKLADILK

SEQ ID NO: 85 - protein, 224 aa, Bos taurus
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 86 - protein, 440 aa, Mus musculus
MQPQRLGPRAGMGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSSSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 87 - protein, 429 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 88 - protein, 430 aa, Rattus sp.
MGPFCLGCSHRKCYSPIRNLISQETFKFHFKNRLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 89 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 90 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 91 - protein, 370 aa, Rhesus macaque
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 92 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 93 - protein, 377 aa, Chlorocebus sabaeus
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 94 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 95 - protein, 373 aa, Homo sapiens; identical to SEQ ID NO: 90

SEQ ID NO: 96 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 97 - protein, 395 aa, Rattus sp.
MQPQGLGPNAGMGPVCLGCSHRRPYSPIRNPLKKLYQQTFYFHFKNVRYAWGRKNNFLCYEVNGMDCALPVPLRQGVFRKQGHIHAELCFIYWFHDKVLRVLSPMEEFKVTWYMSWSPCSKCAEQVARFLAAHRNLSLAIFSSRLYYYLRNPNYQQKLCRLIQEGVHVAAMDLPEFKKCWNKFVDNDGQPFRPWMRLRINFSFYDCKLQEIFSRMNLLREDVFYLQFNNSHRVKPVQNRYYRRKSYLCYQLERANGQEPLKGYLLYKKGEQHVEILFLEKMRSMELSQVRITCYLTWSPCPNCARQLAAFKKDHPDLILRIYTSRLYFWRKKFQKGLCTLWRSGIHVDVMDLPQFADCWTNFVNPQRPFRPWNELEKNSWRIQRRLRRIKESWGL

SEQ ID NO: 98 - protein, 227 aa, Bos sp.
MDGWEVAFRSGTVLKAGVLGVSMTEGWAGSGHPGQGACVWTPGTRNTMNLLREVLFKQQFGNQPRVPAPYYRRKTYLCYQLKQRNDLTLDRGCFRNKKQRHAERFIDKINSLDLNPSQSYKIICYITWSPCPNCANELVNFITRNNHLKLEIFASRLYFHWIKSFKMGLQDLQNAGISVAVMTHTEFEDCWEQFVDNQSRPFQPWDKLEQYSASIRRRLQRILTAPI

SEQ ID NO: 99 - protein, 490 aa, Pan troglodytes
MNPQIRNPMEWMYQRTFYYNFENEPILYGRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNEGQPFMPWYKFDDNYAFLHRTLKEIIRHLMDPDTFTFNFNNDPLVLRRHQTYLCYEVERLDNGTWVLMDQHMGFLCNEAKNLLCGFYGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRQGCPFQPWDGLEEHSQALSGRLRAILQVRASSLCMVPHRPPPPPQSPGPCLPLCSEPPLGSLLPTGRPAPSLPFLLTASFSFPPPASLPPLPSLSLSPGHLPVPSFHSLTSCSIQPPCSSRIRETEGWASVSKEGRDLG

SEQ ID NO: 100 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 101 - protein, 190 aa, Gorilla sp.
MNPQIRNPMKAMYPGTFYFQFKNLWEANDRNETWLCFTVEGIKRRSVVSWKTGVFRNQVDSETHCHAERCFLSWECDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 102 - protein, 199 aa, Homo sapiens; identical to SEQ ID NO: 89

SEQ ID NO: 103 - protein, 202 aa, Rhesus macaque
MDGSPASRPRHLMDPNTFTFNFNNDLSVRGRHQTYLCYEVERLDNGTWVPMDERRGFLCNKAKNVPCGDYGCHVELRFLCEVPSWQLDPAQTYRVTWFISWSPCFRRGCAGQVRVFLQENKHVRLRIFAARIYDYDPLYQEALRTLRDAGAQVSIMTYEEFKHCWDTFVDRQGRPFQPWDGLDEHSQALSGRLRAILQNQGN

SEQ ID NO: 104 - protein, 185 aa, Bos sp.
MDEYTFTENFNNQGWPSKTYLCYEMERLDGDATIPLDEYKGFVRNKGLDQPEKPCHAELYFLGKIHSWNLDRNQHYRLTCFISWSPCYDCAQKLTTFLKENHHISLHILASRIYTHNRFGCHQSGLCELQAAGARITIMTFEDFKHCWETFVDHKGKPFQPWEGLNVKSQALCTELQAILKTQQN

SEQ ID NO: 105 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 106 - protein, 210 aa, Rhesus macaque
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 107 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 108 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 109 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 110 - protein, 229 aa, Rattus sp. [sequence omitted]

SEQ ID NO: 111 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 112 - protein, 224 aa, Mus sp.
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 113 - protein, 224 aa, Rattus sp.
MAQKEEAAEAAAPASQNGDDLENLEDPEKLKELIDLPPFEIVTGVRLPVNFFKFQFRNVEYSSGRNKTFLCYVVEAQSKGGQVQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYLWQNFVEQEEGESKAFEPWEDIQENFLYYEEKLADILK

SEQ ID NO: 114 - protein, 224 aa, Bos sp.; identical to SEQ ID NO: 85

SEQ ID NO: 115 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSFMIQVKILHTTKSPAV

SEQ ID NO: 116 - protein, 381 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKFNYDEFQHCWSKFVYSQRELFEPWNNLPKYYILLHFMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKKHVSLCIFTARIYRRQGRCQEGLRTLAEAGAKISFTYSEFKHCWDTFVDHQGCPFQPWDGLDEHSQDLSGRLRAILQNQEN

SEQ ID NO: 117 - protein, 182 aa, Homo sapiens
MDPPTFTFNFNNEPWWGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISFTYSEFKHCWDTFVDHQGCPFQPWDGLDEHSQDLSGRLRAILQ

SEQ ID NO: 118 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISFMTYSEFKHCWDTFVDHQGCPFQPWDGLDEHSQDLSGRLRAILQ

SEQ ID NO: 119 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 120 - protein, 198 aa, Mus musculus; identical to SEQ ID NO: 72

SEQ ID NO: 121 - protein, 388 aa, Rattus norvegicus
MEPLYEEYLTHSGTIVKPYYWLSVSLNCTNCPYHIRTGEEARVPYTEFHQTFGFPWSTYPQTKHLTFYELRSSSGNLIQKGLASNCTGSHTHPESMLFERDGYLDSLIFHDSNIRHIILYSNNSPCDEANHCCISKMYNFLMNYPEVTLSVFFSQLYHTENQFPTSAWNREALRGLASLWPQVTLSAISGGIWQSILETFVSGISEGLTAVRPFTAGRTLTDRYNAYEINCITEVKPYFTDALHSWQKENQDQKVWAASENQPLHNTTPAQWQPDMSQDCRTPAVFMLVPYRDLPPIHVNPSPQKPRTVVRHLNTLQLSASKVKALRKSPSGRPVKKEEARKGSTRSQEANETNKSKWKKQTLFIKSNICHLLEREQKKIGILSSWSV

SEQ ID NO: 122 - protein, 363 aa, Macaca fascicularis
MEPTYEEYLANHGTIVKPYYWLSFSLDCSNCPYHIRTGEEARVSLTEFCQIFGFPYGTTYPQTKHLTFYELKTSSGSLVQKGHASSCTGNYIHPESMLFEMNGYLDSAIYNNDSIRHIILYCNNSPCNEANHCCISKVYNFLITYPGITLSIYFSQLYHTEMDFPASAWNREALRSLASLWPRVVLSPISGGIWHSVLHSFVSGVSGSHVFQPILTGRALTDRYNAYEINAITGVKPFFTDVLLHTKRNPNTKAQMALESYPLNNAFPGQSFQMTSGIPPDLRAPVVFVLLPLRDLPPMHMGQDPNKPRNIIRHLNMPQMSFQETKDLERLPTRRSVETVEITERFASSKQAEEKTKKKKGKK

SEQ ID NO: 123 - protein, 224 aa, Petrozon marinus
MAGYECVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLTMHFSRIYDRDREGDHRGLRGLKHVSNSFRMGVVGRAEVKECLAEYVEASRRTLTWLDTTESMAAKMRRKLFCILVRCAGMRESGIPLHLFTLQTPLLSGRVVWWRV

SEQ ID NO: 124 - protein, 331 aa, Petrozon marinus
MELREVVDCALASCVRHEPLSRVAFLRCFAAPSQKPRGTVILFYVEGAGRGVTGGHAVNYNKQGTSIHAEVLLLSAVRAALLRRRRCEDGEEATRGCTLHCYSTYSPCRDCVEYIQEFGASTGVRVVIHCCRLYELDVNRRRSEAEGVLRSLSRLGRDFRLMGPRDAIALLLGGRLANTADGESGASGNAWVTETNVVEPLVDMTGFGDEDLHAQVQRNKQIREAYANYASAVSLMLGELHVDPDKFPFLAEFLAQTSVEPSGTPRETRGRPRGASSRGPEIGRQRPADFERALGAYGLFLHPRIVSREADREEIKRDLIVVMRKHNYQGP

SEQ ID NO: 125 - protein, 209 aa, Petrozon marinus
MAGDENVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLMMHFSRIYDRDREGDHRGLRGLKHVSNSFRMGVVGRAEVKECLAEYVEASRRTLTWLDTTESMAAKMRRKLFCILVRCAGMRESGMPLHLFT

SEQ ID NO: 126 - protein, 158 aa, Saccharomyces cerevisiae
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 127 - protein, 218 aa, Rattus sp.
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRGLPPCLNILRRKQPQLTFFTIALQSCHYQRLPPHILWATGLK

SEQ ID NO: 128 - protein, 218 aa, Rattus sp.
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQHYQRLPPHILWATGLK

SEQ ID NO: 129 - protein, 429 aa, Mus sp.; identical to SEQ ID NO: 87

SEQ ID NO: 130 - protein, 167 aa, unidentified
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 131 - protein, 167 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 132 - protein, 178 aa, synthetic construct
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 133 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 134 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 135 - protein, 183 aa, Salmonella typhimurium
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 136 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 137 - protein, 171 aa, Haemophilus influenza
MDAAKVRSEFDEKMMRYALELADKAEALGEIPVGAVLVDDARNIIGEGWNLSIVQSDPTAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 138 - protein, 161 aa, Caulobacter crescentus
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 139 - protein, 179 aa, Geobacter sulfurreducens
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 140 - protein, 167 aa, Escherichia coli; identical to SEQ ID NO: 130

SEQ ID NO: 141 - protein, 167 aa, synthetic construct; identical to SEQ ID NO: 130

SEQ ID NO: 142 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCTFFRMPRQVFNAQKKAQSSTD